SEQUENCE LISTING (4 sequences)

SEQ ID NO: 1 - DNA, 897 bp, Homo sapiens
atgacctcgctgacccagcgcagctccggcctggtgcagcggcgcaccgaggcctcccgcaacgccgccgacaaggagcgggcggcgggcggcggcgccggcagcagcgaggacgacgcgcagagccgccgcgacgagcaggacgacgacgacaagggcgactccaaggaaacgcggctgaccctgatggaggaagtgctcctgctgggcctcaaggaccgcgagggttacacatcattttggaatgactgtatatcatctggattacgtggctgtatgttaattgaattagcattgagaggaaggttacaactagaggcttgtggaatgagacgtaaaagtctattaacaagaaaggtaatctgtaagtcagatgctccaacaggggatgttcttcttgatgaagctctgaagcatgttaaggaaactcagcctccagaaacggtccagaactggattgaattacttagtggtgagacatggaatccattaaaattgcattatcagttaagaaatgtacgggaacgattagctaaaaacctggtggaaaagggtgtattgacaacagagaaacagaacttcctactttttgacatgacaacacatcccctcaccaataacaacattaagcagcgcctcatcaagaaagtacaggaagccgttcttgacaaatgggtgaatgaccctcaccgcatggacaggcgcttgctggccctcatttacctggctcatgcctcggacgtcctggagaatgcttttgctcctcttctggacgagcagtatgatttggctaccaagagagtgcggcagcttctcgacttagaccctgaagtggaatgtctgaaggccaacaccaatgaggttctgtgggcggtggtggcggcgttcaccaagtaa

SEQ ID NO: 2 - protein, 298 aa, Homo sapiens
MTSLTQRSSGLVQRRTEASRNAADKERAAGGGAGSSEDDAQSRRDEQDDDDKGDSKETRLTLMEEVLLLGLKDREGYTSFWNDCISSGLRGCMLIELALRGRLQLEACGMRRKSLLTRKVICKSDAPTGDVLLDEALKHVKETQPPETVQNWIELLSGETWNPLKLHYQLRNVRERLAKNLVEKGVLTTEKQNFLLFDMTTHPLTNNNIKQRLIKKVQEAVLDKWVNDPHRMDRRLLALIYLAHASDVLENAFAPLLDEQYDLATKRVRQLLDLDPEVECLKANTNEVLWAVVAAFTK

SEQ ID NO: 3 - DNA, 62 bp, Artificial Sequence
  source: /note=“Description of Artificial Sequence Synthetic primer”
tgtatgttaattgaattagcattgaggggtagattgcaactagaggcttgtggaatgagacg

SEQ ID NO: 4 - DNA, 62 bp, Artificial Sequence
  source: /note=“Description of Artificial Sequence Synthetic primer”
cgtctcattccacaagcctctagttgcaatctacccctcaatgctaattcaattaacataca

(19) United States
(12) Patent Application Publication
(10) Pub. No.: US 2011/0262464 A1
(43) Pub. Date: Oct. 27, 2011

(54) Compositions, Kits, and Methods for the Diagnosis, Prognosis, and Monitoring of Cancer Using GOLPH3

(75) Inventors: Lynda Chin, Brookline, MA (US); Kenneth Scott, Sugar Land, TX (US); Omar Kabbarah, Danville, CA (US)

(73) Assignee: DANAFARBER CANCER INSTITUTE, INC., Boston, MA (US)

(21) Appl. No.: 13/122,447

(22) PCT Filed: Oct. 5, 2009
(86) PCT No.: PCT/US2009/059526

§ 371 Date: Jul. 7, 2011

Related U.S. Application Data
(60) Provisional application No. 61/195,071, filed on Oct. 3, 2008.
(60) Provisional application No. 61/217,502, filed on Jun. 1, 2009.
(60) Provisional application No. 61/217,688, filed on Jun. 3, 2009.
(60) Provisional application No. 61/217,768, filed on Jun. 4, 2009.

(51) Int. Cl. A61K 39/395; A61K 38/00; A61P 35/00; A61K 31/7088; C12Q 1/68; G01N 33/53

(52) U.S. Cl. 424/174.1; 435/6.14; 435/6.12; 435/6.11; 435/7.1; 435/6.13; 424/178.1; 514/44 A; 514/19.3

(57) ABSTRACT

The present invention is based, in part, on the discovery that GOLPH3 plays a role in cancer, including lung, ovarian, pancreatic, liver, breast, prostate, and colon carcinomas, as well as melanoma and multiple myeloma. Accordingly, the invention relates to compositions, kits, and methods for diagnosing, prognosing, and monitoring cancer, e.g., lung, ovarian, pancreatic, liver, breast, prostate, and colon carcinomas, as well as melanoma and multiple myeloma.

### CROSS-REFERENCE TO RELATED APPLICATIONS

This application claims the benefit of priority to U.S. Provisional Application No. 61/195,071, filed on Oct. 3, 2008, U.S. Provisional Application No. 61/217,502, filed on Jun. 1, 2009, U.S. Provisional Application No. 61/217,688 filed Jun. 3, 2009 and U.S. Provisional Application No. 61/217,768, filed on Jun. 4, 2009; the contents of each application of which are hereby incorporated in their entirety.

### GOVERNMENT FUNDING

Work described herein was supported, at least in part, by National Institutes of Health (NIH) under grant RO1 CA93947 and P50 CA93683. The government may therefore have certain rights to this invention.

### BACKGROUND OF THE INVENTION

Cancer represents the phenotypic end-point of multiple genetic lesions that endow cells with a full range of biological properties required for tumorigenesis. Indeed, a hallmark genomic feature of many cancers, including, for example, B cell cancer, lung cancer, breast cancer, ovarian cancer, pancreatic cancer, and colon cancer, is the presence of numerous complex chromosome structural aberrations—including non-reciprocal translocations, amplifications and deletions.

Karyotype analyses (Johansson, B., et al. (1992) Cancer 69, 1674-81; Bardi, G., et al. (1993) Br J Cancer 67, 1106-12; Griffin, C. A., et al. (1994) Genes Chromosomes Cancer 9, 93-100; Griffin, C. A., et al. (1995) Cancer Res 55, 2394-9; Gorunova, L., et al. (1995) Genes Chromosomes Cancer 14, 259-66; Gorunova, L., et al. (1998) Genes Chromosomes Cancer 23, 81-99), chromosomal CGH and array CGH (Wolf M et al. (2004) Neoplasia 6(3)240; Kimura Y, et al. (2004) Mod. Pathol. 21 May (epub); Pinkel, et al. (1998) Nature Genetics 20:211; Solinas-Toldo, S., et al. (1996) Cancer Res 56, 3803-7; Mahlamaki, E. H., et al. (1997) Genes Chromosomes Cancer 20, 383-91; Mahlamaki, E. H., et al. (2002) Genes Chromosomes Cancer 35, 353-8; Fukushige, S., et al. (1997) Genes Chromosomes Cancer 19:161-9; Curtis, L. J., et al. (1998) Genomics 53, 42-55; Ghadimi, B. M., et al. (1999) Am J Pathol 154, 525-36; Armengol, G., et al. (2000) Cancer Genet Cytogenet 116, 133-41), fluorescence in situ hybridization (FISH) analysis (Nilsson M et al. (2004) Int J Cancer 109(3):363-9; Kawasaki K et al. (2003) Int J Mol. Med. 12(5):727-31) and loss of heterozygosity (LOH) mapping (Wang Z C et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) have identified recurrent regions of copy number change or allelic loss in various cancers.

A standard approach to identify such regions of copy number change or allelic loss associated with a specific cancer involves the identification of conserved genetic elements that are shared among samples having that particular cancer. While this approach has led to important insights into conserved amplifications or deletions that may serve as an independent predictor of cancer in a sample or subject, the presumed cancer-relevant targets relevant to the underlying disease initiation, progression and maintenance, as well as drug responsiveness in these loci harboring genomic alterations, require significant work to identify. That is, while recurrent chromosomal gains and losses have been mapped in numerous cancers, most of the presumed cancer-relevant targets in these loci remain unknown. In addition, the approach does not identify conserved genetic elements among samples derived from non-identical cancers (i.e., across multiple cancer types). Thus, it is clear that there is a need to discover the underlying genes responsible for causing and affecting cancer in order to provide improved diagnostic and prognostic systems that will guide clinical management and provide new therapeutic targets. In addition, there remains a need to identify such genes that are common across multiple cancer types.

### SUMMARY OF THE INVENTION

In order to address these deficiencies, oncogenomic analyses across multiple tumor types are described herein, which were conducted to more readily distinguish causal events governing the pathogenesis of a large fraction of human cancers. Herein, GOLPH3 was identified as the target of 5p13 amplification in numerous cancers (e.g., including at least nine solid tumor types examined with an overall frequency range of 8 to 56%). Subcellular localization with gain- and loss-of-function studies in vitro and in vivo validated GOLPH3 as a Golgi localized protein with potent oncogenic activity. Mechanistically, GOLPH3 yeast-interaction analysis, coupled with observation of knockdown-associated cell size reduction phenotype, led to confirmatory biochemical and functional studies establishing that GOLPH3 activates mTOR-S6-Kinase signaling and confers sensitivity to mTOR inhibitors (e.g., rapamycin).

mTOR, a serine/threonine protein kinase and “target of rapamycin,” serves as a primary regulator of protein synthesis and cell growth (Wullschleger et al. (2006) Cell 124: 471-484). Genetic studies in Drosophila and mice (Shima et al. (1998) Embo J. 17: 6649-6659; Montagne et al. (1999) Science 285: 2126-2129; Oldham et al. (2000) Genes Dev. 14: 2689-2694; Zhang et al. (2000) Genes Dev. 14: 2712-2724) have shown that mTOR activity can influence cell size, a key parameter governing entry into the cell cycle (Fingar et al. (2004) Oncogene 23: 3151-3171). mTOR also integrates diverse upstream signals that include amino acid and energy stress sensing to regulate cell proliferation, growth and survival (Guertin et al. (2007) Cancer Cell 12: 9-22; Yang et al. (2007) Cell Res. 17: 666-681). mTOR is present in two separate signaling complexes, mTORC1 and mTORC2, which differ in subunit composition and their sensitivity to the bacterial macrolide rapamycin. Rapamycin inhibits mTOR activity when bound to the protein raptor, leading to reduced cell growth, cell size and proliferation (Abraham et al. (1996) Annu. Rev. Immunol. 14: 483-510; Sabatini et al. (2006) Nat. Rev. Cancer 6: 729-7341 Wullschleger et al. (2006) Cell 124: 471-484).

GOLPH3 has also been shown herein to affect biosynthesis of lipid second messengers that feed into cancer signaling pathways and to impact oncogenesis through regulation by cellular growth factors (e.g., EGF). Thus, one embodiment described herein relates to GOLPH3 as a first-in-class Golgi oncoprotein linked to key signaling pathways important for cancer diagnostics, prognostics and therapeutics.

Accordingly, in one aspect, a method of assessing whether a subject is afflicted with cancer or is at risk for developing cancer is provided, the method comprising comparing the copy number of a marker in a subject sample to the normal copy number of the marker, wherein said marker comprises region 5p13 of human chromosome 5 or a fragment thereof, and wherein an altered copy number (e.g., germline and/or somatic) of the marker in the sample indicates that the subject is afflicted with cancer or at risk for developing cancer. In one embodiment, the copy number is assessed by fluorescent in situ hybridization (FISH). In another embodiment, the copy number is assessed by quantitative PCR (qPCR) or single-molecule sequencing. In still another embodiment, the normal copy number is obtained from a control sample. In yet another embodiment, the sample may be from tissue, whole blood, serum, plasma, buccal scrape, saliva, cerebrospinal fluid, urine, stool, and bone marrow.

In another aspect, the disclosure features a method of assessing whether a subject is afflicted with cancer or is at risk for developing cancer, the method comprising comparing: a) the amount, structure, subcellular localization, and/or activity of a marker in a subject sample, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and b) the normal amount, structure, subcellular localization, and/or activity of the marker, wherein a significant difference in the amount, structure, subcellular localization, and/or activity of the marker in the sample and the normal amount, structure, subcellular localization, and/or activity is an indication that the subject is afflicted with cancer or at risk for developing cancer. In one embodiment, the marker is GOLPH3. In another embodiment, the GOLPH3 marker increases cellular phospholipids, modulates retrograde trafficking by the retromer, or modulates receptory recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In certain embodiments, the method can compare the amount and/or structure and/or subcellular localization and/or the activity of the marker. In one embodiment, the amount of the marker is determined by determining the level of expression of the marker and/or by determining copy number (e.g., germline and/or somatic) of the marker (e.g., wherein the copy number is assessed by fluorescence in situ hybridization (FISH) and/or quantitative PCR (qPCR) and/or single-molecule sequencing and/or comparative genomic hybridization (CGH), such as by array CGH). In another embodiment, the normal amount, subcellular localization, structure, and/or activity is obtained from a control sample. In yet another embodiment, the sample may be from tissue, whole blood, serum, plasma, buccal scrape, saliva, cerebrospinal fluid, urine, stool, and bone marrow. In another embodiment, the level of expression of the marker in the sample is assessed by detecting the presence in the sample of a protein corresponding to the marker (e.g., wherein the presence of the protein is detected using a reagent which specifically binds with the protein, such as from the group consisting of an antibody, an antibody derivative, and an antibody fragment). In still another embodiment, the level of expression of the marker in the sample is assessed by detecting the presence in the sample of a transcribed polynucleotide or portion thereof, wherein the transcribed polynucleotide comprises the marker (e.g., an mRNA and/or a cDNA) and/or wherein the step of detecting further comprises amplifying the transcribed polynucleotide. In yet another embodiment, the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

In still another aspect, the disclosure features a method of assessing the likelihood of efficacy of an mTOR pathway inhibitor in a subject, the method comprising comparing: a) the amount, structure, subcellular localization, and/or activity of a marker in a subject sample, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and b) the normal amount, structure, subcellular localization, and/or activity of the marker, wherein a significant difference in the amount, structure, subcellular localization, and/or activity of the marker in the sample and the normal amount, structure, subcellular localization, and/or activity is an indication that an mTOR pathway inhibitor is likely to have significant efficacy in the subject. In one embodiment, the marker is GOLPH3. In another embodiment, the GOLPH3 marker increases cellular phospholipids, modulates retrograde trafficking by the retromer, or modulates receptory recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In another embodiment, the mTOR pathway inhibitor is rapamycin. The method can compare the amount and/or structure and/or subcellular localization and/or the activity of the marker. In one embodiment, the amount of the marker is determined by determining the level of expression of the marker and/or by determining copy number (e.g., germline and/or somatic) of the marker (e.g., wherein the copy number is assessed by fluorescence in situ hybridization (FISH) and/or quantitative PCR (qPCR) and/or single-molecule sequencing and/or comparative genomic hybridization (CGH), such as by array CGH). In another embodiment, the normal amount, subcellular localization, structure, and/or activity is obtained from a control sample. In yet another embodiment, the sample is may be from tissue, whole blood, serum, plasma, buccal scrape, saliva, cerebrospinal fluid, urine, stool, and bone marrow. In another embodiment, the level of expression of the marker in the sample is assessed by detecting the presence in the sample of a protein corresponding to the marker (e.g., wherein the presence of the protein is detected using a reagent which specifically binds with the protein, such as from the group consisting of an antibody, an antibody derivative, and an antibody fragment). In still another embodiment, the level of expression of the marker in the sample is assessed by detecting the presence in the sample of a transcribed polynucleotide or portion thereof, wherein the transcribed polynucleotide comprises the marker (e.g., an mRNA and/or a cDNA) and/or wherein the step of detecting further comprises amplifying the transcribed polynucleotide. In yet another embodiment, the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

In still another aspect, the disclosure features a method for monitoring the progression of cancer in a subject, the method comprising: a) detecting in a subject sample at a first point in time, the amount, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3; b) repeating step a) at a subsequent point in time; and c) comparing the amount, subcellular localization, and/or activity detected in steps a) and b), thereby monitoring the progression of cancer in the subject. In one embodiment, the marker is GOLPH3. In another embodiment, the GOLPH3 marker increases cellular phospholipids, modulates retrograde trafficking by the retromer, or modulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). The method may compare the amount and/or structure and/or subcellular localization and/or the activity of the marker. In one embodiment, the amount of the marker is determined by determining the level of expression of the marker and/or by determining copy number (e.g., germline and/or somatic) of the marker (e.g., wherein the copy number is assessed by fluorescence in situ hybridization (FISH) and/or quantitative PCR (qPCR) and/or single-molecule sequencing and/or comparative genomic hybridization (CGH), such as by array CGH). In another embodiment, the normal amount, subcellular localization, structure, and/or activity is obtained from a control sample. In yet another embodiment, the sample is may be from tissue, whole blood, serum, plasma, buccal scrape, saliva, cerebrospinal fluid, urine, stool, and bone marrow. In another embodiment, the level of expression of the marker in the sample is assessed by detecting the presence in the sample of a protein corresponding to the marker (e.g., wherein the presence of the protein is detected using a reagent which specifically binds with the protein, such as from the group consisting of an antibody, an antibody derivative, and an antibody fragment). In still another embodiment, the level of expression of the marker in the sample is assessed by detecting the presence in the sample of a transcribed polynucleotide or portion thereof, wherein the transcribed polynucleotide comprises the marker (e.g., an mRNA and/or a cDNA) and/or wherein the step of detecting further comprises amplifying the transcribed polynucleotide. In yet another embodiment, the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions. In another embodiment, the sample comprises cells obtained from the subject. In another embodiment, during the first point in time and the subsequent point in time, the subject has undergone treatment for cancer, has completed treatment for cancer, and/or is in remission.

In still another aspect, the disclosure features a method of assessing the efficacy of a test compound for inhibiting cancer in a subject, the method comprising comparing: a) the amount, subcellular localization, and/or activity of a marker in a first sample obtained from the subject and maintained in the presence of the test compound, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and b) the amount, subcellular localization, and/or activity of the marker in a second sample obtained from the subject and maintained in the absence of the test compound, wherein a significant difference in the amount, subcellular localization, and/or activity of a marker in the first sample relative to the second sample, is an indication that the test compound is efficacious for inhibiting cancer in the subject. In one embodiment, the marker is GOLPH3. In another embodiment, the GOLPH3 marker increases cellular phospholipids, modulates retrograde trafficking by the retromer, or modulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In another embodiment, the first and second samples are portions of a single sample obtained from the subject. In still another embodiment, the first and second samples are portions of pooled samples obtained from the subject.

In yet another aspect, the disclosure features a method of assessing the efficacy of a therapy for inhibiting cancer in a subject, the method comprising comparing: a) the amount, subcellular localization, and/or activity of a marker in a first sample obtained from the subject prior to providing at least a portion of the therapy to the subject, wherein the marker is selected from the group consisting of markers which reside within region 5p13 of human chromosome 5, markers which reside within the MCR consisting of 32.0 Mb to 32.8 Mb of human chromosome 5, and markers listed in Table 3, and b) the amount, subcellular localization, and/or activity of the marker in a second sample obtained from the subject following provision of the portion of the therapy, wherein a significant difference in the amount, subcellular localization, and/or activity of a marker in the first sample relative to the second sample, is an indication that the therapy is efficacious for inhibiting cancer in the subject. In one embodiment, the marker is GOLPH3. In another embodiment, the GOLPH3 marker increases cellular phospholipids, modulates retrograde trafficking by the retromer, or modulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF).

In another aspect, the disclosure features a method of selecting a composition capable of modulating cancer, the method comprising: a) obtaining a sample comprising cancer cells; b) contacting said cells with a test compound; and c) determining the ability of the test compound to modulate the amount, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3, thereby identifying a modulator of cancer. In one embodiment, the marker is GOLPH3. In another embodiment, the GOLPH3 marker increases cellular phospholipids, modulates retrograde trafficking by the retromer, or modulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In still another embodiment, said cells are isolated from an animal model of cancer. In yet another embodiment, said cells are from a cancer cell line. In another embodiment, said cells are from a subject suffering from cancer. In another embodiment, said cells may be from lung carcinoma, ovarian carcinoma, melanoma, breast carcinoma, colon carcinoma, multiple myeloma, prostate carcinoma, pancreatic carcinoma, and liver carcinoma cell lines. In still another embodiment, the method further comprises administering the test compound to an animal model of cancer. In yet another embodiment, the modulator changes the subcellular localization or inhibits the amount and/or activity of a gene or protein corresponding to GOLPH3.

In another aspect, the disclosure features a method of selecting a composition capable of modulating cancer, the method comprising: a) contacting a marker with a test compound, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and b) determining the ability of the test compound to modulate the amount, subcellular localization, and/or activity of a marker which resides in the MCR, thereby identifying a composition capable of modulating cancer. In one embodiment, the marker is GOLPH3. In another embodiment, the GOLPH3 marker increases cellular phospholipids, modulates retrograde trafficking by the retromer, or moedulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In another embodiment, the method further comprises administering the test compound to an animal model of cancer. In still another embodiment, the modulator changes the subcellular localization or inhibits the amount and/or activity of a gene or protein corresponding to GOLPH3.

In still another aspect, the disclosure features a method of treating a subject afflicted with cancer comprising administering to the subject a compound which changes the subcellular localization of or modulates the amount and/or activity of a gene or protein corresponding to a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3. In one embodiment, the marker is GOLPH3. In another embodiment, the GOLPH3 marker increases cellular phospholipids, modulates retrograde trafficking by the retromer, or modulates receptor recycling. In still another embodiment, the cellular phospholipids may be PIP₂ and/or PA. In yet another embodiment, the GOLPH3 modulates the PI3K pathway. In another embodiment, the GOLPH3 is phosphorylated by ARF4 and/or GOLPH3 phosphorylation levels are reduced after exposure of the subject sample to cellular growth factors (e.g., EGF) and/or GOLPH3 translocates from the Golgi to the plasma membrane after exposure of the subject sample to cellular growth factors (e.g., EGF). In one embodiment, said compound is administered in a pharmaceutically acceptable formulation. In another embodiment, said compound is an antibody or an antigen binding fragment thereof, which specifically binds to a protein corresponding to said marker (e.g., wherein the antibody is conjugated to a toxin and/or a chemotherapeutic agent). In yet another embodiment, said compound is an RNA interfering agent which inhibits expression of a gene corresponding to said marker (e.g., an siRNA molecule or an shRNA molecule). In another embodiment, said compound is an antisense oligonucleotide complementary to a gene corresponding to said marker. In still another embodiment, said compound is a peptide or peptidomimetic. In yet another embodiment, said compound is a small molecule which inhibits activity of said marker (e.g., a small molecule that inhibits a protein-protein interaction between a marker and a target protein). In yet another embodiment, said compound is an aptamer which inhibits expression or activity of said marker.

In another aspect, the disclosure features various kits. In one embodiment, the disclosure features a kit for assessing whether a subject is afflicted with cancer, the kit comprising a reagent for assessing the copy number of a marker, wherein the marker comprises region 5p13 of human chromosome 5 or a fragment thereof. In another embodiment, the disclosure features a kit for assessing the ability of a compound to inhibit cancer, the kit comprising a reagent for assessing the amount, structure, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3. In still another embodiment, the disclosure features a kit for assessing whether a subject is afflicted with cancer, the kit comprising a reagent for assessing the amount, structure, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3. In yet another embodiment, the disclosure features a kit for assessing the presence of human cancer cells, the kit comprising an antibody or fragment thereof, wherein the antibody or fragment thereof specifically binds with a protein corresponding to a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3. In another embodiment, the disclosure features a kit for assessing the presence of cancer cells, the kit comprising a nucleic acid probe wherein the probe specifically binds with a transcribed polynucleotide corresponding to a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3. In another embodiment, the marker of any of the kits is GOLPH3. In another embodiment, the marker of any of the kits is GOLPH3 and wherein GOLPH3 increases cellular phospholipids, modulates retrograde trafficking by the retromer, or modulates receptor recycling (e.g., wherein the cellular phospholipids may be PIP₂ and/or PA). In still another embodiment, the marker of any of the kits is GOLPH3 and wherein GOLPH3 modulates the PI3K pathway. In yet another embodiment, the marker of any of the kits is GOLPH3 and wherein GOLPH3 is phosphorylated by ARF4. In another embodiment, the marker of any of the kits is GOLPH3 and wherein GOLPH3 phosphorylation levels are reduced after exposure of the subject sample to EGF. In another embodiment, the marker of any of the kits is GOLPH3 and wherein GOLPH3 translocates from the Golgi after exposure of the subject sample to EGF.

### BRIEF DESCRIPTION OF THE DRAWINGS

FIGS. 1A-1E show genomic characterization of 5p13 amplification. FIG. 1A shows an array-CGH heat map detailing GOLPH3 amplification at 5p13 in representative tumor specimens and cell lines from malignant melanoma (MeI), colon adenocarcinoma (CRC) and non-small cell lung cancer (NSCLC). Regions of genomic amplification and deletion are denoted in red and blue, respectively. Mbs=position on chromosome 5 in megabases. FIG. 1B shows a histogram summary of copy number status at 5p13 by TMA-FISH analysis of 307 tumor cores of the indicated tumor types. CRC=colon adenocarcinoma; NSCLC=small cell lung cancer; MM=multiple myeloma; OV=ovarian carcinoma; PDAC=pancreatic ductal adenocarcinoma. FIG. 1C shows the minimum common region of the 5p13 amplicon defined by array-CGH from one representative tumor (melanoma C27) with focal amplification. FIG. 1D shows delimitation of chromosome 5p13 amplicon boundaries by genomic qPCR using four informative cell line and tumor specimens. FIG. 1E shows a heat map depiction of Affymetrix expression data for NSCLC 5p13 amplified (AMP) and normal specimens. *=significant correlation after bonferroni correction for multiple testing.

FIGS. 2A-2E shows functional validation data of GOLPH3. FIG. 2A shows the results of the indicated cell lines treated with non-targeting siRNA (siNT) or individual siRNAs against GOLPH3 (si #1-si #4) for effect on anchorage-independent growth in soft agar (left panels) and cell proliferation (right panels). Bars indicate ±S.D. FIG. 2B shows the results of A549 parental cells and those expressing either wild type (WT) or siRNA resistant GOLPH3 (siRES) treated with either non-targeting siRNA (siNT) or siRNA against GOLPH3 (siGOLPH3) for effect on cell proliferation. Bars indicate ±S.D. Shown are endpoint values for day 5. FIG. 2C shows the results of primary Ink4a/Arf-deficient MEFs transfected with the indicated vectors expressing HRAS^{V12}, MYC and GOLPH3. Vec=LacZ vector control; bars indicate ±S.D.; Two-tailed t-test: HRAS^{V12}+GOLPH3 vs. HRAS^{V12}+Vec, p=0.0018. FIG. 2D shows the results of TERT-immortalized human melanocytes (HMEL) expressing activated BRAF^{V600E} transduced with either GOLPH3 or SUB1 to assay for effect on anchorage-independent growth in soft agar. Bars indicate ±S.D.; Two-tailed t-test for colony number: EV vs. GOLPH3, p=0.0020; EV vs. SUB1, p=0.3739. FIG. 2E shows the results of the indicated cell lines transduced with GOLPH3 to assay for effect on growth of mouse xenograft tumors.

FIGS. 3A-3D show that GOLPH3 interacts with VPS35 and influences cell size. FIG. 3A shows GOLPH3 positive endosome-like structures (arrows) in both 1205LU melanoma cells stably-expressing GOLPH3 that were co-immunostained for HA (GOLPH3^{HA}; green) and TGN46 (Golgi marker; red) (top panel) and A549 cells co-immunostained for GOLPH3 (green) and TGN46 (Golgi marker; red) (bottom panel). DNA was labeled with DAPI. FIG. 3B shows immunoblotting results of isolated proteins immunoprecipitated (IP) with anti-HA (left panel) or anti-V5 (right panel) for immunoblotting with the indicated antibodies from 239T cells transiently expressing the indicated constructs. NS=non-specific band. FIG. 3C shows GOLPH3 positive co-staining at endosome structures (arrows) in A549 cells co-immunostained for GOLPH3 (green) and VPS35 (red). DNA was labeled with DAPI (blue). FIG. 3D shows Automated Quantitative Analysis (AQUA®) of phospho-S6K^{Thr389} (red) in two representative lung adenocarcinomas. Cytokeratin (green) defines tumor and non-nuclear compartments. FISH ratio=5p13:reference ratio as determined by FISH on consecutive TMA sections. Magnification=20×.

FIGS. 4A-4E show that GOLPH3 modulates phosphorylation status of mTOR substrates. FIG. 4A shows representative flow histograms for A549 cells treated with non-targeting (siNT, blue), siRNA against GOLPH3 (siGOLPH3, left panel, green) or rapamycin (Rap, middle panel, green). Peak FSC-H is indicated in the histograms and the right panel shows mean FSC-H for multiple experiments (n=3); Bars indicate ±S.D. FIG. 4B shows protein lysates extracted from 1205LU (left panel), A549 (middle panel) and HMEL-tet-GOLPH3 (right panel; with or without doxycycline (DOX)) cells expressing GOLPH3 immunoblotted with the indicated antibodies. FIG. 4C shows the results of HMEL-tet-GOLPH3 cells that were serum depleted and propagated with or without doxycycline (DOX), followed by treatment with or without EGF for 30 min for immunoblot analysis with the indicated antibodies. FIGS. 4D-4E shows the results of A549 (FIG. 4D) and CRL-5889 (FIG. 4E) cells that were serum depleted and treated with either non-targeting (siNT) or siRNA against GOLPH3 (siGOLPH3), followed by growth factor stimulation with EGF for immunoblot analysis with the indicated antibodies.

FIGS. 5A-5C shows that in vivo GOLPH3 growth advantage is abrogated by treatment with rapamycin. Mice harboring tumors of the melanoma cell lines WM239A (FIG. 5A) and 1205LU (FIG. 5B) transduced with empty vector (EV; left panels) or GOLPH3 (right panels) were treated with vehicle or rapamycin (6.0 mg/kg) at two-day increments following treatment onset (tumor baseline volume ˜100 mm³). Growth curves were plotted as mean change in tumor volume relative to baseline starting volume for each group. Bars indicate ±S.E.M. for biological replicates. % TGI indicates percent tumor growth inhibition at time course endpoint. FIG. 5C summarizes data from the rapamycin treatment xenograft studies shown in FIGS. 5A-5B at the same time point (day 8, post 4 doses). Veh indicates vehicle; Rap indicates rapamycin; % TGI indicates percent tumor growth inhibition. 1205LU-GOLPH3 tumors treated with vehicle grew 2.5× in size during the 8 days of treatment. In comparison, WM239A-GOLPH3 tumors grew 5.8× in size during the same period of 8 days. The % TGI in these two cohorts of tumors was similar, at 81.9% and 80.9% respectively, indicating that growth rate did not impact on the response to rapamycin.

FIG. 6 shows the results of genomic identification of the 5p13 amplicon. Representative images of TMA-FISH analysis are shown for 5p13 amplification in tumor core specimens: (i) benign compound nevus of right waist, (ii) malignant melanoma of left heel, (iii) normal lung and (iv) grade II lung adenocarcinoma. Regions of 5p13 and centromere-specific ploidy reference are indicated by green and red, respectively.

FIGS. 7A-7G show additional results of immunoblot and anchorage-independence assays for GOLPH3. FIG. 7A shows immunoblot analysis of GOLPH3 expression in 5p13 amplified (AMP) and normal (NL) melanoma and NSCLC cell lines. NHM=normal human melanocytes. FIG. 7B shows confirmation of GOLPH3 knockdown in NSCLC CRL-5889 (top panel) and melanoma 1205LU (bottom panel) for the anchorage-independent growth and proliferation assays presented in FIG. 2A. GOLPH3 knockdown was performed using the indicated siRNAs (si #1-si #4). siNT=non-targeting siRNA control; P=parental A549 lysate. FIG. 7C shows confirmation of GOLPH3 expression in GOLPH3-transduced melanoma 1205LU used in FIG. 7D and xenograft assays. 1205LU lysates were extracted from cells used for GOLPH3 gain-of-function experiment presented in FIG. 4B and are therefore presented in both figures. FIG. 7D shows that GOLPH3 enhances anchorage-independent growth of human melanoma 1205LU cells (without 5p13 amplification). EV indicates empty vector; Bars indicate ±S.D.; Two-tailed t-test for colony number, p=0.0003. FIGS. 7E-7F show confirmation of GOLPH3 expression in the xenograft assays using GOLPH3-transduced melanoma WM239A cells (FIG. 7E) and GOLPH3-transduced NSCLC A549 cells (FIG. 7F). Whole cell lysates were immunoblotted with the indicated antibodies. FIG. 7G shows confirmation of mTOR inhibition by rapamycin. Whole cell lysates extracted from representative empty vector (EV; n=3) or GOLPH3 (n=5) WM239A xenograft tumors that were harvested from mice treated with (+) or without (−) rapamycin were immunoblotted with the indicated antibodies.

FIGS. 8A-8C show the results of yeast two-hybrid screening for GOLPH3-interacting proteins. FIG. 8A shows that yeast two-hybrid screening identified VPS35 as a GOLPH3-interacting protein. The yeast reporter strain (AH109) co-expressing VPS35 (prey) with either GOLPH3 (+) or empty vector (−) plated on SC-Leucine-Histidine-Adenine+XaGal (SC-L-H-A+XαGAL) reporter plates confirm reporter activation and GOLPH3 bait-dependency. FIG. 8B shows controls for the yeast two-hybrid screen. Negative control (−), AH109 expressing pGBKT7-Lam (bait; TRP) and pGADT7-T (prey, LEU); positive control (+), AH109 expressing pGBKT7-p53 (bait, TRP) and GADT7-T (prey, LEU); positive control (++), AH109 expressing pGBKT7 (empty vector, TRP) and pCL1 (GAL4 activation domain, LEU); bait-independent false positive control (++-bait) AH109 expressing pCL1 alone. Strains from SC-Leucine (SC-L) were replica plated to SC-Leucine-Trptophan (SC-LT) to confirm presence of bait and prey and to SC-Leucine-Histidine-Adenine+XaGal (SC-L-H-A+XαGAL) to confirm reporter activation through the cells ability to grow and express the a-galactosidase reporter (indicated by blue appearance). Controls were used as comparison for positive clone selection and bait-dependency testing. FIG. 8C shows the interaction of endogenous GOLPH3 with VPS35 as determined by co-immunoprecipitation analysis. NSCLC A549 protein extracts were immunoprecipitated (IP) with either control (C) or anti-GOLPH3 (G3) mouse serum for immunoblotting with the indicated antibodies.

FIG. 9 shows the results of Western blot analysis for GOLPH3-dependent changes in mTOR substrates and other MAPK-/PI3K-relevant proteins. The indicated cell lines were either transduced with empty vector (EV) or GOLPH3 (two left panels, over-expression) or transfected with non-targeting siRNA (siNT) or siRNA against GOLPH3 (siGOLPH3) (two right panels, knockdown). Whole cell lysates were immunoblotted with the indicated antibodies.

FIGS. 10A-10B show the results of endpoint analysis for rapamycin treatment xenograft studies indicated by endpoint tumor volume measurements for tumors presented in FIG. 5. Values are plotted as proportion of dose 4 or dose 6 endpoint tumor volumes over respective baseline starting volume for WM239A (FIG. 10A) and 1205LU (FIG. 10B) xenografts, respectively. Rap indicates rapamycin; Two-tailed t-test for rapamycin treated WM239A and 1205LU EV vs. GOLPH3 xenografts, p=0.0677 and p=0.0268, respectively.

FIG. 11A-11C show reduction of lipid second messenger biosynthesis upon GOLPH3 depletion. FIG. 11A shows that depletion of GOLPH3 reduces cell migration, possibly through either an mTOR- or phospholipid-mediated pathway. FIG. 11B shows that depletion of GOLPH3 reduces in vivo basal and growth factor simulated biosynthesis of lipid second messengers that feed into cancer signaling pathways. FIG. 11C shows quantitative results of the data presented in FIG. 11B.

FIG. 12A-12D show that growth factor signaling causes GOLPH3 mis-localization via ARF4. FIG. 12A shows that ARF4, a GTPase identified herein as a GOLPH3-interacting protein, co-localizes with GOLPH3 in the Golgi. FIG. 12B shows that GTP-mediated phosphorylation of GOLPH3 is required for GOLPH3 localization to the Golgi and that depletion of ARF4 causes mislocalization of GOLPH3 from the Golgi to other parts of the cell, including the cell periphery. FIG. 12C shows the kinetics of EGF receptor phosphorylation upon stimulating with EGF in a representative cancer cell line (A549). FIG. 12D shows that growth factor signaling (e.g., EGF) causes redistribution of GOLPH3 from the Golgi to other parts of the cell, suggesting that growth factors might do so by decreasing GOLPH3 phosphorylation by ARF4 since ARF4 is known to relocalize to the plasma membrane upon EGF administration.

### DETAILED DESCRIPTION OF THE INVENTION

The invention is based, in part, on the discovery that GOLPH3, a Golgi localized protein, was identified as a novel oncogene from within a conserved 5p13 amplification in numerous cancers (e.g., at least nine solid tumor types). GOLPH3 yeast-interaction analysis, coupled with observation of knockdown-associated cell size reduction phenotype, led to confirmatory biochemical and functional studies establishing that GOLPH3 activates mTOR-S6-Kinase signaling and confers sensitivity to mTOR inhibitors (e.g., rapamycin).

GOLPH3 has also been shown herein to affect biosynthesis of lipid second messengers that feed into cancer signaling pathways and to impact oncogenesis through regulation by cellular growth factors (e.g., EGF). GOLPH3 polypeptides and fragments thereof, e.g., biologically active or antigenic fragments thereof, are provided, as reagents or targets in assays applicable to diagnosis of cancer, e.g., lung, ovarian, pancreatic, liver, breast, prostate, and colon carcinomas, as well as melanoma and multiple myeloma. In particular, the methods and compositions of the present disclosure relate to detection of expression and/or activity of a GOLPH3 gene or fragment thereof, e.g., biologically active fragments thereof, as well as to the detection of expression and/or activity of gene products or fragments thereof encoded by the GOLPH3 gene, e.g., biologically active fragments thereof. The methods and compositions of the present disclosure can utilize the GOLPH3 gene or gene sequence or fragments thereof, as well as gene products of the GOLPH3 gene and/or fragments thereof, e.g., antibodies which specifically bind to such GOLPH3 gene products.

In one aspect, methods are provided for detecting the presence, absence, stage, and other characteristics of cancers, e.g., lung, ovarian, pancreatic, liver, breast, prostate, and colon carcinomas, as well as melanoma and multiple myeloma, in a sample that are relevant to prognosis, diagnosis, monitoring, and characterization in a patient.

The disclosure also features compositions of matter, including antibodies (e.g., antibodies which specifically bind to any one of the polypeptides described herein) as well as fusion polypeptides, including all or a fragment of a polypeptide described herein.

### I. DEFINITIONS

The articles “a” and “an” are used herein to refer to one or to more than one (i.e. to at least one) of the grammatical object of the article. By way of example, “an element” means one element or more than one element.

The term “altered amount” of a marker or “altered level” of a marker refers to increased or decreased copy number (e.g., germline and/or somatic) of a marker or chromosomal region, e.g., MCR, and/or increased or decreased expression level of a particular marker gene or genes in a cancer sample, as compared to the expression level or copy number of the marker in a control sample. The term “altered amount” of a marker also includes an increased or decreased protein level of a marker in a sample, e.g., a cancer sample, as compared to the protein level of the marker in a normal, control sample. Furthermore, an altered amount of a marker may be determined by detecting the methylation status of a marker, as described herein, which may affect the expression or activity of a marker.

The amount of a marker, e.g., expression or copy number of a marker or MCR, or protein level of a marker, in a subject is “significantly” higher or lower than the normal amount of a marker or MCR, if the amount of the marker is greater or less, respectively, than the normal level by an amount greater than the standard error of the assay employed to assess amount, and preferably at least twice, and more preferably three, four, five, ten or more times that amount. Alternately, the amount of the marker or MCR in the subject can be considered “significantly” higher or lower than the normal amount if the amount is at least about two, and preferably at least about three, four, or five times, higher or lower, respectively, than the normal amount of the marker or MCR.

The term “altered level of expression” of a marker or MCR refers to an expression level or copy number of a marker in a test sample e.g., a sample derived from a patient suffering from cancer, that is greater or less than the standard error of the assay employed to assess expression or copy number, and is preferably at least twice, and more preferably three, four, five or ten or more times the expression level or copy number of the marker or MCR in a control sample (e.g., sample from a healthy subjects not having the associated disease) and preferably, the average expression level or copy number of the marker or MCR in several control samples. The altered level of expression is greater or less than the standard error of the assay employed to assess expression or copy number, and is preferably at least twice, and more preferably three, four, five or ten or more times the expression level or copy number of the marker or MCR in a control sample (e.g., sample from a healthy subjects not having the associated disease) and preferably, the average expression level or copy number of the marker or MCR in several control samples.

The term “altered activity” of a marker refers to an activity of a marker which is increased or decreased in a disease state, e.g., in a cancer sample, as compared to the activity of the marker in a normal, control sample. Altered activity of a marker may be the result of, for example, altered expression of the marker, altered protein level of the marker, altered structure of the marker, or, e.g., an altered interaction with other proteins involved in the same or different pathway as the marker or altered interaction with transcriptional activators or inhibitors, or altered methylation status.

The term “altered structure” of a marker refers to the presence of mutations or allelic variants within the marker gene or maker protein, e.g., mutations which affect expression or activity of the marker, as compared to the normal or wild-type gene or protein. For example, mutations include, but are not limited to substitutions, deletions, or addition mutations. Mutations may be present in the coding or non-coding region of the marker.

The term “altered subcellular localization” of a marker refers to the mislocalization of the marker within a cell relative to the normal localization within the cell (e.g., within a healthy and/or wild-type cell. An indication of normal localization of the marker can be determined through an analysis of subcellular localization motifs known in the field that are harbored by marker polypeptides.

Unless otherwise specified here within, the terms “antibody” and “antibodies” broadly encompass naturally-occurring forms of antibodies (e.g. IgG, IgA, IgM, IgE) and recombinant antibodies such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., GOLPH3 polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov, S. M., et al. (1995) Human Antibodies and Hybridomas 6:93-101) and use of a cysteine residue, a marker peptide and a C-terminal polyhistidine tag to make bivalent and biotinylated scFv polypeptides (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to GOLPH3 polypeptides or fragments thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

The term “body fluid” refers to fluids that are excreted or secreted from the body as well as fluid that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenström's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like.

The term “cellular growth factors” refers to cellular growth factors well known in the art, including, e.g., EGF, FGF, TGF-α, TGF-β, PDGF, IGF-1, IGF-2 BNDF, BMP, GGRP, GDNF, GGF, HGF, KGF, mytotrophin, NGF, OSM, somatotrophin, and VEGF.

The term “cellular phospholipids” encompasses cellular phospholipids well known in the art, including, e.g., PIP2, PIP3, IP3, DAG, and PA).

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The “copy number of a gene” or the “copy number of a marker” refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include chagnes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes includechanges at one or more genomic loci, wherein said one or more genomic loci are not accounted for by the number of copies in germline DNA of a control (e.g., copy number in germline DNA for the same subject as that from which the somatic DNA and corresponding copy number were determined).

The “normal” copy number (e.g., germline and/or somatic) of a marker or MCR or “normal” level of expression of a marker is the level of expression, copy number of the marker, or copy number of the MCR, in a biological sample, e.g., a sample containing tissue, whole blood, serum, plasma, buccal scrape, saliva, cerebrospinal fluid, urine, stool, and bone marrow, from a subject, e.g., a human, not afflicted with cancer.

As used herein, the term “diagnostic marker” includes markers listed herein which are useful in the diagnosis of cancer, e.g., over- or under-activity, emergence, expression, growth, remission, recurrence or resistance of tumors before, during or after therapy. The predictive functions of the marker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression (e.g., by ISH, Northern Blot, or qPCR), increased or decreased protein level (e.g., by IHC), or increased or decreased activity (determined by, for example, modulation of a pathway in which the marker is involved), e.g., in more than about 5%, 6%, 7%, 8%, 9%, 10%, 11%, 12%, 13%, 14%, 15%, 20%, 25%, or more of human cancers types or cancer samples; (2) its presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its presence or absence in clinical subset of patients with cancer (e.g., those responding to a particular therapy or those developing resistance).

Diagnostic markers also include “surrogate markers,” e.g., markers which are indirect markers of cancer progression.

A molecule is “fixed” or “affixed” to a substrate if it is covalently or non-covalently associated with the substrate such that the substrate can be rinsed with a fluid (e.g. standard saline citrate, pH 7.4) without a substantial fraction of the molecule dissociating from the substrate.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

The term “humanized antibody”, as used herein, is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

An “inducible” promoter is a nucleotide sequence which, when operably linked with a polynucleotide which encodes or specifies a gene product, causes the gene product to be produced in a living human cell substantially only when an inducer which corresponds to the promoter is present in the cell.

As used herein, the term “inhibit” includes the decrease, limitation, or blockage, of, for example a particular action, function, or interaction.

Cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

As used herein, the term “interaction”, when referring to an interaction between two molecules, refers to the physical contact (e.g., binding) of the molecules with one another. Generally, such an interaction results in an activity (which produces a biological effect) of one or both of said molecules.

An “isolated antibody”, as used herein, is intended to refer to an antibody that is substantially free of other antibodies having different antigenic specificities (e.g., an isolated antibody that specifically binds GOLPH3 polypeptide or a fragment thereof is substantially free of antibodies that specifically bind antigens other than a GOLPH3 polypeptide or a fragment thereof). Moreover, an isolated antibody may be substantially free of other cellular material and/or chemicals.

As used herein, an “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of GOLPH3 polypeptide or fragment thereof, in which the protein is separated from cellular components of the cells from which it is isolated or recombinantly produced. In one embodiment, the language “substantially free of cellular material” includes preparations of GOLPH3 protein or fragment thereof, having less than about 30% (by dry weight) of non-GOLPH3 protein (also referred to herein as a “contaminating protein”), more preferably less than about 20% of non-GOLPH3 protein, still more preferably less than about 10% of non-GOLPH3 protein, and most preferably less than about 5% non-GOLPH3 protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g. a package or container) comprising at least one reagent, e.g. a probe, for specifically detecting the expression of a marker of the invention. The kit may be promoted, distributed, or sold as a unit for performing the methods of the present invention. The kit may comprise one or more reagents necessary to express a marker of the invention (e.g., GOLPH3). In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit can be included.

A “marker” is a gene whose altered level of expression in a tissue or cell from its expression level in normal or healthy tissue or cell is associated with a disease state, such as cancer. A “marker nucleic acid” is a nucleic acid (e.g., mRNA, cDNA) encoded by or corresponding to a marker of the invention. Such marker nucleic acids include DNA (e.g., cDNA) comprising the entire or a partial sequence of any of the nucleic acid sequences set forth in the Sequence Listing or the complement of such a sequence. The marker nucleic acids also include RNA comprising the entire or a partial sequence of any of the nucleic acid sequences set forth in the Sequence Listing or the complement of such a sequence, wherein all thymidine residues are replaced with uridine residues. A “marker protein” is a protein encoded by or corresponding to a marker of the invention. A marker protein comprises the entire or a partial sequence of any of the sequences set forth in the Sequence Listing. The terms “protein” and “polypeptide” are used interchangeably.

A “minimal common region (MCR),” as used herein, refers to a contiguous chromosomal region which displays either gain and amplification (increased copy number) or loss and deletion (decreased copy number) in the genome of a cancer. An MCR includes at least one nucleic acid sequence which has increased or decreased copy number and which is associated with a cancer.

The “normal” level of expression of a marker is the level of expression of the marker in cells of a subject, e.g., a human patient, not afflicted with a cancer, e.g., lung, ovarian, pancreatic, liver, breast, prostate, and colon carcinomas, as well as melanoma and multiple myeloma. An “over-expression” or “significantly higher level of expression” of a marker refers to an expression level in a test sample that is greater than the standard error of the assay employed to assess expression, and is preferably at least twice, and more preferably three, four, five or ten times the expression level of the marker in a control sample (e.g., sample from a healthy subjects not having the marker associated disease) and preferably, the average expression level of the marker in several control samples. A “significantly lower level of expression” of a marker refers to an expression level in a test sample that is at least twice, and more preferably three, four, five or ten times lower than the expression level of the marker in a control sample (e.g., sample from a healthy subject not having the marker associated disease) and preferably, the average expression level of the marker in several control samples.

An “overexpression” or “significantly higher level of expression or copy number” of a marker or MCR refers to an expression level or copy number in a test sample that is greater than the standard error of the assay employed to assess expression or copy number, and is preferably at least twice, and more preferably three, four, five or ten or more times the expression level or copy number of the marker or MCR in a control sample (e.g., sample from a healthy subject not afflicted with cancer) and preferably, the average expression level or copy number of the marker or MCR in several control samples.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

An “RNA interfering agent” as used herein, is defined as any agent which interferes with or inhibits expression of a target gene, e.g., a marker of the invention, by RNA interference (RNAi). Such RNA interfering agents include, but are not limited to, nucleic acid molecules including RNA molecules which are homologous to the target gene, e.g., a marker of the invention, or a fragment thereof, short interfering RNA (siRNA), and small molecules which interfere with or inhibit expression of a target gene by RNA interference (RNAi).

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” or “inhibition of marker gene expression” includes any decrease in expression or protein activity or level of the target gene (e.g., a marker gene of the invention) or protein encoded by the target gene, e.g., a marker protein of the invention. The decrease may be of at least 30%, 40%, 50%, 60%, 70%, 80%, 90%, 95% or 99% or more as compared to the expression of a target gene or the activity or level of the protein encoded by a target gene which has not been targeted by an RNA interfering agent.

“Short interfering RNA” (siRNA), also referred to herein as “small interfering RNA” is defined as an agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated be reference herein).

RNA interfering agents, e.g., siRNA molecules, may be administered to a patient having or at risk for having cancer, to inhibit expression of a marker gene of the invention, e.g., a marker gene which is overexpressed in cancer (such as the markers listed in Table 3) and thereby treat, prevent, or inhibit cancer in the subject.

A “constitutive” promoter is a nucleotide sequence which, when operably linked with a polynucleotide which encodes or specifies a gene product, causes the gene product to be produced in a living human cell under most or all physiological conditions of the cell.

As used herein, “subject” refers to any healthy animal, mammal or human, or any animal, mammal or human afflicted with a cancer, e.g., lung, ovarian, pancreatic, liver, breast, prostate, and colon carcinomas, as well as melanoma and multiple myeloma. The term “subject” is interchangeable with “patient”.

The language “substantially free of chemical precursors or other chemicals” includes preparations of antibody, polypeptide, peptide or fusion protein in which the protein is separated from chemical precursors or other chemicals which are involved in the synthesis of the protein. In one embodiment, the language “substantially free of chemical precursors or other chemicals” includes preparations of antibody, polypeptide, peptide or fusion protein having less than about 30% (by dry weight) of chemical precursors or non-antibody, polypeptide, peptide or fusion protein chemicals, more preferably less than about 20% chemical precursors or non-antibody, polypeptide, peptide or fusion protein chemicals, still more preferably less than about 10% chemical precursors or non-antibody, polypeptide, peptide or fusion protein chemicals, and most preferably less than about 5% chemical precursors or non-antibody, polypeptide, peptide or fusion protein chemicals.

A “tissue-specific” promoter is a nucleotide sequence which, when operably linked with a polynucleotide which encodes or specifies a gene product, causes the gene product to be produced in a living human cell substantially only if the cell is a cell of the tissue type corresponding to the promoter.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a marker of the invention and normal post-transcriptional processing (e.g. splicing), if any, of the RNA transcript, and reverse transcription of the RNA transcript.

An “underexpression” or “significantly lower level of expression or copy number” of a marker or MCR refers to an expression level or copy number in a test sample that is greater than the standard error of the assay employed to assess expression or copy number, but is preferably at least twice, and more preferably three, four, five or ten or more times less than the expression level or copy number of the marker or MCR in a control sample (e.g., sample from a healthy subject not afflicted with cancer) and preferably, the average expression level or copy number of the marker or MCR in several control samples.

As used herein, the term “vector” refers to a nucleic acid capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, |
| | CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, |
| | TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, |
| | TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA coding for a fusion protein or polypeptide of the invention (or any portion thereof) can be used to derive the fusion protein or polypeptide amino acid sequence, using the genetic code to translate the DNA or RNA into an amino acid sequence. Likewise, for fusion protein or polypeptide amino acid sequence, corresponding nucleotide sequences that can encode the fusion protein or polypeptide can be deduced from the genetic code (which, because of its redundancy, will produce multiple nucleic acid sequences for any given amino acid sequence). Thus, description and/or disclosure herein of a nucleotide sequence which encodes a fusion protein or polypeptide should be considered to also include description and/or disclosure of the amino acid sequence encoded by the nucleotide sequence. Similarly, description and/or disclosure of a fusion protein or polypeptide amino acid sequence herein should be considered to also include description and/or disclosure of all possible nucleotide sequences that can encode the amino acid sequence.

### I. Description

The present disclosure relates to methods and compositions for the diagnosis, prognosis, and monitoring of cancers, e.g., lung, ovarian, pancreatic, liver, breast, prostate, and colon carcinomas, as well as melanoma and multiple myeloma cancer.

In particular, the methods and compositions of the present disclosure relate to detection of expression and/or activity of a gene referred to herein as the GOLPH3 gene or a fragment thereof, e.g., a biologically active fragment thereof, as well as to the detection of expression and/or activity of gene products encoded by the GOLPH3 gene (i.e., a “GOLPH3 gene product”) or fragments thereof, e.g., biologically active fragments thereof. The methods and compositions of the present disclosure can utilize the GOLPH3 gene or gene sequence or fragments thereof, as well as gene products of the GOLPH3 gene, e.g., antibodies which specifically bind to such GOLPH3 gene products, or fragments thereof. Sequences, splice variants, and structures of GOLPH3 gene and gene products have been described in the art. See, for example, the Gene Cards.com website and Bell et al., (2001) J Biol Chem 276: 5152-5165. GOLPH3 gene and gene products from many species are known and include, for example, chimpanzee GOLPH3 (NCBI Accession XM_{—}517830.2), rat GOLPH3 (NCBI Accession NM_{—}023977.2), mouse GOLPH3 (NM_{—}02567.3), chicken GOLPH3 (XM_{—}424995.2), and human GOLPH3 (NM_{—}022130 and NP_{—}071413). Human GOLPH3 sequences include those listed below.

GOLPH3 coding nucleic acid sequence:

| | |
|---|---|
| 1 | atgacctcgc tgacccagcg cagctccggc ctggtgcagc |
| | ggcgcaccga ggcctcccgc |
| 61 | aacgccgccg acaaggagcg ggcggcgggc ggcggcgccg |
| | gcagcagcga ggacgacgcg |
| 121 | cagagccgcc gcgacgagca ggacgacgac gacaagggcg |
| | actccaagga aacgcggctg |
| 181 | accctgatgg aggaagtgct cctgctgggc ctcaaggacc |
| | gcgagggtta cacatcattt |
| 241 | tggaatgact gtatatcatc tggattacgt ggctgtatgt |
| | taattgaatt agcattgaga |
| 301 | ggaaggttac aactagaggc ttgtggaatg agacgtaaaa |
| | gtctattaac aagaaaggta |
| 361 | atctgtaagt cagatgctcc aacaggggat gttcttcttg |
| | atgaagctct gaagcatgtt |
| 421 | aaggaaactc agcctccaga aacggtccag aactggattg |
| | aattacttag tggtgagaca |
| 481 | tggaatccat taaaattgca ttatcagtta agaaatgtac |
| | gggaacgatt agctaaaaac |
| 541 | ctggtggaaa agggtgtatt gacaacagag aaacagaact |
| | tcctactttt tgacatgaca |
| 601 | acacatcccc tcaccaataa caacattaag cagcgcctca |
| | tcaagaaagt acaggaagcc |
| 661 | gttcttgaca aatgggtgaa tgaccctcac cgcatggaca |
| | ggcgcttgct ggccctcatt |
| 721 | tacctggctc atgcctcgga cgtcctggag aatgcttttg |
| | ctcctcttct ggacgagcag |
| 781 | tatgatttgg ctaccaagag agtgcggcag cttctcgact |
| | tagaccctga agtggaatgt |
| 841 | ctgaaggcca acaccaatga ggttctgtgg gcggtggtgg |
| | cggcgttcac caagtaa |

GOLPH3 protein sequence:

| | |
|---|---|
| 1 | MTSLTQRSSG LVQRRTEASR NAADKERAAG GGAGSSEDDA |
| | QSRRDEQDDD DKGDSKETRL |
| 61 | TLMEEVLLLG LKDREGYTSF WNDCISSGLR GCMLIELALR |
| | GRLQLEACGM RRKSLLTRKV |
| 121 | ICKSDAPTGD VLLDEALKHV KETQPPETVQ NWIELLSGET |
| | WNPLKLHYQL RNVRERLAKN |
| 181 | LVEKGVLTTE KQNFLLFDMT THPLTNNNIK QRLIKKVQEA |
| | VLDKWVNDPH RMDRRLLALI |
| 241 | YLAHASDVLE NAFAPLLDEQ YDLATKRVRQ LLDLDPEVEC |
| | LKANTNEVLW AVVAAFTK |

### II. GOLPH3 Antibodies

An isolated GOLPH3 polypeptide or a fragment thereof (or a nucleic acid encoding such a polypeptide), can be used as an immunogen to generate antibodies that bind to said immunogen, using standard techniques for polyclonal and monoclonal antibody preparation. A full-length GOLPH3 polypeptide can be used, or alternatively, the disclosure relates to antigenic peptide fragments of GOLPH3 polypeptide for use as immunogens. An antigenic peptide of GOLPH3 comprises at least 8 amino acid residues and encompasses an epitope present in the respective full length molecule such that an antibody raised against the peptide forms a specific immune complex with the respective full length molecule. Preferably, the antigenic peptide comprises at least 10 amino acid residues. In one embodiment such epitopes can be specific for a given polypeptide molecule from one species, such as mouse or human (i.e., an antigenic peptide that spans a region of the polypeptide molecule that is not conserved across species is used as immunogen; such non conserved residues can be determined using an alignment such as that provided herein).

In one embodiment, an antibody binds substantially specifically to a GOLPH3 polypeptide, or a fragment thereof. In a preferred embodiment, an antibody binds to a GOLPH3 polypeptide, or a fragment thereof, and blocks the interaction between a GOLPH3 polypeptide or a fragment thereof and its natural binding partner(s) or a fragment(s) thereof.

A GOLPH3 immunogen typically is used to prepare antibodies by immunizing a suitable subject (e.g., rabbit, goat, mouse or other mammal) with the immunogen. An appropriate immunogenic preparation can contain, for example, a recombinantly expressed or chemically synthesized molecule or fragment thereof to which the immune response is to be generated. The preparation can further include an adjuvant, such as Freund's complete or incomplete adjuvant, or similar immunostimulatory agent. Immunization of a suitable subject with an immunogenic preparation induces a polyclonal antibody response to the antigenic peptide contained therein.

Polyclonal antibodies can be prepared as described above by immunizing a suitable subject with a polypeptide immunogen. The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

Any of the many well known protocols used for fusing lymphocytes and immortalized cell lines can be applied for the purpose of generating an anti-GOLPH3 monoclonal antibody (see, e.g., Galfre, G. et al. (1977) Nature 266:55052; Gefter et al. (1977) supra; Lerner (1981) supra; Kenneth (1980) supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a given polypeptide, e.g., using a standard ELISA assay.

As an alternative to preparing monoclonal antibody-secreting hybridomas, a monoclonal antibody specific for one of the above described polypeptides can be identified and isolated by screening a recombinant combinatorial immunoglobulin library (e.g., an antibody phage display library) with the appropriate polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; and McCafferty et al. (1990) Nature 348:552-554.

Additionally, recombinant anti-GOLPH3 polypeptide antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M. European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable generic display package and vectors containing nucleic acid molecules encoding a second polypeptide chain of a single binding pair member using techniques known in the art, e.g., as described in U.S. Pat. No. 5,565,332, 5,871,907, or 5,733,743.

Additionally, fully human antibodies could be made against a GOLPH3 immunogen. Fully human antibodies can be made in mice that are transgenic for human immunoglobulin genes, e.g. according to Hogan, et al., “Manipulating the Mouse Embryo: A Laboratory Manuel,” Cold Spring Harbor Laboratory. Briefly, transgenic mice are immunized with purified GOLPH3 immunogen. Spleen cells are harvested and fused to myeloma cells to produce hybridomas. Hybridomas are selected based on their ability to produce antibodies which bind to the GOLPH3 immunogen. Fully human antibodies would reduce the immunogenicity of such antibodies in a human.

In one embodiment, an antibody for use in the instant invention is a bispecific antibody. A bispecific antibody has binding sites for two different antigens within a single antibody polypeptide. Antigen binding may be simultaneous or sequential. Triomas and hybrid hybridomas are two examples of cell lines that can secrete bispecific antibodies. Examples of bispecific antibodies produced by a hybrid hybridoma or a trioma are disclosed in U.S. Pat. No. 4,474,893. Bispecific antibodies have been constructed by chemical means (Staerz et al. (1985) Nature 314:628, and Perez et al. (1985) Nature 316:354) and hybridoma technology (Staerz and Bevan (1986) Proc. Natl. Acad. Sci. USA, 83:1453, and Staerz and Bevan (1986) Immunol. Today 7:241). Bispecific antibodies are also described in U.S. Pat. No. 5,959,084. Fragments of bispecific antibodies are described in U.S. Pat. No. 5,798,229.

Bispecific agents can also be generated by making heterohybridomas by fusing hybridomas or other cells making different antibodies, followed by identification of clones producing and co-assembling both antibodies. They can also be generated by chemical or genetic conjugation of complete immunoglobulin chains or portions thereof such as Fab and Fv sequences. The antibody component can bind to a GOLPH3 polypeptide or a fragment thereof. In one embodiment, the bispecific antibody could specifically bind to both a GOLPH3 polypeptide or a fragment thereof and its natural binding partner(s) or a fragment(s) thereof.

Yet another aspect of the invention pertains to anti-GOLPH3 antibodies that are obtainable by a process comprising, immunizing an animal with an immunogenic GOLPH3 polypeptide or an immunogenic portion thereof unique to GOLPH3; and then isolating from the animal antibodies that specifically bind to the polypeptide or a fragment thereof.

In another aspect of this invention, GOLPH3 polypeptide fragments or variants can be used. In one embodiment, a variegated library of GOLPH3 variants is generated by combinatorial mutagenesis at the nucleic acid level and is encoded by a variegated gene library. A variegated library of GOLPH3 variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of GOLPH3 (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes GOLPH3. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide.

Alternatively, such peptides can be synthesized by chemical methods. Methods for expression of heterologous proteins in recombinant hosts, chemical synthesis of polypeptides, and in vitro translation are well known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

In one embodiment, the peptide has an amino acid sequence identical or similar to the GOLPH3 binding site of its natural binding partner(s) or a fragment(s) thereof. In one embodiment, the peptide competes with a GOLPH3 polypeptide or a fragment thereof for binding its natural binding partner(s) or a fragment(s) thereof.

Peptides can be produced, typically by direct chemical synthesis, and used e.g., as antagonists of the interactions between a GOLPH3 polypeptide or a fragment thereof and its natural binding partner(s) or a fragment(s) thereof. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, and biochemical properties.

Peptidomimetics (Fauchere, J. (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to peptides useful for diagnostic, prognostic, and/or clinical trial monitoring applications can be used to produce equivalent diagnostic, prognostic, and/or clinical trial monitoring applications. Generally, peptidomimetics are structurally similar to a paradigm polypeptide (i.e., a polypeptide that has a biological or pharmacological activity), such as a human GOLPH3 polypeptide or a fragment thereof, but have one or more peptide linkages optionally replaced by a linkage selected from the group consisting of: —CH2NH—, —CH2S—, —CH2—CH2-, —CH═CH— (cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2—S); Hann, M. M. (1982) J. Chem. Soc. Perkin Trans. I. 307-314 (—CH—CH—, cis and trans); Almquist, R. G. et al. (190) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982)(—CH(OH)CH2-); Holladay, M. W. et al. (1983) Tetrahedron Lett. (1983) 24:4401-4404 (—C(OH)CH2-); and Hruby, V. J. (1982) Life Sci. (1982) 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds. Derivitization (e.g., labeling) of peptidomimetics should not substantially interfere with the desired diagnostic and/or prognostic utility of the peptidomimetic.

These peptides or peptidomimetic molecules can also be chimeric or fusion proteins. As used herein, a “chimeric protein” or “fusion protein” comprises a protein, peptide, or peptidomimetic molecule or a fragment thereof operatively linked to another protein, peptide, or peptidomimetic molecule or a fragment thereof. A “GOLPH3 molecule” refers to a polypeptide having an amino acid sequence corresponding to GOLPH3 or a fragment thereof, whereas a “a non-GOLPH3 molecule” refers to a polypeptide having an amino acid sequence corresponding to a protein which is not substantially homologous to the respective GOLPH3 molecule, e.g., a protein which is different from the GOLPH3 molecule, and which is derived from the same or a different organism. Within a GOLPH3 fusion protein, the GOLPH3 portion can correspond to all or a portion of a full length GOLPH3 molecule. Within the chimeric or fusion protein, the term “operatively linked” is intended to indicate that the independent protein, peptide, or peptidomimetic molecules or fragments thereof are fused in-frame to each other in such a way as to preserve functions exhibited when expressed independently of the fusion.

Such a fusion protein can be produced by recombinant expression of a nucleotide sequence encoding the first peptide and a nucleotide sequence encoding the second peptide.

The second peptide may optionally correspond to a moiety that alters the solubility, affinity, stability or valency of the first peptide, for example, an immunoglobulin constant region. Preferably, the first peptide consists of a portion of GOLPH3 that comprises at least one biologically active portion of a GOLPH3 molecule. In another preferred embodiment, the first peptide consists of a portion of a biologically active molecule. The second peptide can include an immunoglobulin constant region, for example, a human Cγ1 domain or Cγ4 domain (e.g., the hinge, CH2 and CH3 regions of human IgCγ1, or human IgCγ4, see e.g., Capon et al. U.S. Pat. Nos. 5,116,964; 5,580,756; 5,844,095 and the like, incorporated herein by reference). Such constant regions may retain regions which mediate effector function (e.g. Fc receptor binding) or may be altered to reduce effector function. A resulting fusion protein may have altered solubility, binding affinity, stability and/or valency (i.e., the number of binding sites available per polypeptide) as compared to the independently expressed first peptide, and may increase the efficiency of protein purification. Fusion proteins and peptides produced by recombinant techniques can be secreted and isolated from a mixture of cells and medium containing the protein or peptide. Alternatively, the protein or peptide can be retained cytoplasmically and the cells harvested, lysed and the protein isolated. A cell culture typically includes host cells, media and other byproducts. Suitable media for cell culture are well known in the art. Protein and peptides can be isolated from cell culture media, host cells, or both using techniques known in the art for purifying proteins and peptides. Techniques for transfecting host cells and purifying proteins and peptides are known in the art.

Preferably, a fusion protein of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). A polypeptide encoding nucleic acid can be cloned into such an expression vector such that the fusion moiety is linked in-frame to the GOLPH3 encoding sequences.

In another embodiment, the fusion protein contains a heterologous signal sequence at its N-terminus. In certain host cells (e.g., mammalian host cells), expression and/or secretion of a polypeptide can be increased through use of a heterologous signal sequence.

The fusion proteins of the invention can be used as immunogens to produce antibodies in a subject. Such antibodies may be used to purify the respective natural polypeptides from which the fusion proteins were generated, or in screening assays to identify polypeptides which inhibit the interactions between a GOLPH3 polypeptide or a fragment thereof and its natural binding partner(s) or a fragment(s) thereof.

In yet another aspect of the invention, GOLPH3 polypeptides or fragments thereof can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other polypeptides which bind to or interact with GOLPH3 or fragments thereof (“GOLPH3-binding proteins”, “GOLPH3 binding partners”, or “GOLPH3-bp”) and are involved in GOLPH3 activity. Such GOLPH3-binding proteins are also likely to be involved in the propagation of signals by the GOLPH3 polypeptides or GOLPH3 natural binding partner(s) as, for example, downstream elements of a GOLPH3-mediated signaling pathway. Alternatively, such GOLPH3-binding polypeptides may be GOLPH3 inhibitors.

The two-hybrid system is based on the modular nature of most transcription factors, which consist of separable DNA-binding and activation domains. Briefly, the assay utilizes two different DNA constructs. In one construct, the gene that codes for a GOLPH3 polypeptide is fused to a gene encoding the DNA binding domain of a known transcription factor (e.g., GAL-4). In the other construct, a DNA sequence, from a library of DNA sequences, that encodes an unidentified polypeptide (“prey” or “sample”) is fused to a gene that codes for the activation domain of the known transcription factor. If the “bait” and the “prey” polypeptides are able to interact, in vivo, forming a GOLPH3-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the GOLPH3 polypeptide.

### III. Uses and Methods of the Invention

The GOLPH3 molecules, e.g., molecules comprising the GOLPH3 nucleic acid molecules, polypeptides, polypeptide homologues, antibodies, and fragments thereof, described herein can be used in one or more of the following methods: a) screening assays; and b) predictive medicine (e.g., diagnostic assays, prognostic assays, and monitoring clinical trials).

The isolated nucleic acid molecules of the invention can be used, for example, to express a GOLPH3 polypeptide or a fragment thereof and to detect GOLPH3 mRNA or a fragment thereof (e.g., in a biological sample) or a genetic alteration in a GOLPH3 gene, as described further below. Moreover, the anti-GOLPH3 antibodies or fragments thereof of the invention can be used to detect and isolate GOLPH3 polypeptides or fragments thereof.

A. Screening Assays

In one aspect, the invention relates to a method for preventing in a subject, a disease or condition associated with an unwanted or less than desirable immune response. Subjects at risk for a disease that would benefit from treatment with the claimed agents or methods can be identified, for example, by any or a combination of diagnostic or prognostic assays known in the art and described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. This process is called chromosome mapping. Accordingly, portions or fragments of the GOLPH3 nucleotide sequences, described herein, can be used to map the location of the GOLPH3 gene on a chromosome. The mapping of the GOLPH3 sequences to chromosomes is an important first step in correlating these sequences with genes associated with disease.

Briefly, GOLPH3 genes can be mapped to chromosomes by preparing PCR primers (preferably 15-25 by in length) from the GOLPH3 nucleotide sequences. Computer analysis of the GOLPH3 sequences can be used to predict primers that do not span more than one exon in the genomic DNA, thus complicating the amplification process. These primers can then be used for PCR screening of somatic cell hybrids containing individual human chromosomes. Only those hybrids containing the human gene corresponding to the GOLPH3 sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes (D'Eustachio, P. et al. (1983) Science 220:919-924). Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the GOLPH3 nucleotide sequences to design oligonucleotide primers, sublocalization can be achieved with panels of fragments from specific chromosomes. Other mapping strategies which can similarly be used to map a GOLPH3 sequence to its chromosome include in situ hybridization (described in Fan, Y. et al. (1990) Proc. Natl. Acad. Sci. USA 87:6223-27), pre-screening with labeled flow-sorted chromosomes, and pre-selection by hybridization to chromosome specific cDNA libraries.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical such as colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases will suffice to get good results in a reasonable amount of time. For a review of this technique, see Verma et al., Human Chromosomes: A Manual of Basic Techniques (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridization during chromosomal mapping.

Once a sequence has been mapped to a precise chromosomal location, the physical position of the sequence on the chromosome can be correlated with genetic map data (such data are found, for example, in McKusick, V., Mendelian Inheritance in Man, available on-line through Johns Hopkins University Welch Medical Library). The relationship between a gene and a disease, mapped to the same chromosomal region, can then be identified through linkage analysis (co-inheritance of physically adjacent genes), described in, for example, Egeland, J. et al. (1987) Nature 325:783-787.

Moreover, differences in the DNA sequences between individuals affected and unaffected with a disease associated with the GOLPH3 gene can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The GOLPH3 sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the GOLPH3 nucleotide sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The GOLPH3 nucleotide sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of GOLPH3 can provide positive individual identification with a panel of perhaps 10 to 1,000 primers which each yield a noncoding amplified sequence of 100 bases. If predicted GOLPH3 coding sequences are used, a more appropriate number of primers for positive individual identification would be 500-2000.

If a panel of reagents from GOLPH3 nucleotide sequences described herein is used to generate a unique identification database for an individual, those same reagents can later be used to identify tissue from that individual. Using the unique identification database, positive identification of the individual, living or dead, can be made from extremely small tissue samples.

3. Use of GOLPH3 Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of GOLPH3 are particularly appropriate for this use as greater numbers of polymorphisms occur in the noncoding regions, making it easier to differentiate individuals using this technique. Examples of polynucleotide reagents include the GOLPH3 nucleotide sequences or portions thereof, e.g., fragments derived from the noncoding regions of GOLPH3 having a length of at least 20 bases, preferably at least 30 bases.

The GOLPH3 nucleotide sequences described herein can further be used to provide polynucleotide reagents, e.g., labeled or labelable probes which can be used in, for example, an in situ hybridization technique, to identify a specific tissue, e.g., lymphocytes.

This can be very useful in cases where a forensic pathologist is presented with a tissue of unknown origin. Panels of such GOLPH3 probes can be used to identify tissue by species and/or by organ type.

In a similar fashion, these reagents, e.g., GOLPH3 primers or probes can be used to screen tissue culture for contamination (i.e., screen for the presence of a mixture of different types of cells in a culture).

C. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect of the present invention relates to diagnostic assays for determining GOLPH3 polypeptide and/or nucleic acid expression as well as GOLPH3 activity, in the context of a biological sample (e.g., blood, serum, cells, or tissue) to thereby determine whether an individual is afflicted with a disease or disorder, or is at risk of developing a disorder, associated with aberrant or unwanted GOLPH3 expression or activity. The invention also provides for prognostic (or predictive) assays for determining whether an individual is at risk of developing a disorder associated with GOLPH3 polypeptide, nucleic acid expression or activity. For example, mutations in a GOLPH3 gene can be assayed in a biological sample.

Such assays can be used for prognostic or predictive purpose to thereby prophylactically treat an individual prior to the onset of a disorder characterized by or associated with GOLPH3 polypeptide, nucleic acid expression or activity.

Another aspect of the invention pertains to monitoring the influence of agents (e.g., drugs, compounds) on the expression or activity of GOLPH3 in clinical trials. These and other agents are described in further detail in the following sections.

1. Diagnostic Assays

An exemplary method for detecting the presence or absence of GOLPH3 polypeptide or nucleic acid or fragments thereof in a biological sample involves obtaining a biological sample from a test subject and contacting the biological sample with a compound or an agent capable of detecting GOLPH3 polypeptide or nucleic acid that encodes GOLPH3 polypeptide (e.g., mRNA or genomic DNA) or fragments thereof such that the presence of GOLPH3 polypeptide or nucleic acid or fragments thereof is detected in the biological sample. A preferred agent for detecting GOLPH3 mRNA, genomic DNA, or fragments thereof is a labeled nucleic acid probe capable of hybridizing to GOLPH3 mRNA, genomic DNA., or fragments thereof. The nucleic acid probe can be, for example, full length GOLPH3 nucleic acid, or a portion thereof, such as an oligonucleotide of at least 15, 30, 50, 100, 250 or 500 nucleotides in length and sufficient to specifically hybridize under stringent conditions to GOLPH3 mRNA or genomic DNA. Other suitable probes for use in the diagnostic assays of the invention are described herein.

In one embodiment, the level of GOLPH3 protein is measured. It is generally preferred to use antibodies, or antibody equivalents, to detect GOLPH3 protein. Methods for the detection of protein are well known to those skilled in the art, and include ELISA (enzyme linked immunosorbent assay), RIA (radioimmunoassay), Western blotting, and immunohistochemistry. Immunoassays, such as ELISA or RIA, which can be extremely rapid, are more generally preferred. Antibody arrays or protein chips can also be employed, see for example U.S. Patent Application Nos: 20030013208A1; 20020155493A1, 20030017515 and U.S. Pat. Nos. 6,329,209; 6,365,418, herein incorporated by reference in their entirety.

ELISA and RIA procedures may be conducted such that a GOLPH3 standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, GOLPH3 in the sample is allowed to react with the corresponding immobilized antibody, radioisotope- or enzyme-labeled anti-GOLPH3 antibody is allowed to react with the system, and radioactivity or the enzyme assayed (ELISA-sandwich assay). Other conventional methods may also be employed as suitable.

The above techniques may be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods may also be employed as suitable.

In one embodiment, a method for measuring GOLPH3 levels comprises the steps of: contacting a biological specimen with an antibody or variant (e.g., fragment) thereof which selectively binds GOLPH3, and detecting whether said antibody or variant thereof is bound to said sample and thereby measuring the levels of GOLPH3.

Enzymatic and radiolabeling of GOLPH3 and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect GOLPH3 according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Anti-GOLPH3 antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of GOLPH3, e.g., human GOLPH3, e.g., in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

Anti-GOLPH3 antibodies may also be used for imaging purposes, for example, to detect the presence of GOLPH3 in cells and tissues of a subject. Suitable labels include radioisotopes, iodine (¹²⁵I, ¹²¹I) carbon (¹⁴C), sulphur (³⁵S), tritium (³H), indium (¹¹²In), and technetium (⁹⁹ mTc), fluorescent labels, such as fluorescein and rhodamine, and biotin.

For in vivo imaging purposes, antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the patient, such as barium or cesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m. The labeled antibody or antibody fragment will then preferentially accumulate at the location of cells which contain GOLPH3. The labeled antibody or antibody fragment can then be detected using known techniques.

Antibodies that may be used to detect GOLPH3 include any antibody, whether natural or synthetic, full length or a fragment thereof, monoclonal or polyclonal, that binds sufficiently strongly and specifically to the GOLPH3 to be detected, e.g., human GOLPH3. An antibody may have a Kd of at most about 10⁻⁶M, 10⁻⁷M, 10⁻⁸M, 10⁻⁹M, 10⁻¹⁰ M, 10⁻¹¹ M, 10⁻¹²M. The phrase “specifically binds” refers to binding of, for example, an antibody to an epitope or antigen or antigenic determinant in such a manner that binding can be displaced or competed with a second preparation of identical or similar epitope, antigen or antigenic determinant. An antibody may bind preferentially to GOLPH3 relative to other proteins, such as related proteins.

Antibodies are commercially available or may be prepared according to methods known in the art.

Antibodies and derivatives thereof that may be used encompass polyclonal or monoclonal antibodies, chimeric, human, humanized, primatized (CDR-grafted), veneered or single-chain antibodies as well as functional fragments, i.e., GOLPH3 binding fragments, of antibodies. For example, antibody fragments capable of binding to GOLPH3 or portions thereof, including, but not limited to, Fv, Fab, Fab′ and F (ab′)2 fragments can be used. Such fragments can be produced by enzymatic cleavage or by recombinant techniques. For example, papain or pepsin cleavage can generate Fab or F (ab′)2 fragments, respectively. Other proteases with the requisite substrate specificity can also be used to generate Fab or F (ab′)2 fragments. Antibodies can also be produced in a variety of truncated forms using antibody genes in which one or more stop codons have been introduced upstream of the natural stop site. For example, a chimeric gene encoding a F (ab′)2 heavy chain portion can be designed to include DNA sequences encoding the CH, domain and hinge region of the heavy chain.

Synthetic and engineered antibodies are described in, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al. BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies. Antibodies produced from a library, e.g., phage display library, may also be used.

In some embodiments, agents that specifically bind to GOLPH3 other than antibodies are used, such as peptides. Peptides that specifically bind to GOLPH3 can be identified by any means known in the art. For example, specific peptide binders of GOLPH3 can be screened for using peptide phage display libraries.

Generally, agents which are capable of detecting GOLPH3 polypeptide, such that the presence of GOLPH3 is detected and/or quantitated, may be used. As defined herein, an “agent” refers to a substance which is capable of identifying or detecting GOLPH3 in a biological sample (e.g., identifies or detects GOLPH3 mRNA, GOLPH3 DNA, GOLPH3 protein). In one embodiment, the agent is a labeled or labelable antibody which specifically binds to GOLPH3 polypeptide. As used herein, the phrase “labeled or labelable” refers to the attaching or including of a label (e.g., a marker or indicator) or ability to attach or include a label (e.g., a marker or indicator). Markers or indicators include, but are not limited to, for example, radioactive molecules, colorimetric molecules, and enzymatic molecules which produce detectable changes in a substrate.

In addition, the GOLPH3 protein may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference. Mass spectrometry methods are well known in the art and have been used to quantify and/or identify biomolecules, such as proteins (see, e.g., Li et al. (2000) Tibtech 18, 151-160; Rowley et al. (2000) Methods 20, 383-397; Kuster and Mann (1998) Curr. Opin. Structural Biol. 8, 393-400). Further, mass spectrometric techniques have been developed that permit at least partial de novo sequencing of isolated proteins (see, e.g., Chait et al. (1993) Science 262, 89-92; Keough et al. (1999) Proc. Natl. Acad. Sci. USA. 96, 7131-7136; reviewed in Bergman (2000) EXS 88, 133-44).

In certain embodiments, a gas phase ion spectrophotometer is used. In other embodiments, laser-desorption/ionization mass spectrometry is used to analyze the sample. Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes (see, e.g., Hellenkamp et al., U.S. Pat. No. 5,118,937 and Beavis and Chait, U.S. Pat. No. 5,045,694).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied (see, e.g., Hutchens and Yip, U.S. Pat. No. 5,719,060 and Hutchens and Yip, WO 98/59361). The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis) to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to be mixed with the known control sample in the same mass spectrometry run.

In one embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantification of the amount of the biomolecule present in the first sample. In certain embodiments, the identity of the biomolecules in the first and second sample can also be determined.

In one embodiment, detecting or determining GOLPH3 levels comprises detecting or determining GOLPH3 RNA levels. In one embodiment, one or more cells from the subject to be tested are obtained and RNA is isolated from the cells. In a preferred embodiment, a sample of breast tissue cells is obtained from the subject. When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. Tissue samples can be obtained according to methods known in the art.

It is also possible to obtain a cell sample from a subject, and then to enrich it in the desired cell type. For example, cells can be isolated from other cells using a variety of techniques, such as isolation with an antibody binding to an epitope on the cell surface of the desired cell type. Where the desired cells are in a solid tissue, particular cells can be dissected out, e.g., by microdissection.

In one embodiment, RNA is obtained from a single cell. For example, a cell can be isolated from a tissue sample by laser capture microdissection (LCM). Using this technique, a cell can be isolated from a tissue section, including a stained tissue section, thereby assuring that the desired cell is isolated (see, e.g., Bonner et al. (1997) Science 278: 1481; Emmert-Buck et al. (1996) Science 274:998; Fend et al. (1999) Am. J. Path. 154: 61 and Murakami et al. (2000) Kidney Int. 58:1346). For example, Murakami et al., supra, describe isolation of a cell from a previously immunostained tissue section.

It is also be possible to obtain cells from a subject and culture the cells in vitro, such as to obtain a larger population of cells from which RNA can be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the tissue or cells obtained from a subject is snap frozen as soon as possible.

RNA can be extracted from the tissue sample by a variety of methods, e.g., the guanidium thiocyanate lysis followed by CsCl centrifugation (Chirgwin et al., 1979, Biochemistry 18:5294-5299). RNA from single cells can be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample can then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in GOLPH3 sequences. Enrichment can be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, can further be amplified. As defined herein, an “amplification process” is designed to strengthen, increase, or augment a molecule within the RNA. For example, where RNA is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Various amplification and detection methods can be used. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994). Real time PCR may also be used (see Examples).

Other known amplification methods which can be utilized herein include but are not limited to the so-called “NASBA” or “35R” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; target mediated amplification, as described by PCT Publication WO9322461; PCR; ligase chain reaction (LCR) (see, e.g., Wu and Wallace, Genomics 4, 560 (1989), Landegren et al., Science 241, 1077 (1988)); self-sustained sequence replication (SSR) (see, e.g., Guatelli et al., Proc. Nat. Acad. Sci. USA, 87, 1874 (1990)); and transcription amplification (see, e.g., Kwoh et al., Proc. Natl. Acad. Sci. USA 86, 1173 (1989)).

Detection of RNA transcripts may be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylin to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Labeled nucleic acids of a test sample obtained from a patient may be hybridized to a solid surface comprising of GOLPH3 DNA. Positive hybridization signal is obtained with the sample containing GOLPH3 transcripts. Methods of preparing DNA arrays and their use are well known in the art (see, e.g., U.S. Pat. Nos. 6,618,6796; 6,379,897; 6,664,377; 6,451,536; 548,257; U.S. 20030157485 and Schena et al. (1995) Science 20, 467-470; Gerhold et al. (1999) Trends In Biochem. Sci. 24, 168-173; and Lennon et al. (2000) Drug Discovery Today 5, 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescently-labeled cDNA probes are generated. The microarrays capable of hybridizing to GOLPH3 cDNA are then probed with the labeled cDNA probes, the slides scanned and fluorescence intensity measured. This intensity correlates with the hybridization intensity and expression levels.

Types of probes that can be used in the methods described herein include cDNA, riboprobes, synthetic oligonucleotides and genomic probes. The type of probe used will generally be dictated by the particular situation, such as riboprobes for in situ hybridization, and cDNA for Northern blotting, for example. In one embodiment, the probe is directed to nucleotide regions unique to the RNA. The probes may be as short as is required to differentially recognize GOLPH3 mRNA transcripts, and may be as short as, for example, 15 bases; however, probes of at least 17, 18, 19 or 20 or more bases can be used. In one embodiment, the primers and probes hybridize specifically under stringent conditions to a DNA fragment having the nucleotide sequence corresponding to the GOLPH3 gene. As herein used, the term “stringent conditions” means hybridization will occur only if there is at least 95% identity in nucleotide sequences. In another embodiment, hybridization under “stringent conditions” occurs when there is at least 97% identity between the sequences.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

In one embodiment, a change in genomic GOLPH3 copy number (e.g., germline and/or somatic) is detected. In one embodiment, the biological sample is tested for the presence of copy number changes in genomic loci (e.g., germline and/or somatic) containing GOLPH3. A copy number of at least 3, 4, 5, 6, 7, 8, 9, or 10 is indicative of the presence of cancer or the likelihood of developing cancer.

Methods of evaluating the copy number of a particular biomarker or chromosomal region (e.g., GOLPH3 or chromosome 1q32) include, but are not limited to, hybridization-based assays. Hybridization-based assays include, but are not limited to, traditional “direct probe” methods, such as Southern blots, in situ hybridization (e.g., FISH and FISH plus SKY) methods, and “comparative probe” methods, such as comparative genomic hybridization (CGH), e.g., cDNA-based or oligonucleotide-based CGH. The methods can be used in a wide variety of formats including, but not limited to, substrate (e.g. membrane or glass) bound methods or array-based approaches.

In one embodiment, evaluating the copy number of a GOLPH3 gene in a sample involves a Southern Blot. In a Southern Blot, the genomic DNA (typically fragmented and separated on an electrophoretic gel) is hybridized to a probe specific for the target region (e.g., GOLPH3 or chromosome 1q32). Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. Alternatively, a Northern blot may be utilized for evaluating the copy number of encoding nucleic acid in a sample. In a Northern blot, mRNA is hybridized to a probe specific for the target region. Comparison of the intensity of the hybridization signal from the probe for the target region with control probe signal from analysis of normal RNA (e.g., a non-amplified portion of the same or related cell, tissue, organ, etc.) provides an estimate of the relative copy number of the target nucleic acid. Alternatively, other methods well known in the art to detect GOLPH3 RNA can be used, such that higher or lower expression relative to an appropriate control (e.g., a non-amplified portion of the same or related cell tissue, organ, etc.) provides an estimate of the relative copy number of the target nucleic acid.

An alternative means for determining genomic copy number is in situ hybridization (e.g., Angerer (1987) Meth. Enzymol 152: 649). Generally, in situ hybridization comprises the following steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application. In a typical in situ hybridization assay, cells are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained. The probes are typically labeled, e.g., with radioisotopes or fluorescent reporters. In one embodiment, probes are sufficiently long so as to specifically hybridize with the target nucleic acid(s) under stringent conditions. Probes generally range in length from about 200 bases to about 1000 bases. In some applications it is necessary to block the hybridization capacity of repetitive sequences. Thus, in some embodiments, tRNA, human genomic DNA, or Cot-1DNA is used to block non-specific hybridization.

An alternative means for determining genomic copy number is comparative genomic hybridization. In general, genomic DNA is isolated from normal reference cells, as well as from test cells (e.g., tumor cells) and amplified, if necessary. The two nucleic acids are differentially labeled and then hybridized in situ to metaphase chromosomes of a reference cell. The repetitive sequences in both the reference and test DNAs are either removed or their hybridization capacity is reduced by some means, for example by prehybridization with appropriate blocking nucleic acids and/or including such blocking nucleic acid sequences for said repetitive sequences during said hybridization. The bound, labeled DNA sequences are then rendered in a visualizable form, if necessary. Chromosomal regions in the test cells which are at increased or decreased copy number can be identified by detecting regions where the ratio of signal from the two DNAs is altered. For example, those regions that have decreased in copy number in the test cells will show relatively lower signal from the test DNA than the reference compared to other regions of the genome. Regions that have been increased in copy number in the test cells will show relatively higher signal from the test DNA. Where there are chromosomal deletions or multiplications, differences in the ratio of the signals from the two labels will be detected and the ratio will provide a measure of the copy number. In another embodiment of CGH, array CGH (aCGH), the immobilized chromosome element is replaced with a collection of solid support bound target nucleic acids on an array, allowing for a large or complete percentage of the genome to be represented in the collection of solid support bound targets. Target nucleic acids may comprise cDNAs, genomic DNAs, oligonucleotides (e.g., to detect single nucleotide polymorphisms) and the like. Array-based CGH may also be performed with single-color labeling (as opposed to labeling the control and the possible tumor sample with two different dyes and mixing them prior to hybridization, which will yield a ratio due to competitive hybridization of probes on the arrays). In single color CGH, the control is labeled and hybridized to one array and absolute signals are read, and the possible tumor sample is labeled and hybridized to a second array (with identical content) and absolute signals are read. Copy number difference is calculated based on absolute signals from the two arrays. Methods of preparing immobilized chromosomes or arrays and performing comparative genomic hybridization are well known in the art (see, e.g., U.S. Pat. Nos. 6,335,167; 6,197,501; 5,830,645; and 5,665,549 and Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), etc.) In another embodiment, the hybridization protocol of Pinkel, et al. (1998) Nature Genetics 20: 207-211, or of Kallioniemi (1992) Proc. Natl. Acad Sci USA 89:5321-5325 (1992) is used. In still another embodiment, amplification-based assays can be used to measure copy number. In such amplification-based assays, the nucleic acid sequences act as a template in an amplification reaction (e.g., Polymerase Chain Reaction (PCR). In a quantitative amplification, the amount of amplification product will be proportional to the amount of template in the original sample. Comparison to appropriate controls, e.g. healthy tissue, provides a measure of the copy number.

Methods of “quantitative” amplification are well known to those of skill in the art.

For example, quantitative PCR involves simultaneously co-amplifying a known quantity of a control sequence using the same primers. This provides an internal standard that may be used to calibrate the PCR reaction. Detailed protocols for quantitative PCR are provided in Innis, et al. (1990) PCR Protocols, A Guide to Methods and Applications, Academic Press, Inc. N.Y.). Measurement of DNA copy number at microsatellite loci using quantitative PCR analysis is described in Ginzonger, et al. (2000) Cancer Research 60:5405-5409. The known nucleic acid sequence for the genes is sufficient to enable one of skill in the art to routinely select primers to amplify any portion of the gene. Fluorogenic quantitative PCR may also be used in the methods of the invention. In fluorogenic quantitative PCR, quantitation is based on amount of fluorescence signals, e.g., TaqMan and SYBR green.

Other suitable amplification methods include, but are not limited to, ligase chain reaction (LCR) (see Wu and Wallace (1989) Genomics 4: 560, Landegren, et al. (1988) Science 241:1077, and Barringer et al. (1990) Gene 89: 117), transcription amplification (Kwoh, et al. (1989) Proc. Natl. Acad. Sci. USA 86: 1173), self-sustained sequence replication (Guatelli, et al. (1990) Proc. Nat. Acad. Sci. USA 87: 1874), dot PCR, and linker adapter PCR, etc.

Loss of heterozygosity (LOH) mapping (Wang, Z. C., et al. (2004) Cancer Res 64(1):64-71; Seymour, A. B., et al. (1994) Cancer Res 54, 2761-4; Hahn, S. A., et al. (1995) Cancer Res 55, 4670-5; Kimura, M., et al. (1996) Genes Chromosomes Cancer 17, 88-93) may also be used to identify regions of amplification or deletion.

In one embodiment, the biological sample contains polypeptide molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting GOLPH3 polypeptide, mRNA, genomic DNA, or fragments thereof, such that the presence of GOLPH3 polypeptide, mRNA, genomic DNA, or fragments thereof, is detected in the biological sample, and comparing the presence of GOLPH3 polypeptide, mRNA, genomic DNA, or fragments thereof, in the control sample with the presence of GOLPH3 polypeptide, mRNA, genomic DNA, or fragments thereof in the test sample.

The invention also encompasses kits for detecting the presence of a GOLPH3 nucleic acid, polypeptide, or fragments thereof, in a biological sample. For example, the kit can comprise a labeled compound or agent capable of detecting a GOLPH3 nucleic acid, polypeptide, or fragments thereof in a biological sample; means for determining the amount of the GOLPH3 nucleic acid, polypeptide, or fragments thereof in the sample; and means for comparing the amount of the GOLPH3 nucleic acid, polypeptide, or fragments thereof in the sample with a standard. The compound or agent can be packaged in a suitable container. The kit can further comprise instructions for using the kit to detect the GOLPH3 nucleic acid, polypeptide, or fragments thereof.

2. Prognostic Assays

The diagnostic methods described herein can furthermore be utilized to identify subjects having or at risk of developing a disease or disorder associated with aberrant or unwanted GOLPH3 expression or activity. As used herein, the term “aberrant” includes a GOLPH3 expression or activity which deviates from the wild type GOLPH3 expression or activity. Aberrant expression or activity includes increased or decreased expression or activity, as well as expression or activity which does not follow the wild type developmental pattern of expression or the subcellular pattern of expression. For example, aberrant GOLPH3 expression or activity is intended to include the cases in which a mutation in the GOLPH3 gene or regulatory sequence thereof causes the GOLPH3 gene to be under-expressed or over-expressed and situations in which such mutations result in a non-functional GOLPH3 polypeptide or a polypeptide which does not function in a wild-type fashion, e.g., a polypeptide which does not interact with a GOLPH3 binding partner(s) or one which interacts with a non-GOLPH3 binding partner(s). As used herein, the term “unwanted” includes an unwanted phenomenon involved in a biological response such as immune cell activation. For example, the term unwanted includes a GOLPH3 expression or activity which is undesirable in a subject.

The assays described herein, such as the preceding diagnostic assays or the following assays, can be utilized to identify a subject having or at risk of developing a disorder associated with a misregulation in GOLPH3 polypeptide activity or nucleic acid expression, such as a cancer, e.g., lung, ovarian, pancreatic, liver, breast, prostate, and colon carcinomas, as well as melanoma and multiple myeloma. Alternatively, the prognostic assays can be utilized to identify a subject having or at risk for developing a disorder associated with a misregulation of GOLPH3 polypeptide activity or nucleic acid expression, such as a cancer, e.g., lung, ovarian, pancreatic, liver, breast, prostate, and colon carcinomas, as well as melanoma and multiple myeloma. Thus, the present invention provides a method for identifying a disease or disorder associated with aberrant or unwanted GOLPH3 expression or activity in which a test sample is obtained from a subject and GOLPH3 polypeptide or nucleic acid (e.g., mRNA or genomic DNA) is detected, wherein the presence of GOLPH3 polypeptide or nucleic acid is diagnostic for a subject having or at risk of developing a disease or disorder associated with aberrant or unwanted GOLPH3 expression or activity. As used herein, a “test sample” refers to a biological sample obtained from a subject of interest. For example, a test sample can be a biological fluid (e.g., cerebrospinal fluid or serum), cell sample, or tissue.

Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat a disease or disorder associated with aberrant or unwanted GOLPH3 expression or activity. For example, such methods can be used to determine whether a subject can be effectively treated with an agent for a cancer, e.g., lung, ovarian, pancreatic, liver, breast, prostate, and colon carcinomas, as well as melanoma and multiple myeloma. Thus, the present invention provides methods for determining whether a subject can be effectively treated with an agent for a disorder associated with aberrant or unwanted GOLPH3 expression or activity in which a test sample is obtained and GOLPH3 polypeptide or nucleic acid expression or activity is detected (e.g., wherein the abundance of GOLPH3 polypeptide or nucleic acid expression or activity is diagnostic for a subject that can be administered the agent to treat a disorder associated with aberrant or unwanted GOLPH3 expression or activity).

The methods of the invention can also be used to detect genetic alterations in a GOLPH3 gene, thereby determining if a subject with the altered gene is at risk for a disorder characterized by misregulation in GOLPH3 polypeptide activity or nucleic acid expression, such as cancer, e.g., lung, ovarian, pancreatic, liver, breast, prostate, and colon carcinomas, as well as melanoma and multiple myeloma. In preferred embodiments, the methods include detecting, in a sample of cells from the subject, the presence or absence of a genetic alteration characterized by at least one alteration affecting the integrity of a gene encoding a GOLPH3 polypeptide, or the mis-expression of the GOLPH3 gene. For example, such genetic alterations can be detected by ascertaining the existence of at least one of 1) a deletion of one or more nucleotides from a GOLPH3 gene, 2) an addition of one or more nucleotides to a GOLPH3 gene, 3) a substitution of one or more nucleotides of a GOLPH3 gene, 4) a chromosomal rearrangement of a GOLPH3 gene, 5) an alteration in the level of a messenger RNA transcript of a GOLPH3 gene, 6) aberrant modification of a GOLPH3 gene, such as of the methylation pattern of the genomic DNA, 7) the presence of a non-wild type splicing pattern of a messenger RNA transcript of a GOLPH3 gene, 8) a non-wild type level of a GOLPH3 polypeptide, 9) allelic loss or gain of a GOLPH3 gene, and 10) inappropriate post-translational modification of a GOLPH3 polypeptide. As described herein, there are a large number of assays known in the art which can be used for detecting alterations in a GOLPH3 gene. A preferred biological sample is a tissue or serum sample isolated by conventional means from a subject.

In certain embodiments, detection of the alteration involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a GOLPH3 gene (see Abravaya et al. (1995) Nucleic Acids Res. 23:675-682). This method can include the steps of collecting a sample of cells from a subject, isolating nucleic acid (e.g., genomic, mRNA or both) from the cells of the sample, contacting the nucleic acid sample with one or more primers which specifically hybridize to a GOLPH3 gene under conditions such that hybridization and amplification of the GOLPH3 gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al. (1990) Proc. Natl. Acad. Sci. USA 87:1874-1878), transcriptional amplification system (Kwoh, D. Y. et al. (1989) Proc. Natl. Acad. Sci. USA 86:1173-1177), Q-Beta Replicase (Lizardi, P. M. et al. (1988) Bio-Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a GOLPH3 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In other embodiments, genetic mutations in GOLPH3 can be identified by hybridizing a sample and control nucleic acids, e.g., DNA or RNA, to high density arrays containing hundreds or thousands of oligonucleotide probes (Cronin, M. T. et al. (1996) Hum. Mutat. 7:244-255; Kozal, M. J. et al. (1996) Nat. Med. 2:753-759). For example, genetic mutations in GOLPH3 can be identified in two dimensional arrays containing light-generated DNA probes as described in Cronin et al. (1996) supra. Briefly, a first hybridization array of probes can be used to scan through long stretches of DNA in a sample and control to identify base changes between the sequences by making linear arrays of sequential, overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene. Such genetic mutations in GOLPH3 can be identified in a variety of contexts, including, for example, germline and somatic mutations.

In yet another embodiment, any of a variety of sequencing reactions known in the art can be used to directly sequence the GOLPH3 gene and detect mutations by comparing the sequence of the sample GOLPH3 with the corresponding wild-type (control) sequence. Examples of sequencing reactions include those based on techniques developed by Maxam and Gilbert (1977) Proc. Natl. Acad. Sci. USA 74:560 or Sanger (1977) Proc. Natl. Acad. Sci. USA 74:5463. It is also contemplated that any of a variety of automated sequencing procedures can be utilized when performing the diagnostic assays (Naeve, C. W. (1995) Biotechniques 19:448-53), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the GOLPH3 gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes formed by hybridizing (labeled) RNA or DNA containing the wild-type GOLPH3 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al. (1988) Proc. Natl. Acad. Sci. USA 85:4397 and Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GOLPH3 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a GOLPH3 sequence, e.g., a wild-type GOLPH3 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in GOLPH3 genes. For example, single strand conformation polymorphism (SSCP) may be used to detect differences in electrophoretic mobility between mutant and wild type nucleic acids (Orita et al. (1989) Proc Natl. Acad. Sci. USA 86:2766; see also Cotton (1993) Mutat. Res. 285:125-144 and Hayashi (1992) Genet. Anal. Tech. Appl. 9:73-79). Single-stranded DNA fragments of sample and control GOLPH3 nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to ensure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753). Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163; Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection (Gasparini et al. (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a GOLPH3 gene.

Furthermore, any cell type or tissue in which GOLPH3 is expressed may be utilized in the prognostic assays described herein.

In another embodiment, a method is provided to assess the likelihood of efficacy of an mTOR pathway inhibitor in a subject. GOLPH3 has been found herein to confer increased sensitivity to rapamycin, which is an inhibitor of the mTOR pathway, such that GOLPH3 expression level or DNA copy number status comprises a positive predictive biomarker for mTOR pathway inhibitors (e.g., rapamycin, CCl-779, everolimus, CC-5013, AP23573, TAFA93, deforolimus, etc.) in cancers, including, e.g., lung, ovarian, pancreatic, liver, breast, prostate, and colon carcinomas, as well as melanoma and multiple myeloma. Without being bound by theory, GOLPH3 over-expressing cells display the well-described phenomenon of “oncogene addiction,” wherein cells become “addicted” to an oncogene (e.g., GOLPH3) and inhibiting the targeted pathway (e.g., the mTOR pathway) attenuates growth of the cells and/or tumor in question.

3. Monitoring of Effects During Clinical Trials

Monitoring the influence of agents (e.g., drugs) on the expression or activity of a GOLPH3 polypeptide or a fragment thereof (e.g., the modulation of cell proliferation and/or migration) can be applied not only in basic drug screening, but also in clinical trials. For example, the effectiveness of an agent determined by a screening assay as described herein to increase GOLPH3 gene expression, polypeptide levels, or upregulate GOLPH3 activity, can be monitored in clinical trials of subjects exhibiting decreased GOLPH3 gene expression, polypeptide levels, or downregulated GOLPH3 activity. Alternatively, the effectiveness of an agent determined by a screening assay to decrease GOLPH3 gene expression, polypeptide levels, or downregulate GOLPH3 activity, can be monitored in clinical trials of subjects exhibiting increased GOLPH3 gene expression, polypeptide levels, or GOLPH3 activity. In such clinical trials, the expression or activity of a GOLPH3 gene, and preferably, other genes that have been implicated in, for example, a GOLPH3-associated disorder can be used as a “read out” or marker of the phenotype of a particular cell.

For example, and not by way of limitation, genes, including GOLPH3, that are modulated in cells by treatment with an agent (e.g., compound, drug or small molecule) which modulates GOLPH3 activity (e.g., identified in a screening assay as described herein) can be identified. Thus, to study the effect of agents on GOLPH3-associated disorders (e.g., disorders characterized by dysregulated GOLPH3 activity), for example, in a clinical trial, cells can be isolated and RNA prepared and analyzed for the levels of expression of GOLPH3 and other genes implicated in the GOLPH3-associated disorder, respectively. The levels of gene expression (e.g., a gene expression pattern) can be quantified by Northern blot analysis or RT-PCR, as described herein, or alternatively by measuring the amount of polypeptide produced, by one of the methods as described herein, or by measuring the levels of activity of GOLPH3 or other genes. In this way, the gene expression pattern can serve as a marker, indicative of the physiological response of the cells to the agent. Accordingly, this response state may be determined before, and at various points during treatment of the individual with the agent.

In a preferred embodiment, the present invention provides a method for monitoring the effectiveness of treatment of a subject with an agent (e.g., an agonist, antagonist, peptidomimetic, polypeptide, peptide, nucleic acid, small molecule, or other drug candidate identified by the screening assays described herein acting directly or indirectly on GOLPH3) including the steps of (i) obtaining a pre-administration sample from a subject prior to administration of the agent; (ii) detecting the level of expression of a GOLPH3 polypeptide, mRNA, genomic DNA, or fragments thereof in the preadministration sample; (iii) obtaining one or more post-administration samples from the subject; (iv) detecting the level of expression or activity of the GOLPH3 polypeptide, mRNA, genomic DNA, or fragments thereof in the post-administration samples; (v) comparing the level of expression or activity of the GOLPH3 polypeptide, mRNA, genomic DNA, or fragments thereof in the pre-administration sample with the GOLPH3 polypeptide, mRNA, or genomic DNA in the post administration sample or samples; and (vi) altering the administration of the agent to the subject accordingly. For example, increased administration of the agent may be desirable to increase the expression or activity of GOLPH3 directly or indirectly to higher levels than detected, i.e., to increase the effectiveness of the agent. Alternatively, decreased administration of the agent may be desirable to decrease expression or activity of GOLPH3 directly or indirectly to lower levels than detected, i.e., to decrease the effectiveness of the agent. According to such an embodiment, GOLPH3 expression or activity may be used as an indicator of the effectiveness of an agent, even in the absence of an observable phenotypic response.

D. GOLPH3-Based Therapeutics for Treating Cancers

Based at least on the observation that high GOLPH3 levels in primary tumors is associated with the presence of cancer, it may be possible to diminish the likelihood of developing cancer, halt the progression of cancer or prevent cancer altogether by inhibiting or reducing the expression level of GOLPH3 in the tumor or tissue of the subject. In one embodiment, a method for treating or preventing cancer, such as lung, ovarian, pancreatic, liver, breast, prostate, and colon carcinomas, as well as melanoma and multiple myeloma, comprises reducing the level of expression of GOLPH3. A method may include reducing the expression of a GOLPH3 gene, reducing the amount of GOLPH3 protein, or inhibiting the activity of a GOLPH3 protein. In a method for treatment, one may reduce GOLPH3 levels or activity in a tumor, e.g., a primary tumor. In a method for preventing cancer, one may reduce GOLPH3 levels or activity in tissue likely to develop cancer, e.g., tissue that exhibits high levels of GOLPH3 expression.

Prophylaxis may be appropriate even at very early stages of the disease, to prevent tumorigenesis or metastasis. Thus, administration of an agent that reduces GOLPH3 levels or activity may be effected as soon as cancer is diagnosed, and treatment continued for as long as is necessary, generally until the threat of the disease has been removed. Such treatment may also be used prophylactically in individuals at high risk for development of certain cancers, e.g., breast cancer.

1. RNAi Technology

In one embodiment, GOLPH3 levels are decreased by administration of or expression in a subject, e.g., in cells or a tissue of the subject, of one or more siRNAs.

Isolated RNA molecules specific to GOLPH3 mRNA, which mediate RNAi, are antagonists useful in the method of the present invention (see, e.g., U.S. Patent Application Nos: 20030153519A1; 20030167490A1; and U.S. Pat. Nos. 6,506,559; 6,573,099, which are herein incorporated by reference in their entirety).

In one embodiment, the RNA is comprised of, or capable of being cleaved to, short interfering or small interfering RNAs (siRNAs). The term “short interfering RNAs (siRNA)” as used herein is intended to refer to any nucleic acid molecule capable of mediating RNAi or gene silencing. The term siRNA is intended to encompass various naturally generated or synthetic compounds, with RNAi function. Such compounds include, without limitation, duplex synthetic oligonucleotides, of about 21 to 23 base pairs with terminal overlaps of 2 or 3 base pairs; hairpin structures of one oligonucleotide chain with sense and complementary, hybridizing, segments of 21-23 base pairs joined by a loop of 3-5 base pairs; and various genetic constructs leading to the expression of the preceding structures or functional equivalents. Such genetic constructs are usually prepared in vitro and introduced in the test system, but can also include siRNA from naturally occurring siRNA precursors coded by the genome of the host cell or animal.

It is not a requirement that the siRNA be comprised solely of RNA. In one embodiment, the siRNA comprises one or more chemical modifications and/or nucleotide analogues. The modification and/or analogue may be any modification and/or analogue, respectively, that does not negatively affect the ability of the siRNA to inhibit GOLPH3 expression. The inclusion of one or more chemical modifications and/or nucleotide analogues in an siRNA may be used to prevent or slow nuclease digestion, and in turn, create a more stable siRNA for practical use. Chemical modifications and/or nucleotide analogues which stabilize RNA are known in the art. Phosphorothioate derivatives, which include the replacement of non-bridging phosphoryl oxygen atoms with sulfur atoms, are one example of analogues showing increased resistance to nuclease digestion. Sites of the siRNA which may be targeted for chemical modification include the loop region of a hairpin structure, the 5′ and 3′ ends of a hairpin structure (e.g. cap structures), the 3′ overhang regions of a double-stranded linear siRNA, the 5′ or 3′ ends of the sense strand and/or antisense strand of a linear siRNA, and one or more nucleotides of the sense and/or antisense strand.

As used herein, the term siRNA is intended to be equivalent to any term in the art defined as a molecule capable of mediating sequence-specific RNAi. Such equivalents include, for example, double-stranded RNA (dsRNA), microRNA (mRNA), short hairpin RNA (shRNA), short interfering oligonucleotide, and post-transcriptional gene silencing RNA (ptgsRNA).

siRNAs may be introduced into cells to suppress gene expression for therapeutic or prophylactic purposes as described in International Publication Number WO 0175164. Such molecules may be introduced into cells to suppress gene expression for therapeutic or prophylactic purposes as described in various patents, patent applications and papers. Publications herein incorporated by reference, describing RNAi technology include, but are not limited to, the following: U.S. Pat. No. 6,686,463, U.S. Pat. No. 6,673,611, U.S. Pat. No. 6,623,962, U.S. Pat. No. 6,506,559, U.S. Pat. No. 6,573,099, and U.S. Pat. No. 6,531,644; International Publication Numbers WO04061081; WO04052093; WO04048596; WO04048594; WO04048581; WO04048566; WO04046320; WO04044537; WO04043406; WO04033620; WO04030660; WO04028471; WO 0175164. Papers which describe the methods and concepts for the optimal use of these compounds include, but are not limited to, the following: Brummelkamp Science 296: 550-553 (2002); Caplen Expert Opin. Biol. Ther. 3:575-86 (2003); Brummelkamp, Science Express 21 Mar. 3 1-6 (2003); Yu Proc Natl Acad Sci USA 99:6047-52 (2002); Paul, Nature Biotechnology 29:505-8 (2002); Paddison, Proc Natl Acad Sci USA 99:1443-8 (2002); Brummelkamp, Nature 424: 797-801 (2003); Brummelkamp, Science 296: -550-3 (2003); Sui, Proc Natl Acad Sci USA 99: 5515-20 (2002); Paddison, Genes and Development 16:948-58 (2002).

A composition comprising an siRNA effective to inhibit GOLPH3 expression may include an RNA duplex comprising a sense sequence of GOLPH3. In this embodiment, the RNA duplex comprises a first strand comprising a sense sequence of GOLPH3 and a second strand comprising a reverse complement of the sense sequence of GOLPH3. In one embodiment the sense sequence of GOLPH3 comprises of from 10 to 25 nucleotides in length. In another embodiment, the sense sequence of GOLPH3 comprises of from 19 to 25 nucleotides in length. In yet another embodiment, the sense sequence of GOLPH3 comprises of from 21 to 23 nucleotides in length. The sense sequence of GOLPH3 can comprises a sequence of GOLPH3 containing a translational start site or a portion of GOLPH3 sequence within the first 400 nucleotides of the human GOLPH3 mRNA.

In another embodiment, a composition comprising an siRNA effective to inhibit GOLPH3 expression may comprise in a single molecule a sense sequence of GOLPH3, the reverse complement of the sense sequence of GOLPH3, and an intervening sequence enabling duplex formation between the sense and reverse complement sequences. The sense sequence of GOLPH3 may comprise 10 to 25 nucleotides in length, 19 to 25 nucleotides in length, or 21 to 23 nucleotides in length.

It will be readily apparent to one of skill in the art that an siRNA of the present invention may comprise a sense sequence of GOLPH3 or the reverse complement of the sense sequence of GOLPH3 which is less than perfectly complementary to each other or to the targeted region of GOLPH3. In other words, the siRNA may comprise mismatches or bulges within the sense or reverse complement sequence. In one aspect, the sense sequence or its reverse complement may not be entirely contiguous. The sequence or sequences may comprise one or more substitutions, deletions, and/or insertions. The only requirement of the present invention is that the siRNA sense sequence possess enough complementarity to its reverse complement and to the targeted region of GOLPH3 to allow for RNAi activity. It is an object of the present invention, therefore, to provide for sequence modifications of an siRNA of the present invention that retain sufficient complementarity to allow for RNAi activity. One of skill in the art may predict that a modified siRNA composition of the present invention will work based on the calculated binding free energy of the modified sequence for the complement sequence and targeted region of GOLPH3. Methods for calculating binding free energies for nucleic acids and the effect of such values on strand hybridization are known in the art.

A wide variety of delivery systems are available for use in delivering an siRNA of the present invention to a target cell in vitro and in vivo. An siRNA of the present invention may be introduced directly or indirectly into a cell in which GOLPH3 inhibition is desired. An siRNA may be directly introduced into a cell by, for example, injection. As such, it is an object of the invention to provide for a composition comprising an siRNA effective to inhibit GOLPH3 in injectable, dosage unit form. An siRNA of the present invention may be injected intravenously or subcutaneously, as an example, for therapeutic use in conjunction with the methods and compositions of the present invention. Such treatment may include intermittent or continuous administration until therapeutically effective levels are achieved to inhibit GOLPH3 expression in the desired tissue.

Indirectly, an expressible DNA sequence or sequences encoding the siRNA may be introduced into a cell and the siRNA, thereafter, transcribed from the DNA sequence or sequences. It is an object of the present invention, therefore, to provide for compositions comprising a DNA sequence or sequences which encode an siRNA effective to inhibit GOLPH3 expression.

A DNA composition of the present invention comprises a first DNA sequence which encodes a first RNA sequence comprising a sense sequence of GOLPH3 and a second DNA sequence which encodes a second RNA sequence comprising the reverse complement of the sense sequence of GOLPH3. The first and second RNA sequences, when hybridized, form an siRNA duplex capable of forming an RNA-induced silencing complex, the RNA-induced silencing complex being capable of inhibiting GOLPH3 expression. The first and second DNA sequences may be chemically synthesized or synthesized by PCR using appropriate primers to GOLPH3. Alternatively, the DNA sequences may be obtained by recombinant manipulation using cloning technology, which is well known in the art. Once obtained, the DNA sequences may be purified, combined, and then introduced into a cell in which GOLPH3 inhibition is desired. Alternatively, the sequences may be contained in a single vector or separate vectors and the vector or vectors introduced into the cell in which GOLPH3 inhibition is desired.

Delivery systems available for use in delivering a DNA composition of the present invention to a target cell include, for example, viral and non-viral systems. Examples of suitable viral systems include, for example, adenoviral vectors, adeno-associated virus, lentivirus, poxvirus, retroviral vectors, vaccinia, herpes simplex virus, HIV, the minute virus of mice, hepatitis B virus and influenza virus. Non-viral delivery systems may also be used, for example using, uncomplexed DNA, DNA-liposome complexes, DNA-protein complexes and DNA-coated gold particles, bacterial vectors such as salmonella, and other technologies such as those involving VP22 transport protein, Co-X-gene, and replicon vectors. A viral or non-viral vector in the context of the present invention may express the antigen of interest.

2. Antisense Technology

In another embodiment, the level of GOLPH3 is reduced or decreased by administration or the expression of antisense molecules in a subject or tissue or cell thereof.

Gene expression can be controlled through triple-helix formation or antisense DNA or RNA, both of which methods are based on binding of a polynucleotide to DNA or RNA. An antisense nucleic acid molecule which is complementary to a nucleic acid molecule encoding GOLPH3 can be designed based upon the isolated nucleic acid molecules encoding GOLPH3. An antisense nucleic acid molecule can comprise a nucleotide sequence which is complementary to a coding strand of a nucleic acid, e.g. complementary to an mRNA sequence, constructed according to the rules of Watson and Crick base pairing, and can hydrogen bond to the coding strand of the nucleic acid. The antisense sequence complementary to a sequence of an mRNA can be complementary to a sequence in the coding region of the mRNA or can be complementary to a 5′ or 3′ untranslated region of the mRNA. Furthermore, an antisense nucleic acid can be complementary in sequence to a regulatory region of the gene encoding the mRNA, for instance, a transcription initiation sequence or regulatory element. In one embodiment, an antisense nucleic acid complementary to a region preceding or spanning the initiation codon or in the 3′ untranslated region of an mRNA is used. An antisense nucleic acid can be designed based upon the nucleotide sequence of GOLPH3. A nucleic acid is designed which has a sequence complementary to a sequence of the coding or untranslated region of the shown nucleic acid. Alternatively, an antisense nucleic acid can be designed based upon sequences of the GOLPH3 gene, which can be identified by screening a genomic DNA library with an isolated nucleic acid of the invention. For example, the sequence of an important regulatory element can be determined by standard techniques and a sequence which is antisense to the regulatory element can be designed.

The antisense nucleic acids and oligonucleotides of the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids. For example, phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub, H. et al., Antisense RNA as a molecular tool for genetic analysis, Reviews—Trends in Genetics, Vol. 1 (1)1986.

In addition, ribozymes can be used to inhibit in vitro expression of GOLPH3. For example, the nucleic acids of the invention can further be used to design ribozymes which are capable of cleaving a single-stranded nucleic acid encoding a GOLPH3 protein, such as a GOLPH3 mRNA transcript. A catalytic RNA (ribozyme) having ribonuclease activity can be designed which has specificity for an mRNA encoding GOLPH3 based upon the sequence of a nucleic acid of the invention. For example, a derivative of a Tetrahymena L-19 IVS RNA can be constructed in which the base sequence of the active site is complementary to the base sequence to be cleaved in a GOLPH3-encoding mRNA (see, e.g., Cech et al., U.S. Pat. No. 4,987,071; Cech, et al., U.S. Pat. No. 5,116,742). Alternatively, a nucleic acid of the invention could be used to select a catalytic RNA having a specific ribonuclease activity from a pool of RNA molecules (see, e.g., Bartel and Szostak (1993) Science 261, 1411-1418). RNA-mediated interference (RNAi) (Fire et al. (1998) Nature 391, 806-811) may also be used.

3. GOLPH3 Blocking Antibodies or Aptamers

In yet another embodiment, GOLPH3 levels are reduced by administration to or expression in a subject or a cell or tissue thereof, of GOLPH3 blocking antibodies or aptamers.

Antibodies, or their equivalents and derivatives, e.g., intrabodies, or other GOLPH3 antagonists, may be used in accordance with the present invention for the treatment or prophylaxis of cancers. Administration of a suitable dose of the antibody or the antagonist may serve to block the activity of the protein and this may provide a crucial time window in which to treat malignant growth.

A method of treatment involves attachment of a suitable toxin to the antibodies which then target the area of the tumor. Such toxins are well known in the art, and may comprise toxic radioisotopes, heavy metals, enzymes and complement activators, as well as such natural toxins as ricin which are capable of acting at the level of only one or two molecules per cell. It may also be possible to use such a technique to deliver localized doses of suitable physiologically active compounds, which may be used, for example, to treat cancers.

In addition to using antibodies to inhibit GOLPH3, it may also be possible to use other forms of inhibitors. For example, it may be possible to identify antagonists that functionally inhibit GOLPH3. In addition, it may also be possible to interfere with the binding of GOLPH3 to target proteins. Other suitable inhibitors will be apparent to the skilled person.

The antibody (or other inhibitors or intrabody) can be administered by a number of methods. One method is set forth by Marasco and Haseltine in PCT WO94/02610, which is incorporated herein by reference. This method discloses the intracellular delivery of a gene encoding the antibody. In one embodiment, a gene encoding a single chain antibody is used. In another embodiment, the antibody would contain a nuclear localization sequence (e.g. an SV40 nuclear localization signal). By this method, one can intracellularly express an antibody, which can block GOLPH3 functioning in desired cells.

Where the present invention provides for the administration of, for example, antibodies to a patient, then this may be by any suitable route. If the tumor is still thought to be, or diagnosed as, localized, then an appropriate method of administration may be by injection direct to the site. Administration may also be by injection, including subcutaneous, intramuscular, intravenous and intradermal injections.

Aptamers can be produced using the methodology disclosed in a U.S. Pat. No. 5,270,163 and WO 91/19813.

4. Other GOLPH3 Inhibitors

Compounds that inhibit the activity of GOLPH3 may also be used. Such compounds include small molecules, e.g., molecules that interact with the active site or a binding site of the protein, e.g., an RNA binding site. Such compounds may be identified according to methods known in the art.

E. Pharmaceutical Formulations

Formulations may be any that are appropriate to the route of administration, and will be apparent to those skilled in the art. The formulations may contain a suitable carrier, such as saline, and may also comprise bulking agents, other medicinal preparations, adjuvants and any other suitable pharmaceutical ingredients. Catheters constitute another mode of administration.

The term “pharmaceutically acceptable” refers to compounds and compositions which may be administered to mammals without undue toxicity. Exemplary pharmaceutically acceptable salts include mineral acid salts such as hydrochlorides, hydrobromides, phosphates, sulfates, and the like; and the salts of organic acids such as acetates, propionates, malonates, benzoates, and the like.

The antibodies, nucleic acids or antagonists of the invention may be administered orally, topically, or by parenteral means, including subcutaneous and intramuscular injection, implantation of sustained release depots, intravenous injection, intranasal administration, and the like. Accordingly, antibodies or nucleic acids of the invention may be administered as a pharmaceutical composition comprising the antibody or nucleic acid of the invention in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol®, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene® (Marion), Aquaphor® (Duke Laboratories), and the like. Other topical formulations include aerosols, bandages, and other wound dressings. Alternatively, one may incorporate or encapsulate the compounds in a suitable polymer matrix or membrane, thus providing a sustained-release delivery device suitable for implantation near the site to be treated locally. Other devices include indwelling catheters and devices such as the Alzet® minipump. Ophthalmic preparations may be formulated using commercially available vehicles such as Sorbi-care® (Allergan), Neodecadron® (Merck, Sharp & Dohme), Lacrilube®, and the like, or may employ topical preparations such as that described in U.S. Pat. No. 5,124,155, incorporated herein by reference. Further, one may provide an antagonist in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of antibody, nucleic acid or inhibitor required to treat any particular disorder will of course vary depending upon the nature and severity of the disorder, the age and condition of the subject, and other factors readily determined by one of ordinary skill in the art.

1. Immunotherapy

In further aspects, the present invention provides methods for using GOLPH3 or an immunoreactive polypeptide thereof (or DNA encoding the protein or polypeptides) for immunotherapy of cancer in a patient. As used herein, a “patient” refers to any warm-blooded animal, preferably a human. A patient may be afflicted with a disease, or may be free of detectable disease. Accordingly, GOLPH3 or an immunoreactive polypeptide thereof, may be used to treat cancer or to inhibit the development of cancer.

In accordance with this method, the protein, polypeptide or DNA is generally present within a pharmaceutical composition and/or a vaccine. Pharmaceutical compositions may comprise the full length protein or one or more immunogenic polypeptides, and a physiologically acceptable carrier. The vaccines may comprise the full length protein or one or more immunogenic polypeptides and a non-specific immune response enhancer, such as an adjuvant, biodegradable microsphere (PLG) or a liposome (into which the polypeptide is incorporated).

Alternatively, a pharmaceutical composition or vaccine may contain DNA encoding GOLPH3 or an immunogenic polypeptide thereof, such that the full length protein or polypeptide is generated in situ. In such pharmaceutical compositions and vaccines, the DNA may be present within any of a variety of delivery systems known to those of ordinary skill in the art, including nucleic acid expression systems, bacteria and viral expression systems. Appropriate nucleic acid expression systems contain the necessary DNA sequences for expression in the patient (such as a suitable promoter). Bacterial delivery systems involve the administration of a bacterium (such as Bacillus-Calmette-Guerrin) that expresses an epitope of a breast cell antigen on its cell surface. In one embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., PNAS 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., PNAS 91:215-219, 1994; Kass-Eisler et al., PNAS 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in published PCT application WO 90/11092, and Ulmer et al., Science 259:1745-1749 (1993), reviewed by Cohen, Science 259:1691-1692 (1993).

Routes and frequency of administration, as well as dosage, will vary from individual to individual and may parallel those currently being used in immunotherapy of other diseases. In general, the pharmaceutical compositions and vaccines may be administered by injection (e.g., intracutaneous, intramuscular, intravenous or subcutaneous), intranasally (e.g., by aspiration) or orally. Between 1 and 10 doses may be administered over a 3-24 week period. In one embodiment, 4 doses are administered, at an interval of 3 months, and booster administrations may be given periodically thereafter. Alternative protocols may be appropriate for individual patients. A suitable dose is an amount of polypeptide or DNA that is effective to raise an immune response (cellular and/or humoral) against tumor cells, e.g., kidney tumor cells, in a treated patient. A suitable immune response is at least 10-50% above the basal (i.e. untreated) level. In general, the amount of polypeptide present in a dose (or produced in situ by the DNA in a dose) ranges from about 1 pg to about 100 mg per kg of host, from about 10 pg to about 1 mg, or from about 100 pg to about 1 μg. Suitable dose sizes will vary with the size of the patient, but will typically range from about 0.01 mL to about 5 ml.

GOLPH3 or an immunogenic polypeptide thereof can be used in cell based immunotherapies, i.e., stimulation of dendritic cells with GOLPH3 or fusion with GOLPH3 expressing cells. The modified dendritic cells, once injected into the patient, are a cellular vaccine, where the dendritic cells activate an immune response against the GOLPH3 expressing cancer.

While any suitable carrier known to those of ordinary skill in the art may be employed in the pharmaceutical compositions of this invention, the type of carrier will vary depending on the mode of administration. For parenteral administration, such as subcutaneous injection, the carrier can comprise water, saline, alcohol, a fat, a wax and/or a buffer. For oral administration, any of the above carriers or a solid carrier, such as mannitol, lactose, starch, magnesium stearate, sodium saccharine, talcum, cellulose, glucose, sucrose, and/or magnesium carbonate, may be employed. Biodegradable microspheres (e.g., polyleptic galactide) may also be employed as carriers for the pharmaceutical compositions of this invention. Suitable biodegradable microspheres are disclosed, for example, in U.S. Pat. Nos. 4,897,268 and 5,075,109.

Any of a variety of non-specific immune response enhancers may be employed in the vaccines of this invention. For example, an adjuvant may be included. Most adjuvants contain a substance designed to protect the antigen from rapid catabolism, such as aluminum hydroxide or mineral oil, and a nonspecific stimulator of immune response, such as lipid A, Bordella pertussis or Mycobacterium tuberculosis. Such adjuvants are commercially available as, for example. Freund's Incomplete Adjuvant and Complete Adjuvant (Difco Laboratories. Detroit, Mich.) and Merck Adjuvant 65 (Merck and Company, Inc., Rahway, N.J.).

This invention is further illustrated by the following examples which should not be construed as limiting. The contents of all references, patents and published patent applications cited throughout this application, as well as the Figures, are incorporated herein by reference.

### EXAMPLES

### Example 1

### Materials and Methods used in Examples 2-9

A. Cell Lines

All cell lines were propagated at 37° C. and 5% CO₂ in humidified atmosphere in RPMI 1640 Medium (Invitrogen, Carlsbad, Calif.) supplemented with 10% heat-inactivated fetal bovine serum (FBS). CRL-5889, SK-MEL-5, 1205LU, A549 and 293T were obtained from the American Type Culture Collection. MALME-3M was obtained from the NCl cell line panel of the National Cancer Institute-Division of Cancer Treatment and Diagnosis repository. Sbc12, WM239A and hTERT/CDK4(R24C)/p53DD/BRAF^{V600E} melanocytes (HMEL) have been described before (Satyamoorthy et al. (1997) Melanoma Res 7 Suppl 2: S35-S42; Garraway et al., (2005) Nature 436: 117-122).

B. Plasmids, Retroviral Transduction, and siRNA Transfection

The retroviral HA-GOLPH3 expression construct, pBABE-HA-GOLPH3, was constructed by subcloning PCR-generated GOLPH3 ORF (NM_{—}022130) into pBABE-puro-HA (Addgene). The GOLPH3 siRNA resistant construct pBABE-HA-GOLPH3(siRes), which encodes wild-type GOLPH3 protein with nucleotide sequence mutated to resist siRNA against GOLPH3 (si #3), was constructed through site-directed mutagenesis of the pBABE-HA-GOLPH3 vector using primers 5′ tgtatgttaattgaattagcattgaggggtagattgcaactagaggcttgtggaatgagacg and 5′ cgtctcattccacaagcctctagttgcaatctacccctcaatgctaattcaattaacataca. pEF-Dest51-GOLPH3, pLenti4/TO/V5-DEST-GOLPH3 and pLenti6/V5/DEST-VPS35 were constructed via Gateway recombination cloning (Invitrogen) into pEF-Dest51, pLenti4/TO/V5-DEST and pLenti6/V5/DEST (Invitrogen), respectively, using a pDONR223-GOLPH3 and pDONR223-VPS35 entry clone (CCSB, DFCI, Boston, Mass.) according to the manufacturer's protocol. The yeast bait construct, pGBKT7-GOLPH3, was constructed by inserting the GOLPH3 fragment from pBABE-HA-GOLPH3 into the pGBKT7 bait vector (Clontech). The tet-inducible GOLPH3 cell line, HMEL-tet-GOLPH3, was created using the T-Rex lentiviral expression system (Invitrogen) and pLenti4/TO/V5-DEST-GOLPH3 according to the manufacturer's protocol. Lentivirus and retrovirus were prepared by co-transfecting 293T cells with the above-mentioned vector backbones and standard virus packaging systems for subsequent collection of viral supernatants. All over-expression studies were performed with newly-transduced stable cells lines. In HMEL-tet-GOLPH3, GOLPH3 expression was stimulated by the addition of 2 μg/ml doxycycline for 48 hrs for all assays.

For small interfering RNA (siRNA) experiments, cells were seeded to approach 80% confluence at the time of with Lipofectamine 2000 (Invitrogen) according to the manufacturer's protocol. Transfections were performed with Block-It (Invitrogen) or siRNA (Dharmacon, Lafayette, Colo.) targeting GOLPH3 (denoted siGOLPH3: siRNA pool, M-006414-00; si #1, D-006414-01, si #2, D-006414-02, si #3, D-006414-03; si #4, D-006414-04), SUB1 (denoted siSUB1: siRNA pool, M-009815-00) or non-targeting control (denoted siNT: D-001210-02-20). Cells were incubated 48-72 hrs prior to harvest for all assays. Unless otherwise indicated, GOLPH3 knockdown experiments were conducted using siRNA #3. For soft agar colony formation, cells were plated 24 hrs following siRNA transfection.

C. Cross-Tumor aCGH and Expression Analysis

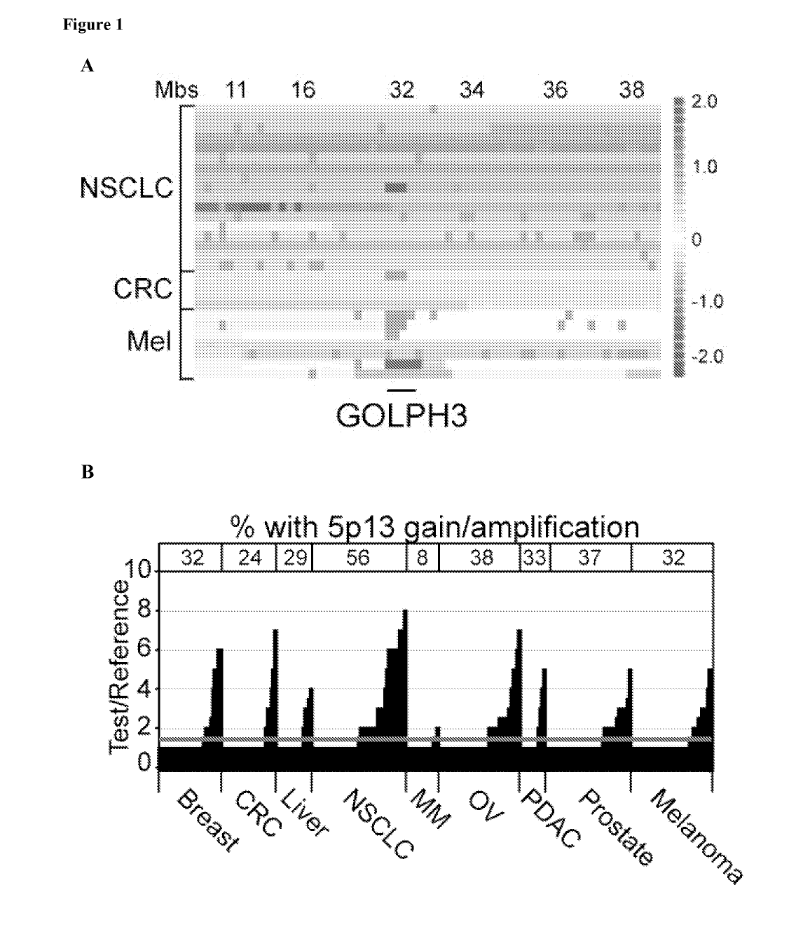

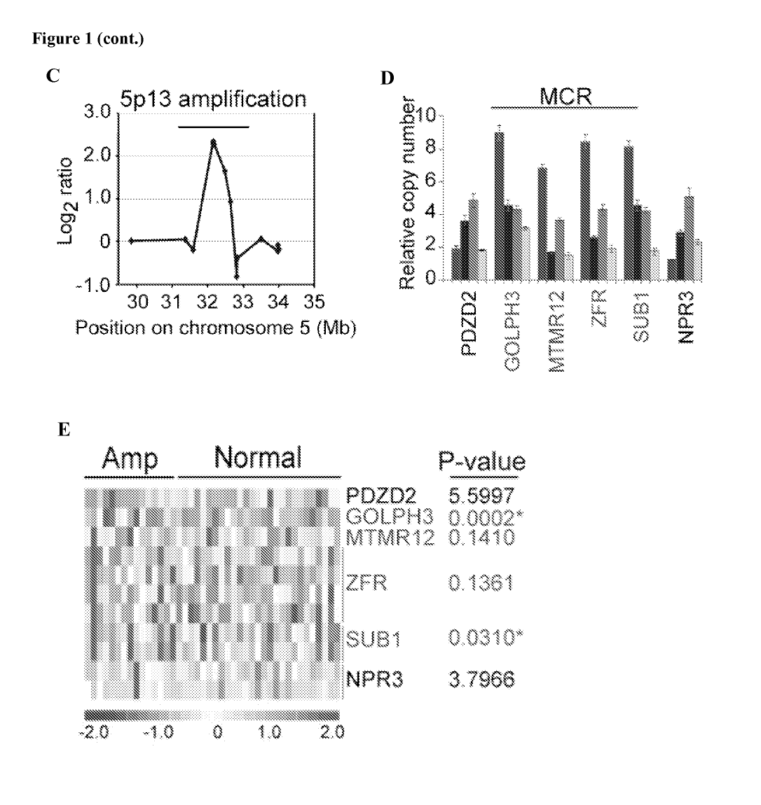

Cross-tumor aCGH analysis of malignant melanoma, non-small cell lung cancer (NSCLC) and colon adenocarcinoma (CRC) was performed as described (Maser et al., (2007) Nature 447: 966-971) using melanoma and CRC data previously submitted to GEO (accession numbers GSE7606 and GSE7604, respectively). The NSCLC dataset has been previously described (Tonon et al., (2005) Proc Natl Acad Sci U.S.A. 102: 9625-9630) and can be found on the world wide web at genomic.dfci.harvard.edu. The number of 5p13 amplifications identified in array-CGH profiles were as follows: Melanoma, 6 (2 focal) present among 88 tumor profiles; Non-Small Cell Lung Cancer, 18 (1 focal) present among 67 profiles (15 cell lines and 52 tumors); and Colorectal cancer, 4 present among 81 profiles (38 cell lines and 43 tumors). For representative 5p13-containing tumor specimen C27 depicted in FIG. 1C, Y-axis is log₂ ratio compared to reference sample (pooled normal human DNA) and X-axis denotes position on chromosome 5. For expression analysis of MCR resident genes, NSCLC expression data accompanying array-CGH profiles was analyzed. Among the 42 samples with both array-CGH 22K profiles and Affymetrix HGU133plus2 profiles available, 14 contained the 5p13 amplification event. Expression values for each gene (values from multiple probes for same gene were averaged) from two groups (with amplification or without amplification) were compared by two-sample t test and the significance level was adjusted with Boferroni correction.

D. TMA-FISH

The following tissue microarrays were purchased from Cybrdi (Frederick, Md.): CC04-01-004 lung carcinoma, CC11-11-005 ovarian carcinoma, CC08-01-002 breast carcinoma, CC05-21-001 colon adenocarcinoma, CC03-01-003 liver carcinoma and CC19-11-007 prostate carcinoma. Multiple myeloma tissue microarray was from TriStar (Rockville, Md.). PA802 pancreatic carcinoma and ME1001 melanoma tissue microarrays were from US Biomax. Fluorescence in situ hybridization was prepared following standard protocols. BAC RP11-437P15 was used to mark the region of gain at 32 MB on chromosome 5 (5p13). Centromere-specific CEP1 probe (Abbott Laboratories) was served as a ploidy reference. FISH signals evaluation and acquisition were performed manually using filter sets and software developed by Applied Spectral Imaging. Signal to reference ratio greater than 1.5 was considered as gain; ratio above 2.5 as a high amplification level.

E. TMA-IHC and Automated Quantitative Analysis (AQUA®)

Arrays were deparaffinized with xylene, rehydrated and antigen-retrieved by pressure cooking for 20 minutes in citrate buffer (pH=6). Slides were pre-incubated with 0.3% bovine serum albumin (BSA) in 0.1M tris-buffered saline (TBS, pH=8) for 30 minutes at room temperature. Lung cancer TMAs were then incubated overnight with a cocktail of either the mTOR primary antibody diluted 1:200 (rabbit monoclonal, clone 7C10, Cell Signaling Technology) and a mouse monoclonal anti-human cytokeratin antibody (clone AE1/AE3, M3515, Dako) diluted 1:100 in BSA/TBS or the phospho-S6K^{Thr389} primary antibody diluted 1:200 (mouse monoclonal, clone 1A5, Cell Signaling Technology) and a wide-spectrum rabbit anti-cow cytokeratin antibody (Z0622, Dako) diluted 1:100 in BSA/TBS. For the melanoma cohort a mouse monoclonal S100 antibody (15E2E2, BoGenex) and a rabbit polyclonal S100 antibody (Z0311, Dako) both diluted 1:100 in BSA/TBST were used instead of the mouse and rabbit cytokeratin respectively. This was followed by an 1-hour incubation with Alexa 546-conjugated goat anti-mouse secondary antibody (A11003, Molecular Probes) diluted 1:100 in rabbit EnVision reagent (K4003, Dako) and Alexa 546-conjugated goat anti-rabbit secondary antibody (A11010, Molecular Probes) diluted 1:100 in mouse EnVision reagent (K4001, Dako) for mTOR and phospho S6K respectively. Cyanine 5 (Cy5) directly conjugated to tyramide (FP1117, Perkin-Elmer) at a 1:50 dilution was used as the fluorescent chromagen for target detection. Prolong mounting medium (ProLong Gold, P36931, Molecular Probes) containing 4′,6-Diamidino-2-phenylindole (DAPI) was used to identify tissue nuclei. Serial sections of a smaller TMA consisting of 30 lung cancer specimens (NSCLC “test” array) were stained aside both cohorts to confirm assay reproducibility. H1299 and A431 cells were used as positive controls as indicated by the manufacturer. Negative control sections, in which the primary antibody was omitted, were used for each immunostaining run.

Automated Quantitative Analysis (AQUA®) allows exact measurement of protein concentration within subcellular compartments, as described in Camp et al., (2002) Nat. Med. 8, 1323-1327. In brief, a series of high resolution monochromatic images were captured by the PM-2000 microscope. For each histospot, in- and out-of-focus images were obtained using the signal from the DAPI, cytokeratin and S100-Alexa 546 (for lung cancer and melanoma respectively) and mTOR/phospho S6K-Cy5 channel. mTOR and phospho S6K were measured using a channel with emission maxima above 620 nm, in order to minimize tissue autofluorescence. Tumor was distinguished from stromal and non-stromal elements by creating a tumor “mask” from the cytokeratin and S100 signal for lung cancer and melanoma specimens respectively. This created a binary mask (each pixel being either “on” or “off”) on the basis of an intensity threshold set by visual inspection of histospots. AQUA® score of the protein of interest in each subcellular compartment was calculated by dividing the signal intensity (scored on a scale from 0-255) by the area of the specific compartment. Specimens with less that 5% tumor area per spot were not included in automated quantitative analysis for not being representative of the corresponding tumor specimen.

For statistical analysis, Pearson's correlation coefficient (R) was used to assess the correlation between log normalized mTOR and pS6K AQUA® scores as well as the same cores on serial cuts of the NSCLC “test” array. Evaluation of the inter-array reproducibility did not reveal significant differences between serial sections of the NSCLC “test” array (Pearson's R=0.95, p<0.0001). Ratios of cytoplasmic to nuclear expression for mTOR and pS6K were calculated in order to normalize for individual variation between groups and compared to GOLPH3 gene copy number. The association between mTOR, pS6K AQUA® scores and GOLPH3 gene copy number (as determined by 5p13 FISH) and clinicopathologic parameters (age, gender, histological type and tumor differentiation) were analyzed using Spearman rank test. GOLPH3 copy number status was also binarized based on FISH signals as normal (<1.5) and gain (>=1.5) and correlated as a non-continuous variable with all other parameters using Pearson's correlation.

F. Quantitative PCR and Copy Number

Genomic DNA was prepared from cell lines and melanomas with the Wizard kit (Promega), and total RNA was isolated using Trizol reagent (Invitrogen) and RNeasy columns (Qiagen). DNA copy numbers and relative expression levels were determined by real-time PCR using SYBR green I (Qiagen) detection chemistry and the Stratagene MX3000p detection system according to the manufacturer's protocol. The comparative cycle threshold method was used to quantify target gene or mRNA copy numbers in the samples. For quantification of gene copy numbers, copy numbers in tumor DNA were compared to copies in normal human control DNA (Promega). The DNA copy number normalization reference was Line-1 DNA copy number. For delimitation of chromosome 5p13 amplicon boundaries by genomic qPCR presented in FIG. 1D, samples include tumor specimens C27 (primary melanoma, red), Cl (primary melanoma, blue), CRL-5889 (NSCLC cell line, green) and Sbc12 (melanoma cell line, yellow).

G. Anchorage Independent Growth

Soft-agar assays were performed on 6-well plates in triplicate. For each well, 1×10⁴ cells were mixed thoroughly in cell growth medium containing 0.4% SeaKem LE agarose (Fisher) in RPMI+10% FBS, followed by plating onto bottom agarose prepared with 0.65% agarose in RPMI+10% FBS. Each well was allowed to solidify and subsequently covered in 1 ml RPMI+10% FBS, which was refreshed every 4 days. Colonies were stained with 0.05% (wt/vol) iodonitrotetrazolium chloride (Sigma) and scanned at 1200 dpi using a flatbed scanner, followed by counting and two-tailed t-test calculation using Prism 4 (Graphpad).

H. Proliferation Assays

Proliferation assays were performed on 12-well plates in triplicate using 7×10³ (A549) or 1×10⁴ (CRL-5889 and 1205LU) cells per well. Cells were fixed in 10% formalin in PBS and stained with crystal violet at 24 hr increments starting after cell adherence (T0). At the conclusion of the assay, crystal violet was extracted using 10% acetic acid and measured and assessed for absorbance at 595 nm (ABS₅₉₅).

I. Focus Formation Assay

Ink4a/Arf-deficient primary MEFs were plated in DMEM containing 10% FBS at a density of 8×10⁵ cells/10-cm 16 hrs prior to transfection. For RAS cooperation, 1.5 ug HRAS^{V12} vector was co-transfected with 6.5 ug pEF-Dest51-LacZ control vector, MYC or pDest51-GOLPH3 using Lipofectamine-2000 (Invitrogen) following the manufacturer's instructions. The total amount of transfected DNA was kept constant at 7.5 ug using pDest51-LacZ, and transfections were done in duplicate three times. At 48 hrs post-transfection, each transfected 10-cm plate was equally split into three 10-cm plates and incubated for 10 days during which media was refreshed twice. Cells were washed, fixed in 10% formalin and stained with Giemsa solution (Sigma) for 10 min. at room temperature for foci quantitation. Two-tailed t-test calculations were performed using Prism 4 (Graphpad).

J. Yeast Two-Hybrid Interaction Screening

A pre-transformed human fetal brain cDNA library (Clontech) was screened (1×10⁶ clones) using the AH109 yeast reporter strain and the MATCHMAKER Two-Hybrid System 3 (Clontech) according to the manufacturer's instructions. Plasmid DNA from 119 potential positive clones was isolated after transformation into Escherichia coli strain DH5α, followed by DNA sequencing using the provided prey vector-specific primers. Informative sequencing data was obtained for 102 of the 118 clones, 44 of which contained partial to full-length coding sequence and were further considered for downstream analysis. GOLPH3-dependency was assessed by serial loss passaging the positive clones in the absence of vector selection, followed by replica plating VPS35-expressing AH109 cells to SC-LT (to confirm absence of the GOLPH3 bait vector) and to SC-L-H-A+XαGAL (to confirm loss of reporter activation).

K. Xenograft Studies

WM239A and A549 cells transduced with either empty vector or HA-GOLPH3 retrovirus (pBABE-HA-GOLPH3) were stably selected and subcutaneously implanted in female nude animals (Taconic) at 1.0×10⁶ and 2.5×10⁶ cells/site on both flanks, respectively, mixed 1:1 with Matrigel (BD Bioscience). WM239A- and A549-derived tumors were isolated at 22 days and 45 days, respectively, and measured for tumor volume. Two-tailed t-test calculations were performed using Prism 4 (Graphpad).

For in vivo rapamycin studies, melanoma 1205LU and WM239A cells verified to stably-express empty vector or GOLPH3 were subcutaneously implanted into female nude animals (Taconic) at 5.0×10⁵ cells/site on both flanks. Tumors were allowed to reach approximately 100 mm³, after which time animals were randomized into separate cohorts for treatment with vehicle [5% (vol/vol) PEG400.5% (vol/vol) Tween-80) or rapamycin (6 mg/kg; LC Laboratories; 50 mg/ml stock prepared in 100% ETOH and diluted fresh in vehicle solvent for treatment) by intraperitoneal injection every other day. Tumor volumes and body weights were measured upon drug administration. Tumor volume was determined by measuring in two directions with vernier calipers and formulated as tumor volume=(length×width)/2. Growth curves were plotted as mean change in tumor volume for each group, where mean change indicates change in tumor volume at a given time point minus tumor volume at time of initial dose administration. Endpoint scatter plots were plotted as proportion of final dose endpoint tumor volume over respective tumor baseline starting volume. Percent tumor growth inhibition was determined as (1-(T/N))×100, where T is the mean change in tumor volume of the treated group and N is the mean change in tumor volume of the control group at the assay endpoint. Two-tailed t-test calculations were performed using Prism 4 (Graphpad).

L. Cell Size Determination

A Becton Dickinson FACScan flow cytometer with Cell Quest software was used to determine relative changes in cell size as assessed by forward scatter differences. A549 cells were seeded to 60-mm dishes for siRNA transfection the following day as indicated above, followed by incubation overnight. Cells were split to 10-cm dishes with or without 25 nM rapamycin (EMD Bioscience) and harvested for flow cytometry by ethanol fixation following 60 hrs post-transfection. For FACS analysis, 10,000 single cells were collected and assessed for forward scatter.

M. Immunoblotting, Immunofluorescence and Co-Immunoprecipitation Analyses

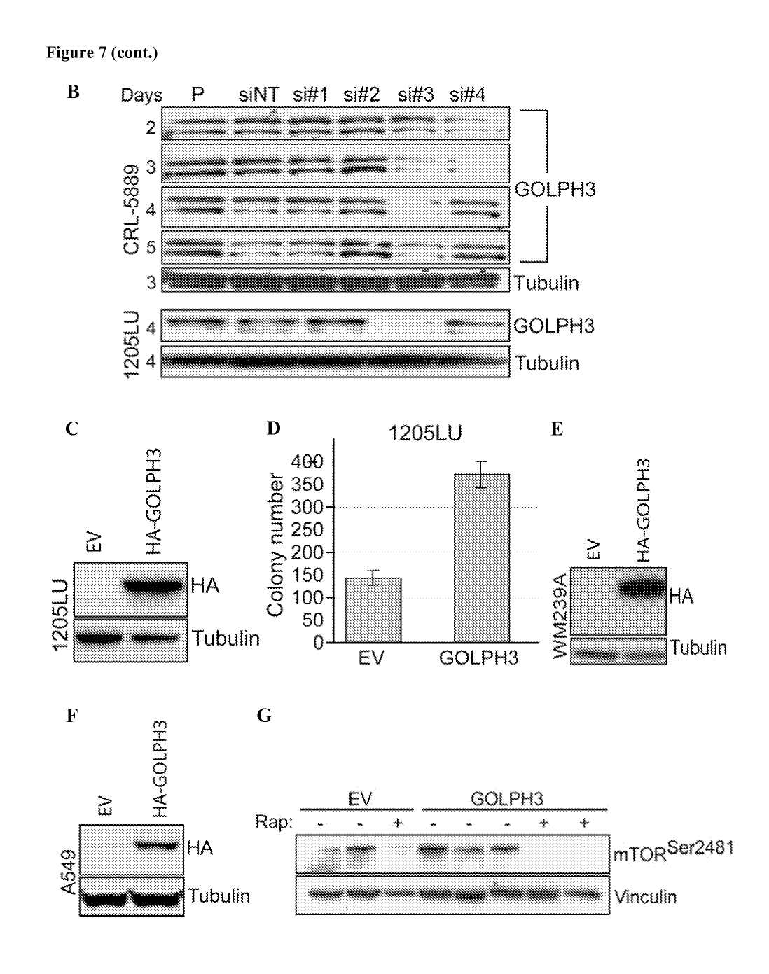

For EGF stimulation assays, cells were serum starved for 24 hrs followed by treatment as indicated with 100 ng/ml EGF (Invitrogen). For immunoblotting, cells were washed in 2× in PBS and lysed using RIPA buffer (Boston BioProducts) containing 1 mM PMSF, 1× Protease Inhibitor Cocktail (Sigma) and 1× Phosphatase inhibitor (Calbiochem) for separation on NuPAGE 4-12% Bis-Tris gels (Invitrogen) and blotted onto PVDF (Millipore). The following antibodies were used for immunoblotting following the manufacture's recommendations: phospho-p70 S6K (Thr389; 1:1000; Cell Signaling), p70 S6K (1:1000; Cell Signaling), phospho-AKT (Ser473, 1:1000; Cell Signaling), AKT (1:1000; Cell Signaling), mTOR (Ser2481, 1:1000; Cell Signaling), alpha-tubulin (1:20,000; Sigma), vinculin (1:5,000; Santa Cruz), phospho-4E-BP1 (Thr37/46, 1:1000; Cell Signaling), phospho-PDK1 (Ser241, 1:1000; Cell Signaling), PTEN (1:750; Cell Signaling), phospho-p27Kip (Thr157; 1:500; R&D Systems), phospho-MEK1/2 (Ser217/221; 1:1000; Cell Signaling), phospho-p44/42 (Erk1/2; Thr202/Tyr204, 1:1000; Cell Signaling), V5 (1:5,000; Invitrogen), and HA (1:1000; Cell Signaling). Rabbit anti-GOLPH3 (BB; 1:1000) used in FIG. 7B was obtained as a gift from JJ Bergeron, McGill University, Montreal, Quebec. All other GOLPH3 immunoblotting was performed using mouse anti-GOLPH3 (C19; 1:1000) prepared by the Dana Farber/Harvard Cancer Center Monoclonal Antibody Core Facility.

For co-immunoprecipitation studies, parental A549 cells or 293T cells co-transfected with the combinations of pBABE-GOLPH3-HA, pLenti6N5/DEST-VPS35 or the corresponding empty vectors were used. Cytosolic lysates from these A549 cells or transfectants were prepared with hypotonic buffer {10 mM Tris (pH 7.6), 10 mM KCl, 5 mM MgCl2, 1% NP40 and protease inhibitors} 48 hrs post-transfection (for 293T cells), and protein lysates were adjusted to 50 mM Tris (pH 7.6) and 150 mM NaCl for immunoprecipitation using either anti-V5, anti-HA or anti-GOLPH3 antibody overnight at 4° C. with rocking Protein G beads (Roche) were added to the lysate-antibody mix following the manufacture's recommendations and incubated for an additional 3 hours at 4° C. with rocking Immunoprecipitants were washed 3× for 20 min with either low stringent buffer for anti-V5 IP (PBS plus 0.1% NP40 and 0.05% Nadeoxycholate) or high stringency buffer for anti-HA and anti-GOLPH3 IP (RIPA buffer with 500 mM NaCl). Immunoprecipitation complexes were eluted by the addition of SDS loading buffer after centrifugation and resolved on NuPAGE 4-12% Bis-Tris gels (Invitrogen) for immunoblotting analysis for Golph3 (anti-HA or anti-GOLPH3) and Vps35 (anti-V5).

For immunofluorescence studies, cells were cultured on cover slips, followed by fixation for 15 min at RT in 4% paraformaldehyde in PBS, permeabilization for 10 min RT in 0.1% Triton X-100/PBS and blocking 1 hr RT in 10% goat or donkey serum/PBS. Slides were incubated 1 hr RT with the following antibodies: mouse anti-HA (1:100, Cell Signaling); rabbit anti-TGN46 (1:500; Abcam), goat anti-VPS35 (1:200; Abcam) and mouse anti-GOLPH3 (1:300). Slides were stained for 1 hour RT with the corresponding Alexa Flour secondary antibodies (Invitrogen). Cover slips were stained with DAPI (Sigma) and mounted with mounting medium. Microscopic images were obtained with a Nikon inverted Ti microscope equipped with Yokogawa spinning disk confocal/TIRF system and a Hamamatsu Orca ER firewire digital CCD camera using constant exposure times for each channel in individual experiments. Images were compiled and false-colored with Adobe Photoshop using identical settings for each color. Magnification is 100× unless otherwise indicated.

### Example 2

### GOLPH3 Copy Number Aberrations in Various Tumors

The human cancer genome harbors numerous chromosomal alterations resulting in irreversible numerical and structural aberrations affecting a plethora of genetic elements, including causal events that can activate oncogenes and inactivate tumor suppressor genes, as well as genomic bystanders that are biologically neutral. Distinguishing the causal events from noise is a central challenge facing genomic science today. Triangulation across model systems has proven to be a powerful filter for prioritizing evolutionarily conserved syntenic events likely to be biologically important (Kim et al, (2006) Cell 125: 1269-1281; Zender et al., (2006) Cell 125: 1253-1267; Maser et al., (2007) Nature 447: 966-971). By that same logic, it was hypothesized that genomic alterations observed in cancers of different tissue lineages are more likely to be pathogenetically relevant and functionally robust.

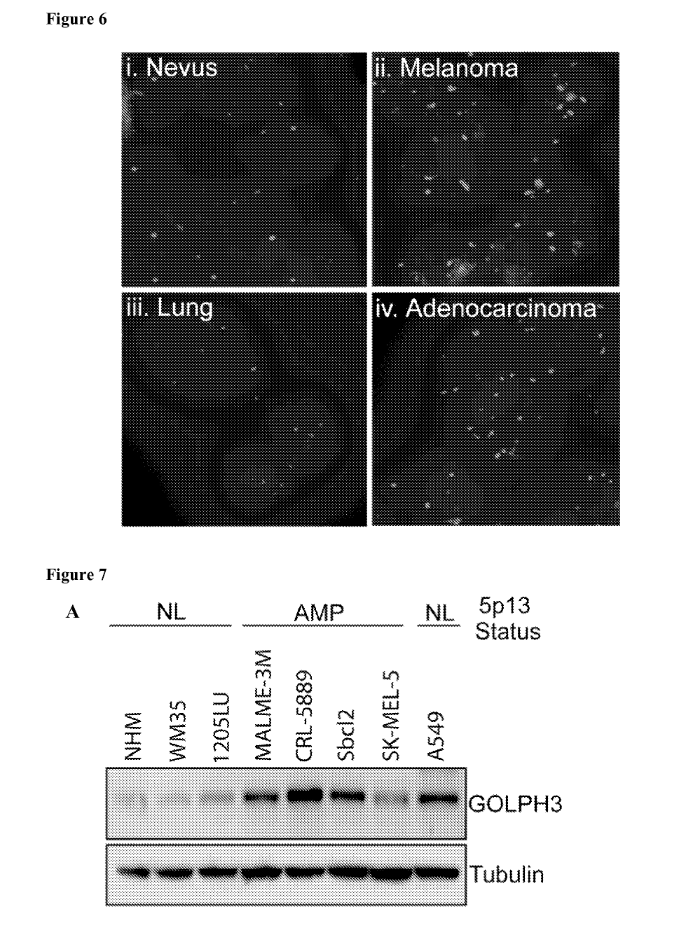

Array-CGH analysis of 83 melanoma specimens revealed a focal amplification within a larger 5p13 regional copy number gain, which was also present in non-small cell lung cancer and colon adenocarcinomas (FIG. 1A), prompting a broad survey by fluorescence in situ hybridization (FISH). Analysis on tumor tissue microarrays (TMA) containing 307 cores of diverse tumor types showed that 5p13 gain was present significantly in all tumor types surveyed, including 56% (27 of 48) of non-small cell lung carcinoma (NSCLC) cores, 38% (18 of 48) of ovarian carcinoma cores, 37% (16 of 43) of prostate cancer cores as well as 32% (12 of 38) of melanoma cores (FIG. 1B, FIG. 6, Table 1). Quantitative real-time PCR across the 5p13 region in informative tumor samples delimited a 0.8 MB minimal common region (MCR) encompassing four resident annotated genes (FIGS. 1C and 1D). Given that copy number aberration (CNA) is a mechanism to drive deregulated gene expression, we next investigated the expression pattern of these resident genes in a NSCLC collection with matched expression and array-CGH profiles were also investigated. As shown in FIG. 1E, only GOLPH3 and SUB1, but not MTMR12 and ZFR, showed statistically significant correlation between expression level and copy number status, thereby pointing to GOLPH3 and SUB1 as viable candidate target(s) of this amplification. Moreover, increased copy number variation was further observed in germline DNA of several cohorts, including the presence of GOLPH3 copy number variation in approximately 1.8% of the HapMap cohort (see the world wide web at hapmap.ncbi.nlm.nih.gov) and in approximately 4% of the cancer patients in the Cancer Genome Atlas (see the world wide web at cancergenome.nih.gov). Accordingly, somatic and/or germline copy number variations in GOLPH3 (e.g., increases in GOLPH3 copy number) is believed to be predictive of increased risk for cancer (see, as non-limiting examples, the cancer types listed in Table 1).

TABLE 1
Summary of TMA-FISH analysis for 5p13 amplification

| | Number | | |
|---|---|---|---|
| | Informative | 5p13 Status | |
| Cancer Type | Cores | Gain* | Amplification** |
| Lung Carcinoma | 48 | 27 (56.3%) | 16 (33.3%) |
| Ovarian Carcinoma | 48 | 18 (37.5%) | 12 (25.0%) |
| Pancreatic Carcinoma | 12 | 4 (33.3%) | 3 (25.0%) |
| Liver Carcinoma | 17 | 5 (29.4%) | 4 (23.5) |
| Breast Carcinoma | 31 | 10 (32.3%) | 6 (19.4%) |
| Prostate Carcinoma | 43 | 16 (37.2%) | 8 (18.6%) |
| Melanoma | 38 | 12 (31.6%) | 7 (18.4%) |
| Colon Carcinoma | 33 | 8 (24.2%) | 4 (12.1%) |
| Multiple Myeloma | 37 | 3 (8.1%) | 0 |

*Gain = signal to reference ratio 1.5 to 2.5
**Amplification = signal to reference ratio >2.5

### Example 3

### GOLPH3 Effects on Cellular Transformation as Assessed by Loss-of-Function Analysis

To assess the cancer-relevance of GOLPH3, SUB1 or both, knockdown assays using pooled siRNAs (Table 2) were performed to gauge the dependence of human tumor (NSCLC and melanoma) cell lines on either gene for their transformed phenotype relative to the underlying copy number status and overall protein expression level (FIG. 7A). Knockdown of GOLPH3 resulted in significant loss of anchorage independent growth in CRL-5889 (NSCLC), Sbcl2 and SK-MEL-5 (melanoma), three human cancer cell lines with 5p13 amplification and high expression level. However, a similar level of knockdown in 1205LU, a melanoma cell line without the 5p13 CNA and with low protein expression, resulted in minimal effect on anchorage independence (Table 2). In contrast, equally effective knockdown of SUB1 in the 5p13-amplified tumor lines had either no or relatively modest effects on anchorage independence.

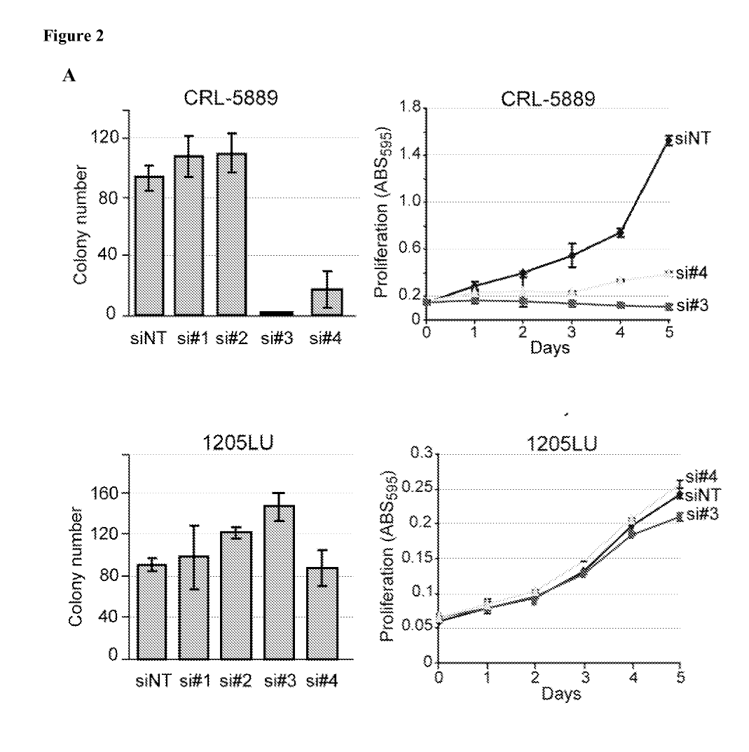

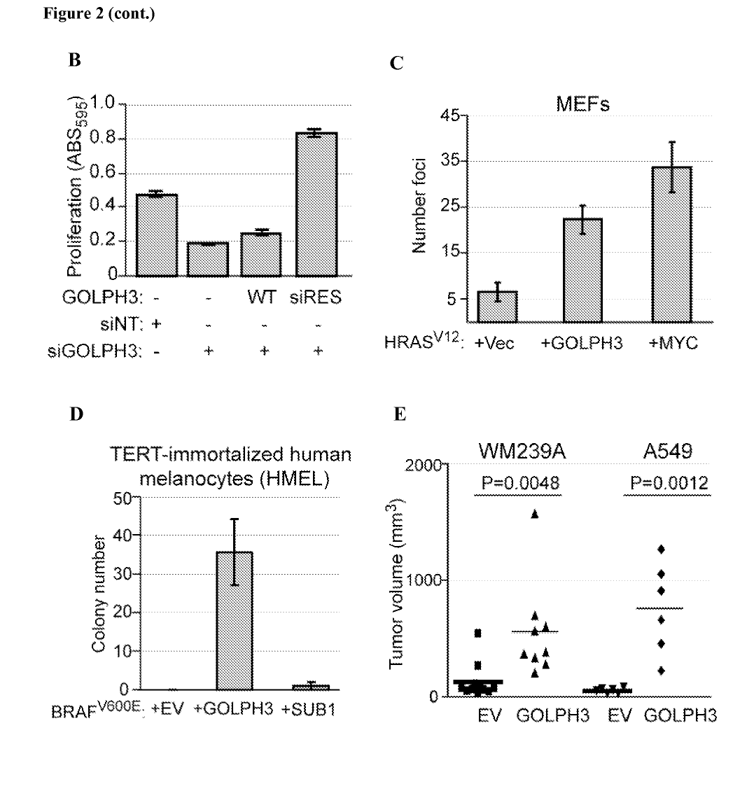

To confirm that the observed knockdown activity was not due to an off-target effect of the GOLPH3 siRNA, the siRNA pool was deconvoluted and verified that two of the four independent siRNA duplexes (siRNAs #3 and #4) were effective at knocking down GOLPH3 (FIG. 7B), which led to potent suppression of soft agar growth and inhibition of proliferation in 5p13-amplified CRL-5889 cells (FIG. 2A). Similarly effective knockdown in 1205LU without 5p13 gain and low GOLPH3 expression showed minimal effect, indicating that acute GOLPH3 depletion was not generally toxic to all cells (FIG. 2A and FIG. 7B). Importantly, specificity of siRNA#3 against GOLPH3 was further documented by rescue of A459 proliferation by a GOLPH3 cDNA engineered to be insensitive to siRNA#3 (siRES) (FIG. 2B). Together, these genetic loss-of-function studies using RNAi-mediated knockdown pointed to GOLPH3 as the likely functionally active target of this amplification.

TABLE 2
Summary of soft agar colony counts (SA#) and corresponding
siRNA KD (% KD) of GOLPH3 and SUB1 in the indicated cell lines
with amplified (AMP) or normal (NL) GOLPH3 copy number.

| | siNT | siGOLPH3 | | siSUB1 | |
|---|---|---|---|---|---|
| Cell line | SA# | SA# | % KD | SA# | % KD |
| CRL-5889 | 183 ± 2 | 19 ± 1 | 95 ± 1 | 120 ± 2 | 99 ± 1 |
| SK-MEL-5 | 32 ± 5 | 12 ± 5 | 90 ± 3 | 31 ± 5 | 90 ± 3 |
| Sbcl2 | 117 ± 2 | 9 ± 1 | 92 ± 5 | ND | ND |
| 1205LU | 87 ± 1 | 80 ± 3 | 85 ± 1 | 45 ± 1 | 89 ± 3 |

ND = not determined.

### Example 4

### GOLPH3 Effects on Cellular Transformation as Assessed by Gain-of-Function Analysis

To reinforce the GOLPH3 loss-of-function studies, the impact of ectopic GOLPH3 expression in a number of model systems was assessed. GOLPH3 was capable of effecting malignant transformation of both primary non-transformed mouse and human cells. Specifically, in the classical co-transformation assay, GOLPH3 cooperated with activated HRAS^{V12} to increase transformed focus formation in Ink4a/Arf-deficient primary mouse embryonic fibroblasts (MEF) (FIG. 2C; 3.4-fold increase relative to HRAS^{V12} alone). In primary human cells, GOLPH3 cooperated with oncogenic BRAF^{V600E} in TERT-immortalized human melanocytes (hereafter referred to as “HMEL”) (Garraway et al. (2005) Nature 436: 117-122) to confer anchorage independent growth in soft agar, whereas SUB1 showed no transforming activity in this system (FIG. 2D). Similar activity was also observed in the 1205LU melanoma cell line (no 5p13 amplification, low GOLPH3 expression), wherein GOLPH3 over-expression (FIG. 7C) enhanced anchorage independent growth and cell proliferation in vitro (FIG. 7D). Lastly, GOLPH3 over-expression (FIGS. 7E-7F) significantly enhanced xeno-transplanted tumor growth (FIG. 2E) of human melanoma (WM239A) and NSCLC (A549) cell lines, both without 5p13 amplification. This series of reinforcing knockdown and over-expression studies demonstrates that GOLPH3 is a bona fide oncogene with potent transforming activity.

### Example 5

### GOLPH3 Localizes to the Golgi

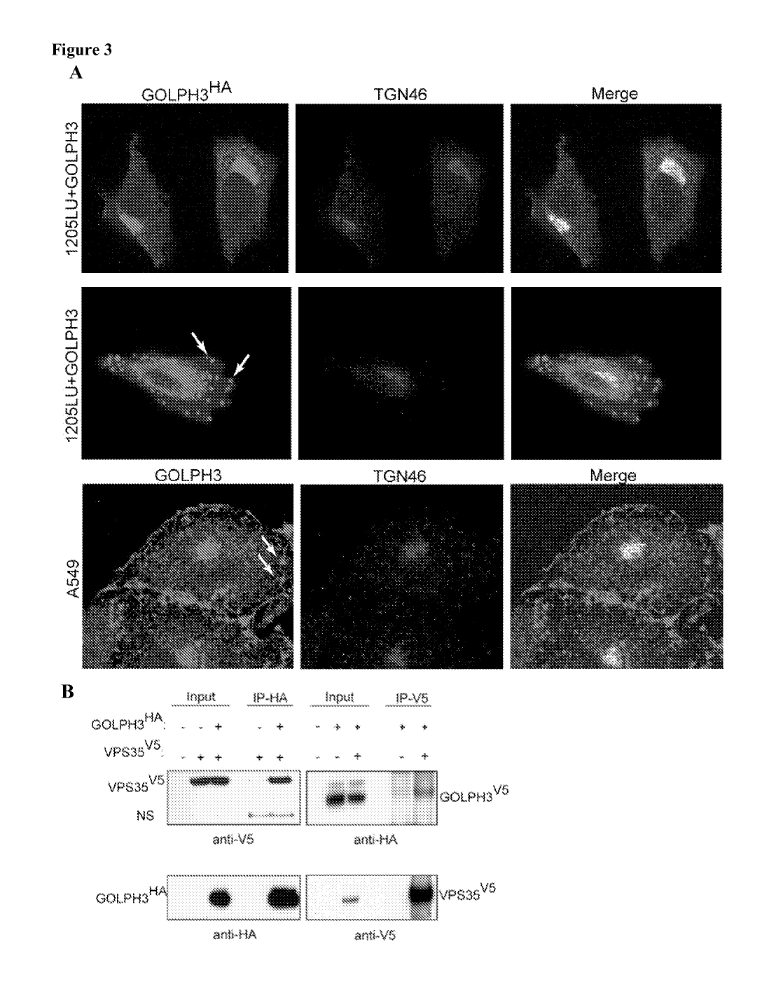

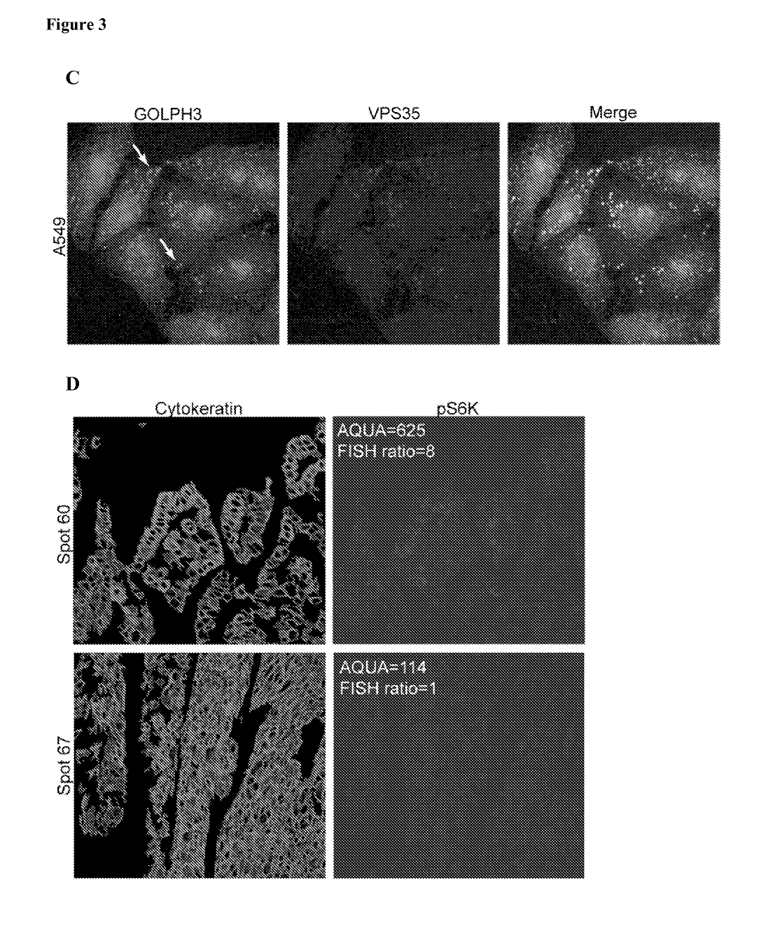

GOLPH3 (alias GPP34; GMx33) was initially identified as a peripheral membrane protein localized to the trans-Golgi Network (TGN) (Wu et al. (2000) Traffic 1: 963-975; Bell et al., (2001) J Biol Chem 276: 5152-5165). Subsequent work with the rat homolog, GMx33, revealed that the protein is dynamically associated with the trans-Golgi matrix, rapidly moving from the TGN to the cytosol with localization in endosomes and at the plasma membrane (Snyder et al. (2006) Mol. Biol. Cell 17: 511-524). As a class, TGN-localizing proteins have not been directly implicated in cancer pathogenesis. Thus, it was first confirmed herein by confocal microscopy that both exogenously-expressed and endogenous human GOLPH3 indeed co-localized to the Golgi apparatus via co-immunofluorescence with the TGN marker, TGN46, and to endosome-like structures (FIG. 3A; Snyder et al. (2006) Mol. Biol. Cell 17: 511-524).

### Example 6

### GOLPH3-Interactors Identified by Yeast-2-Hybrid Screening

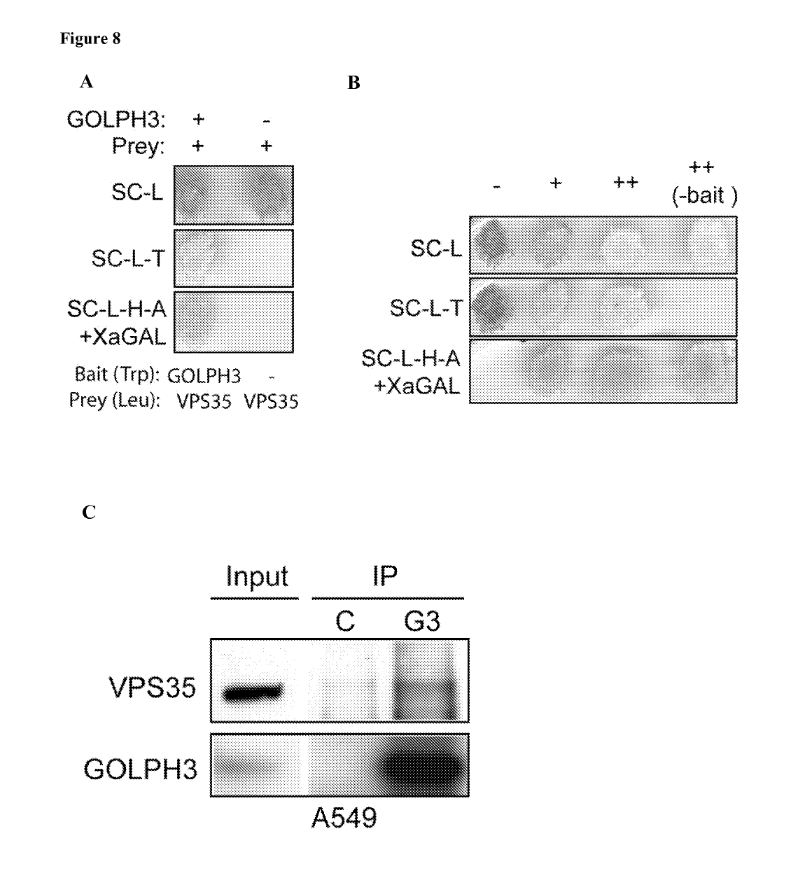

To gain mechanistic insights into the biological functions of GOLPH3, GOLPH3-interacting proteins were screened using the yeast two-hybrid system (Table 3). Most notable among the GOLPH3-interacting proteins was VPS35, a highly conserved member of the cargo-recognition complex of the retromer, which regulates retrograde transport of proteins that include transmembrane receptors from endosomes to the TGN (Bonifacino et al. (2008) Curr. Opin. Cell. Biol. 20: 427-436). After documenting bait-dependent interaction of GOLPH3 with VPS35 in yeast (FIGS. 8A-8B), physical interaction of VPS35 with both exogenously-expressed and endogenous GOLPH3 in human cells was demonstrated by co-immunoprecipitation (FIG. 3B and FIG. 8C). Confocal co-immunofluorescence studies further confirmed co-localization of endogenous VPS35 and GOLPH3 at endosome-like structures (FIG. 3C).

A large-scale chemical genomic profiling screen in S. cerevisiae (Xie et al. (2005) Proc. Natl. Acad. Sci. USA 102: 7215-7220) has found that deletion mutants of VPS35 and VPS29 exhibited altered sensitivity to rapamycin, an inhibitor of TOR signaling, suggesting that the retromer complex might function in the TOR signaling pathway in budding yeast. Thus, it was postulated that GOLPH3 might regulate the mammalian ortholog of TOR (mTOR) thereby contributing to the pro-tumorigenic effects of GOLPH3. This hypothesis is supported by the observation that, in human NSCLC tumor specimens, high 5p13 copy number was associated with increased mTOR expression and elevated phosphorylation of the mTOR substrate, S6 Kinase (S6K) by AQUA® quantitative immunofluorescence (Camp et al. (2002) Nat. Med. 8: 1323-1327; FIG. 3D and Tables 4-5). Specifically, mTOR expression level was associated with cytoplasmic, but not nuclear nor total, phospho-S6K^{Thr389} (pS6K) level (Pearson's R=0.42, p=0.001). When the 5p13 copy number status was binarized into normal (with FISH-determined signal<1.5) and gained (FISH signal>=1.5), a significant correlation with increased mTOR (Spearman's rho=0.475, p=0.04) and cytoplasmic pS6K (Spearman's rho=0.724, p<0.0001) was observed, signifying that 5p13 CNA is positively correlated with mTOR-pS6K activity in the adenocarcinoma subtype of NSCLC. Importantly, even when the 5p13 copy number was treated as a continuous variable, significant correlation was still observed with cytoplasmic pS6K in this subtype of NSCLC (Pearson's R=0.513, p<0.025; Table 4). Taken together, this correlative relationship in human cancers, coupled with the yeast genetic interaction data, supports the hypothesis that GOLPH3 regulates mTOR activity in mammalian cells.

TABLE 3
Summary of GOLPH3-interactors identified by yeast-2-hybrid screening

| | | Gene | |
|---|---|---|---|
| Gene | Description | ID | Accession ID |
| ACTG1 | Actin, beta | 71 | NM_001101 |
| AP1G1 | Adaptor-related protein complex 1, gamma 1 subunit | 164 | NM_001030007 |
| ARF4 | Adp-ribosylation factor 4 | 378 | NM_001660 |
| CAMLG | Calcium modulating ligand | 819 | NM_001745 |
| COL1A2 | Collagen, type i, alpha 2 | 1278 | NM_000089 |
| AF322220 | Eukaryotic translation elongation factor 1 alpha 1 | 1915 | NM_001402 |
| EEF2 | Eukaryotic translation elongation factor 2 | 1938 | NM_001961 |
| EIF4A2 | Eukaryotic translation initiation factor 4a, isoform 2 | 1974 | NM_001967 |
| FHL2 | Four and a half lim domains 2 | 2274 | NM_001039492 |
| FLNA | Filamin a, alpha (actin binding protein 280) | 2316 | NM_001456 |
| FTLL1 | Ferritin, light polypeptide | 2512 | NM_000146 |
| GPM6A | Glycoprotein m6a | 2823 | NM_005277 |
| HSBP1 | Heat shock factor binding protein 1 | 3281 | NM_001537 |
| IFRD1 | Interferon-related developmental regulator 1 | 3475 | NM_001007245 |
| LGALS1 | Lectin, galactoside-binding, soluble, 1 (galectin 1) | 3956 | NM_002305 |
| NDUFA4 | Nadh dehydrogenase (ubiquinone) 1 alpha subcomplex, | 4697 | NM_002489 |
| | 4, 9 kda | | |
| NGFR | Nerve growth factor receptor (tnfr superfamily, member | 4804 | NM_002507 |
| | 16) | | |
| NKX2-2 | Nk2 transcription factor related, locus 2 (drosophila) | 4821 | NM_002509 |
| PEX10 | Peroxisome biogenesis factor 10 | 5192 | NM_153818 |
| PSAP | Prosaposin (variant gaucher disease and variant | 5660 | NM_002778 |
| | metachromatic leukodystrophy) | | |
| S100B | S100 calcium binding protein, beta (neural) | 6285 | NM_006272 |
| SYT4 | Synaptotagmin iv | 6860 | NM_020783 |
| TTC3 | Tetratricopeptide repeat domain 3 | 7267 | NM_001001894 |
| GTF3C5 | General transcription factor iiic, polypeptide 5, 63 kda | 9328 | NM_012087 |
| MAGED1 | Melanoma antigen family d, 1 | 9500 | NM_006986 |
| ELMO1 | Engulfment and cell motility 1 | 9844 | NM_001039459 |
| RANBP9 | Ran binding protein 9 | 10048 | NM_005493 |
| HAX1 | Hcls1 associated protein x-1 | 10456 | NM_006118 |
| CCT7 | Chaperonin containing tcp1, subunit 7 (eta) | 10574 | NM_001009570 |
| ClpX | Clpx caseinolytic peptidase × homolog (E. coli) | 10845 | NM_006660 |
| GABARAP | Gaba(a) receptor-associated protein | 11337 | NM_007278 |
| KCNH3 | Potassium voltage-gated channel, subfamily h (eag- | 23416 | NM_012284 |
| | related), members | | |
| WSB1 | Wd repeat and socs box-containing 1 | 26118 | NM_015626 |
| CARD10 | Caspase recruitment domain family, member 10 | 29775 | NM_014550 |
| INTS8 | Chromosome 8 open reading frame 52 | 55656 | NM_017864 |
| VPS35 | Vacuolar protein sorting 35 (yeast) | 55737 | NM_018206 |
| HACE1 | Hect domain and ankyrin repeat containing, e3 ubiquitin | 57531 | NM_020771 |
| | protein ligase 1 | | |
| C19orf29 | Chromosome 19 open reading frame 29 | 58509 | XM_944939 |
| SCNM1 | Sodium channel modifier 1 | 79005 | NM_024041 |
| FBXL10 | F-box and leucine-rich repeat protein 10 | 84678 | NM_032590 |
| MGC12966 | Hypothetical protein loc84792 | 84792 | NM_001037163 |
| MGC33212 | Hypothetical protein mgc33212 | 255758 | NM_152773 |
| TIPRLI | Tip41, tor signalling pathway regulator-like (S. cerevisiae) | 261726 | NM_001031800 |
| LOC388610 | Hypothetical loc388610 | 388610 | NM_001013642 |

N.B. Bold = Interactors identified 2x or more

TABLE 4
Correlation of GOLPH3 gene copy number with clinicopathologic and molecular
profiles of NSCLC

| | | | | | | |
|---|---|---|---|---|---|---|
| Correlations | Age | Gender | AC histology | Grade | mTOR | pS6K |

All Histotypes (n = 62)†

| | | | | | | |
|---|---|---|---|---|---|---|
| 5p13 CN† | −0.18 | 0.1 | 0.5 (p < 0.0001) | −0.26 | 0.08 | 0.25 (p = 0.09) |
| mTOR | 0.043 | −0.13 | −0.18 | 0.038 | 1 | 0.42 (p = 0.001) |
| pS6K | −0.09 | −0.17 | −0.27 (p = 0.027) | −0.22 | 0.42 (p = 0.001) | 1 |

AC Histotype (n = 20)†

| | | | | | | |
|---|---|---|---|---|---|---|
| 5p13 CN† | 0.321 | 0.037 | — | −0.286 | 0.343 (p = 0.15) | 0.513 (p = 0.025) |
| mTOR | 0.179 | 0.235 | — | −0.252 | 1 | 0.447 (p = 0.055) |
| pS6K | −0.247 | −0.405 | — | −0.414 | 0.447 (p = 0.055) | 1 |

AC histotype (n = 20)‡

| | | | | | | |
|---|---|---|---|---|---|---|
| 5p13 Status‡ | −0.114 | −0.023 | — | −0.184 | 0.475 (p = 0.04) | 0.724 (p < 0.0001) |

†5p13 CN = copy number (continuous variables) as determined by FISH scores.
‡5p13 Status = Copy number status binarized to Normal (<1.5) or Gain (?1.5) mTOR and pS6K signals based on AQUA scores (see Methods)
Pearson R coefficient is given for parametric correlations between age, mTOR, pS6K protein levels and GOLPH3 gene copy number (continuous variables)
Spearman's rho coefficient is given for non-parametric correlations between continuous variables and GOLPH3 binarized scores, gender, histotype and grade.
Significant correlations with p < 0.05 are in boldface.
Trending p values are indicated in parenthesis.
Not significant p values are not shown.

TABLE 5
AQUA pS6K signals and FISH Ratio on NSCLC TMA.

| | | | | | | pS6K | | |
|---|---|---|---|---|---|---|---|---|
| | | | | | FISH | in | pS6K | pS6K |
| No. | Age | Sex | Organ | Pathology Diagnosis | Ratio | Mask | Nuclear | Cytoplasm |
| 15 | 63 | M | Lung | Adenosquamous carcinoma | 1 | 159.8 | 268.3 | 110.3 |
| 16 | 67 | F | Lung | Adenosquamous carcinoma | 1 | 428.2 | 564.4 | 370.9 |
| 18 | 60 | F | Lung | Adenocarcinoma (grade III) | 1 | 101.9 | 127.5 | 94.2 |
| 20 | 63 | M | Lung | Squamous cell carcinoma (grade II) | 1 | 81.4 | 104.6 | 73.1 |
| 22 | 60 | M | Lung | Adenocarcinoma (grade II) | 7 | 177.1 | 249.1 | 139.3 |
| 23 | 61 | M | Lung | Adenocarcinoma (grade II) | 1 | 108.8 | 214.2 | 79.5 |
| 24 | 54 | F | Lung | Adenocarcinoma (grade III) | 3 | 95.0 | 174.8 | 84.8 |
| 25 | 48 | M | Lung | Gland of tracheal mucous membrane | 1 | 124.0 | 219.1 | 81.1 |
| 26 | 76 | M | Lung | Squamous cell carcinoma (grade II) | 6 | 84.5 | 143.0 | 68.3 |
| 27 | 71 | M | Lung | Squamous cell carcinoma (grade II) | 6 | 177.4 | 291.3 | 152.7 |
| 28 | 46 | F | Lung | Small cell carcinoma | 1 | 280.6 | 529.7 | 173.8 |
| 30 | 54 | M | Lung | Squamous cell carcinoma (grade II) | 1 | 117.8 | 190.1 | 98.8 |
| 31 | 70 | M | Lung | Papillary adenocarcinoma | 6 | 212.1 | 584.6 | 164.9 |
| 32 | 65 | M | Lung | Adenocarcinoma (grade I) | 3 | 173.9 | 267.0 | 137.8 |
| 33 | 48 | M | Lung | Squamous cell carcinoma (grade II) | 1 | 200.6 | 313.1 | 137.4 |
| 34 | 60 | M | Lung | Squamous cell carcinoma (grade II) | 4 | 148.6 | 253.3 | 115.8 |
| 35 | 40 | M | Lung | Squamous cell carcinoma (grade II) | 1 | 266.8 | 233.5 | 291.5 |
| 36 | 67 | M | Lung | Adenocarcinoma (grade II) | 1 | 54.3 | 55.2 | 54.9 |
| 37 | 61 | M | Lung | Tracheal mucous membrane | 1 | 186.3 | 215.1 | 167.6 |
| 38 | 79 | F | Lung | Large cell carcinoma (giant cells) | 1 | 74.7 | 96.0 | 70.3 |
| 39 | 42 | F | Lung | Papillary adenocarcinoma | 2 | 567.7 | 503.5 | 589.5 |
| 41 | 51 | M | Lung | Adenocarcinoma (grade III) | 3 | 126.4 | 219.3 | 105.7 |
| 42 | 62 | M | Lung | Large cell carcinoma | 1 | 194.5 | 335.5 | 155.4 |
| 43 | 59 | M | Lung | Adenocarcinoma (grade II) | 3 | 124.4 | 234.9 | 83.3 |
| 44 | 54 | M | Lung | Squamous cell carcinoma | 7 | 159.3 | 265.7 | 106.8 |
| 45 | 60 | F | Lung | Adenocarcinoma (grade I) | 6 | 533.9 | 335.0 | 582.0 |
| 46 | 65 | M | Lung | Squamous cell carcinoma (grade III) | 1 | 178.3 | 329.9 | 140.4 |
| 47 | 60 | M | Lung | Adenocarcinoma (grade II) | 6 | 214.3 | 293.7 | 189.1 |
| 48 | 54 | M | Lung | Squamous cell carcinoma (grade II) | 2 | 206.0 | 417.6 | 122.5 |
| 49 | 62 | F | Lung | Adenocarcinoma | 2 | 244.0 | 462.2 | 183.1 |
| 50 | 50 | M | Lung | Adenocarcinoma (grade II) | 2 | 636.0 | 527.1 | 689.0 |
| 51 | 55 | M | Lung | Adenocarcinoma (grade II) | 1 | 441.9 | 533.8 | 337.0 |
| 52 | 60 | M | Lung | Squamous cell carcinoma (grade III) | 2 | 155.6 | 295.4 | 96.9 |
| 53 | 44 | M | Lung | Squamous cell carcinoma (grade II) | 7 | 114.0 | 168.2 | 94.6 |
| 55 | 65 | M | Lung | Bronchioloalveolar carcinoma | 1 | 106.3 | 186.8 | 72.0 |
| 56 | 24 | M | Lung | Squamous cell carcinoma (grade II) | 2 | 144.1 | 223.9 | 118.4 |
| 58 | 54 | F | Lung | Squamous cell carcinoma (grade III) | 1 | 250.2 | 351.6 | 200.7 |
| 59 | 25 | M | Lung | Small cell carcinoma | 1 | 145.1 | 239.9 | 95.3 |
| 60 | 66 | F | Lung | Adenocarcinoma (grade II) | 8 | 524.8 | 359.3 | 625.1 |
| 61 | 59 | M | Lung | Adenocarcinoma (grade II) | 6 | 128.6 | 178.5 | 116.8 |
| 62 | 47 | M | Lung | Squamous cell carcinoma (grade II) | 2 | 178.5 | 286.3 | 139.8 |
| 64 | 68 | M | Lung | Squamous cell carcinoma (grade II) | 1 | 197.0 | 266.4 | 195.8 |
| 66 | 50 | M | Lung | Squamous cell carcinoma (grade II) | 2 | 98.6 | 159.6 | 98.2 |
| 67 | 63 | M | Lung | Small cell carcinoma | 1 | 121.2 | 149.7 | 114.0 |
| 68 | 60 | M | Lung | Squamous cell carcinoma (grade II) | 1.5 | 300.9 | 378.5 | 285.6 |
| 69 | 57 | M | Lung | Squamous cell carcinoma (grade II) | 2 | 187.9 | 242.5 | 168.9 |

### Example 7

### GOLPH3 Activates mTOR Signaling

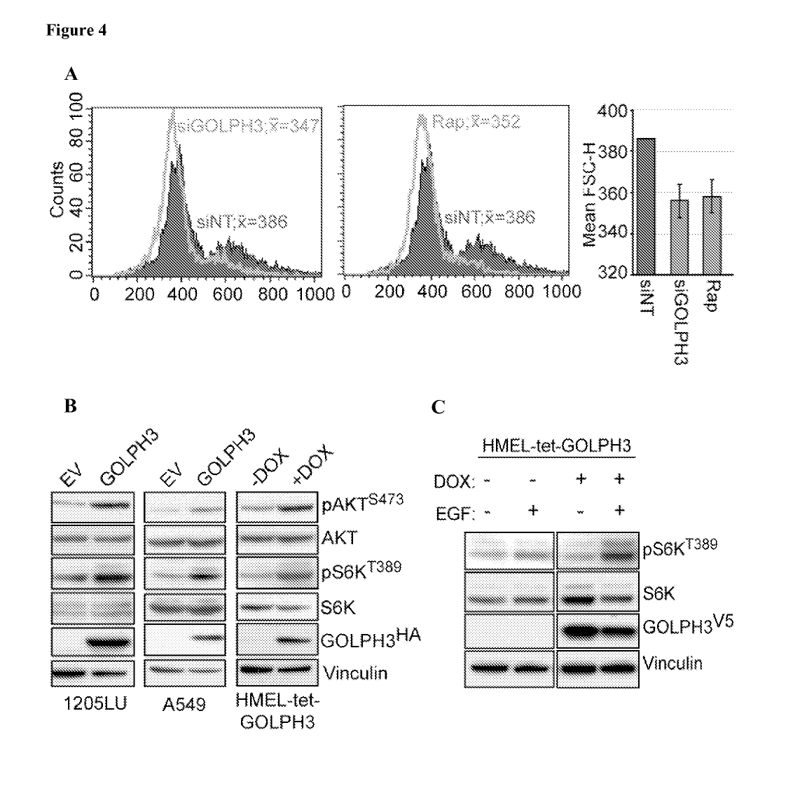

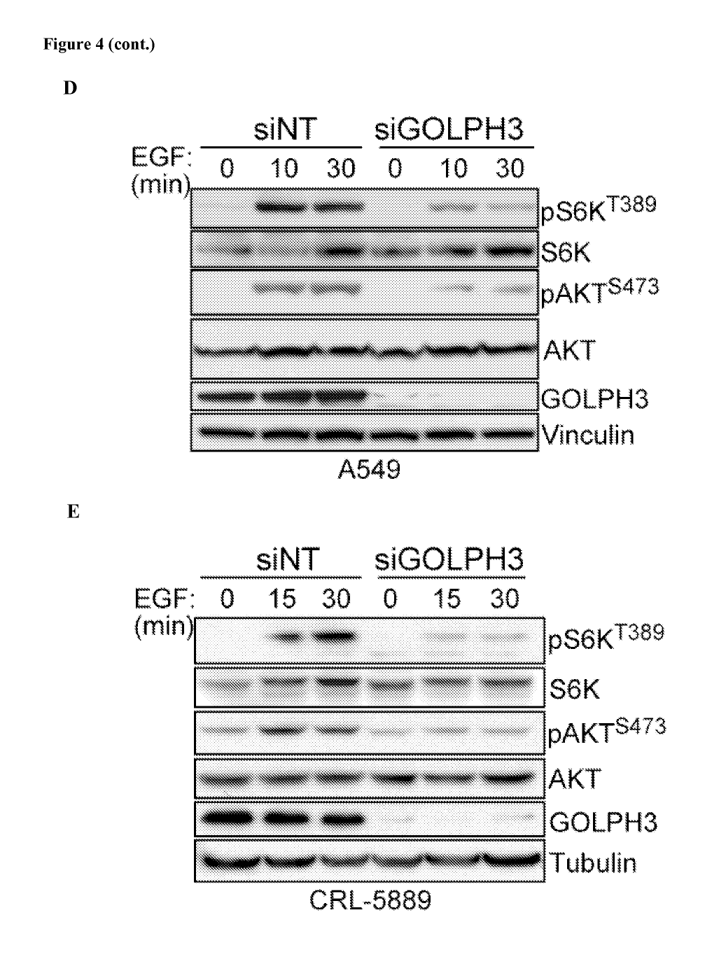

To test the hypothesis that GOLPH3 activates mTOR signaling, the biological consequences of GOLPH3 modulation was first examined. Consistent with mTOR's role in cell size regulation, RNAi-mediated GOLPH3 depletion led to a significant cell size reduction in A549, an effect that was comparable to treatment with rapamycin (Fingar et al. (2002) Genes Dev 16: 1472-1487; FIG. 4A). Next, the biochemical consequences of GOLPH3 modulation was assayed.

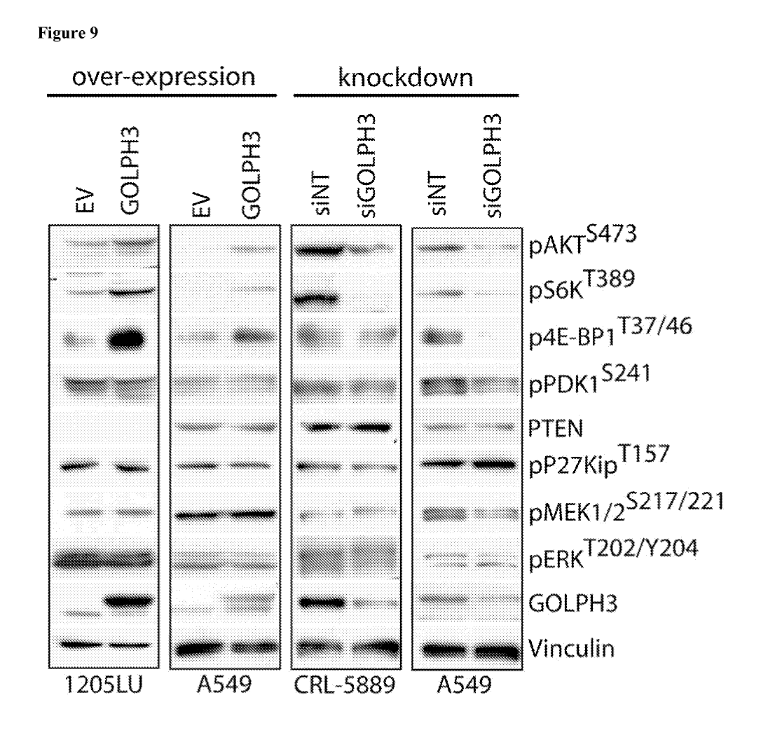

Since the mTOR substrate S6K is a kinase effector of cell size that is phosphorylated at Thr389 by mTOR (Burnett et al. (1998) Proc. Natl. Acad. Sci. USA 95: 1432-1437; Isotani et al. (1999) J. Biol. Chem. 274: 34493-34498), phospho-S6K status was investigated as a readout of the mTORC1 axis. Consistent with the human tumor data showing elevated pS6K in 5p13 amplified NSCLC specimens, GOLPH3 over-expression resulted in elevated pS6K in tumor cell lines (1205LU and A549) as well as in HMEL-tet-GOLPH3, a TERT-immortalized human melanocyte cell line engineered with a tet-regulated GOLPH3 expression construct (FIG. 4B). Substantiating these observations using the inducible system, GOLPH3 induction further enhanced pS6K accumulation in response to growth factor stimulation by epidermal growth factor (EGF; FIG. 4C). At the same time, monitored phosphorylation of AKT (pAKT) was monitored at Ser473, a direct substrate of mTORC2 (Hresko et al. (2005) J. Biol. Chem. 280: 40406-40416; Sarbassov et al. (2005) Science 307: 1098-1101). Similar to mTORC1-mediated phosphorylation of S6K, a comparable increase in pAKT phosphorylation was observed in GOLPH3 over-expressing cells (FIG. 4B), suggesting that GOLPH3 can enhance signaling through both mTOR-associated complexes. Moreover, AKT and S6K phosphorylation was significantly abrogated in siGOLPH3-treated NSCLC A549 and CRL-5889 cells compared to control cells in response to EGF (FIGS. 4D-4E). Additional biochemical analyses showed altered phosphorylation of the mTOR substrates S6K^{Thr389}, p4E-BP1^{Thr37/46} and AKT^{Ser473} with little to no affect on other signaling proteins including PTEN, MEK1/2 and p44/42 (Erk1/2) among others (FIG. 9). Collectively, these data biochemically indicate that GOLPH3 activates mTOR signaling through phosphorylation of both mTORC1- and mTORC2-specific substrates.

### Example 8

### GOLPH3 Modulates Rapamycin Sensitivity

Complementing the described genetic studies, it was next asked whether GOLPH3 expression levels affected tumor cell sensitivity to pharmacological mTOR inhibition in vivo. Here, two human melanoma cells, 1205LU and WM239A, were selected based on their normal GOLPH3 copy-number and low protein expression as well as their ability to readily form subcutaneous (SQ) tumors in vivo. Parental cells were engineered to stably express either empty vector (EV) or GOLPH3 for orthotopic subcutaneous transplantation into immunodeficient animals for tumor growth. Next, the degree of tumor growth inhibition (% TGI) was compared in GOLPH3-expressing versus EV-control tumors upon rapamycin treatment.

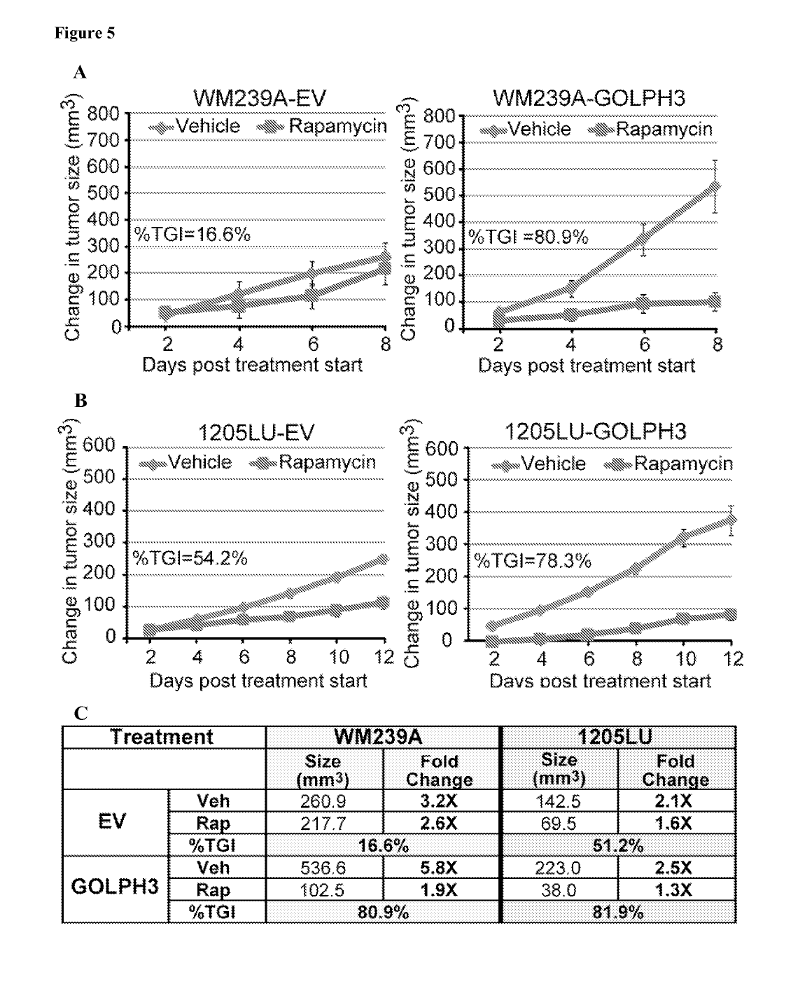

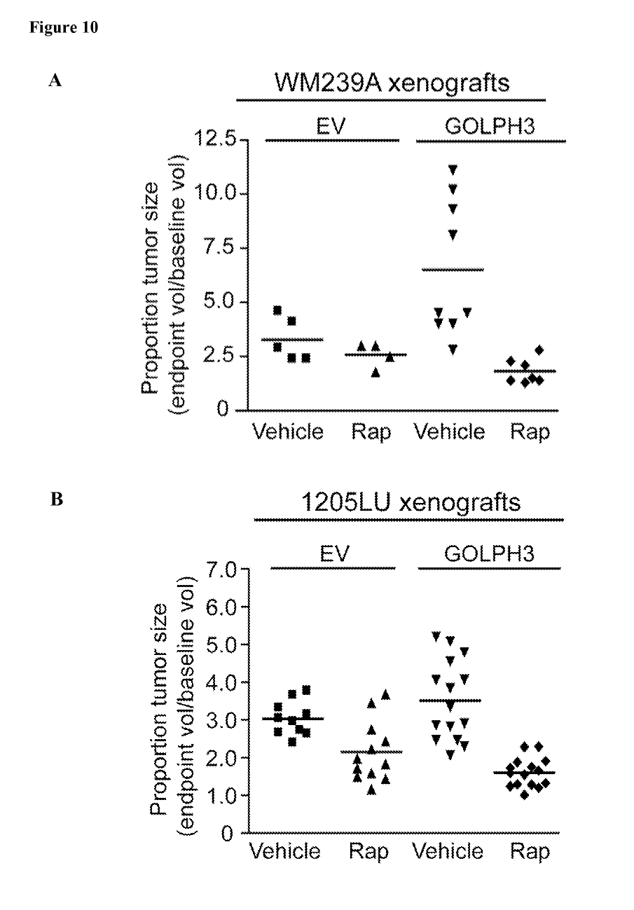

Consistent with above (FIG. 2E), 1205LU-GOLPH3 cells exhibited a significant growth advantage compared to 1205LU-EV control cells in vivo (1.9-fold increase in tumor volume at 36 days post-injection in vehicle control cohort, p-value=0.0148). Upon tumors reaching a baseline volume of ˜100 mm³, the animals were randomized into control and treatment cohorts for intraperitoneal injection of either vehicle or rapamycin (6.0 mg/kg) every other day. The treatment trial was terminated when one animal in any cohort had to be sacrificed for tumor burden according to IACUC regulations. The inhibitory effect of rapamycin on mTOR activity of treated tumors was verified by Western analysis (FIG. 7G). The efficacy of rapamycin treatment was then calculated as % TGI of treated versus non-treated cohorts after 4 doses (day 8 of trial) for WM239A and 6 doses (day 12 of trial) for 1205LU. Indeed, GOLPH3-expressing tumors were significantly more sensitive to rapamycin in vivo (FIGS. 5A-5C and FIGS. 10A-10B). Therefore, GOLPH3's biochemical effect on mTOR signaling is a critical aspect of its oncogenic function, as inhibition by rapamycin effectively blocked the growth advantage conferred by GOLPH3 in vivo.

Thus, integrative analyses of genome-wide copy number and expression data coupled with reinforcing knockdown and over-expression assays in vitro and in vivo led to the identification of GOLPH3 as a bona fide oncoprotein frequently targeted for copy number gain/amplification in diverse human cancers. A suspect role for the Golgi apparatus in regulating cancer-relevant signaling has been speculated based on observation that some cytoplasmic membrane oncoproteins, such as RAS, can functionally signal when temporally present at the Golgi apparatus (Chiu et al. (2002) Nat. Cell. Biol. 4: 343-350). However, proteins such as GOLPH3 that are predominantly localized to the TGN have not been directly linked on a genetic level to cancer; therefore, GOLPH3 represents a first-in-class Golgi oncoprotein. Mechanistically, enhanced activation of mTOR signaling represents a molecular basis for GOLPH3's oncogenic activity. In this light, enhanced and sustained mTOR activation in vivo would be expected to confer a significant growth advantage to cancer cells, a likely basis for increased GOLPH3 gene copy number or expression in a large fraction of human cancers.

The molecular data on the physical interaction between GOLPH3 and the retromer complex, which is responsible for protein trafficking between endosomes and the TGN (Bonifacino et al. (2008) Curr. Opin. Cell. Biol. 20: 427-436), for the first time genetically implicates this biological process in cancer. This is consistent with recent reports on the essential role of the retromer and retrograde transport in regulation of the Wntless receptor and proper secretion of the WNT morphogen (Eaton (2008) Dev. Cell 14: 4-6), which is important in both normal and neoplastic development. Along the same line, depletion of VPS35 in Drosophila inhibited endocytosis of RTKs with concomitant alterations in downstream signaling (Korolchuk et al. (2007) J. Cell Sci. 120: 4367-4376). Taken together, GOLPH3 might function with VPS35 and the retromer to regulate receptor recycling of key molecules thereby influencing downstream signaling through mTOR.

It has recently been discovered that Vps74, the yeast homolog of GOLPH3, is required for proper docking and localization of glycosyltransferases to the Golgi apparatus (Schmitz et al. (2008) Dev. Cell 14: 523-534; Tu et al. (2008) Science 321: 404-407). Protein glycosylation is one of the most prevalent forms of post-translational modification, and altered glycosylation is a hallmark feature of cancers (Ohtsubo et al. (2006) Cell 126: 855-867). It is noteworthy that glycosylation is known to be important for growth factor-activation of transmembrane receptors, since glycosylation mediates receptor sorting, ligand binding and endocytosis (Ohtsubo et al. (2006) Cell 126: 855-867; Takahashi et al. (2004) Glycoconj. J. 20: 207-212). Thus, it is plausible that human GOLPH3 might serve a similar function in glycosyltransferase docking as in S. cerevisiae and therefore might influence the downstream mTOR signaling response through its effect on membrane RTKs.

The PI3K-AKT-mTOR signaling cascade is activated in nearly all cancers and hence represents an intense focus for cancer drug development. However, the clinical response to rapamycin and its analogs has been feeble (Sabatini et al. Nat Rev Cancer 6 (9), 729-734 (2006). GOLPH3's role in activating mTOR signaling and conferring increased sensitivity to rapamycin in preclinical setting, as described herein, indicates that GOLPH3 expression level or copy number status may predict sensitivity to mTOR inhibitors. Indeed, endpoint analysis of the described preclinical treatment studies showed that rapamycin was significantly more effective against xenograft tumors expressing high level of GOLPH3 (p=0.0268; 1205LU-GOLPH3 vs. 1205LU-EV tumor volumes at endpoint; FIG. 10B), thereby indicating that GOLPH3 levels may be a positive predictor of rapamycin sensitivity.

### Example 9

### GOLPH3 Depletion Reduces Lipid Second Messenger Production and Modulates the PI3K Pathway

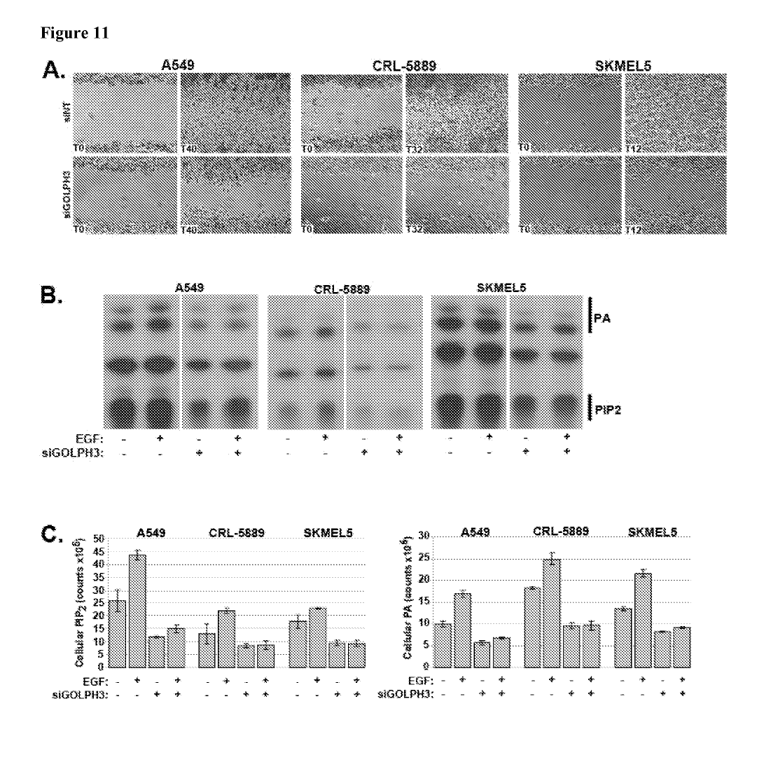

Depletion of GOLPH3 was shown to reduce cell migration, possibly through either an mTOR- or phospholipid-mediated pathway (FIG. 11A). GOLPH3 depletion was also shown to reduce in vivo basal and growth factor stimulated biosynthesis of lipid second messengers that feed into cancer signaling pathways (FIGS. 11B-11C).

### Example 10

### Growth Factor Signaling Causes GOLPH3 Mis-Localization via ARF4

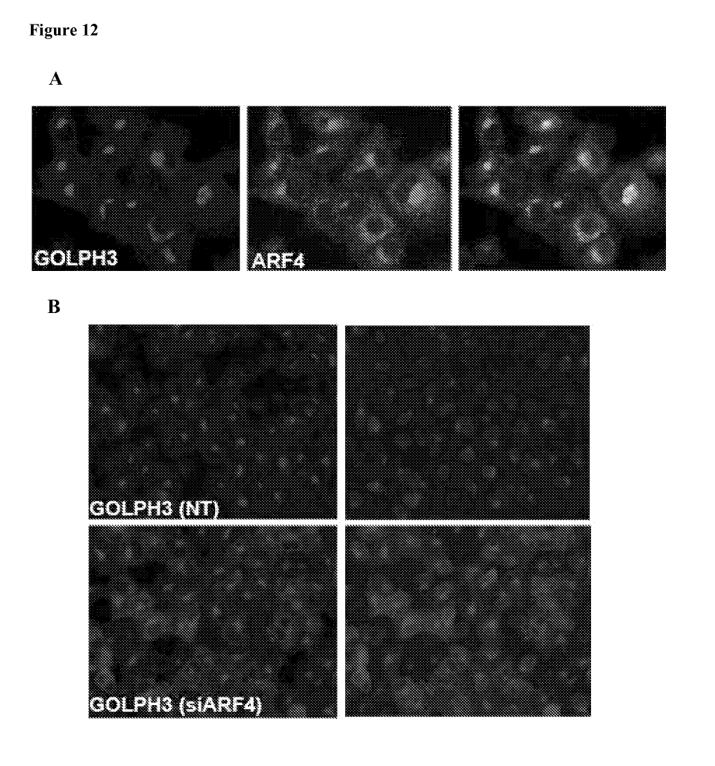

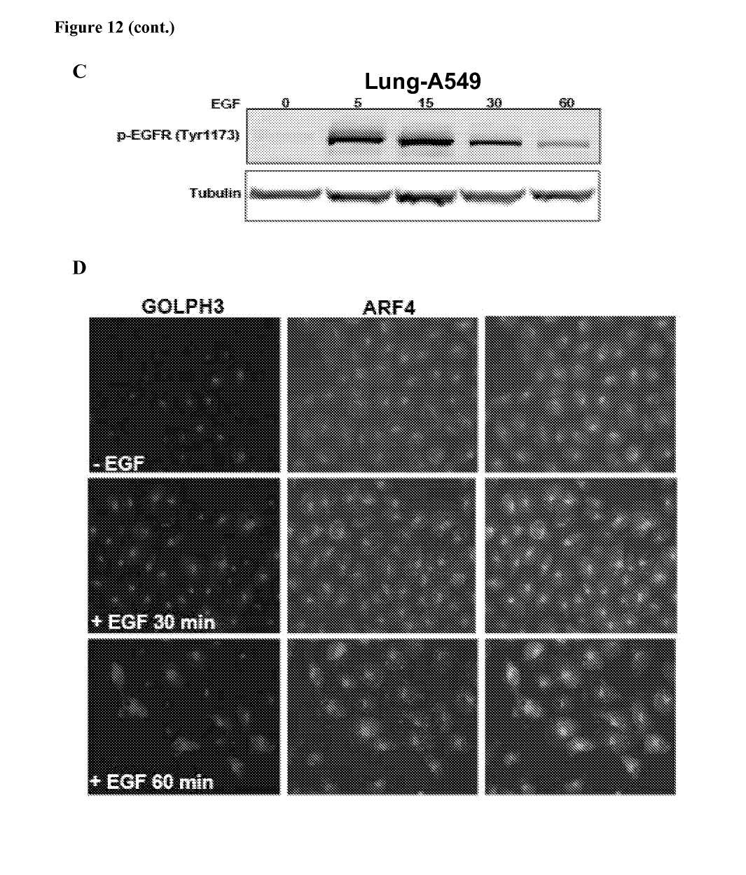

Using immunofluorescence assays in lung A549 cells, ARF4 was shown to co-localize with GOLPH3 (FIG. 12A). In order to determine whether or not ARF4 might regulate GOLPH3 localization at the Golgi apparatus, ARF4 siRNA were transfected into A549 cells. GOLPH3 was shown to leave the Golgi with ARF4 siRNA, demonstrating that ARF4 is the GTPase required for GOLPH3 phosphorylation (FIG. 12B). Lastly, to demonstrate whether GOLPH3 localization is altered upon EGFR stimulation, A549 cells were treated with EGF at specific time points and GOLPH3 localization was monitored via immunofluorescence. It was shown that EGF caused redistribution of GOLPH3 from the Golgi (FIGS. 12C-12D). Taken together, these results demonstrate that growth factors stimulate ARF4 movement to the membranes (Kim et al. (2003) J. Biol. Chem. 278:2661-2668), thus, regulating GOLPH3 redistribution from the Golgi.

Without being bound by theory, one mechanism by which GOLPH3 likely regulates signaling relates to its association with VPS35, a central component of the retromer complex that plays a key role in recycling of transmembrane receptors. The retromer complex is important in cancer as in, for example, the ability of the retromer complex to regulate secretion of Wnt family proteins, which provide important roles in developmental processes and cancer pathogenesis ((Belenkaya et al. (2008) Dev. Cell 14: 120-131; Franch-Marro et al. (2008) Nat. Cell Biol. 10:170-177; Pan et al. (2008) Dev. Cell 14:132-139; Port et al. (2008) Nat. Cell Biol. 10: 178-185; Yang et al. (2008) Dev. Cell 14:140-147); Clevers (2006) Cell 3:469-480) Inhibiting retromer function through depletion of VPS35 destabilizes Wntless, the transmembrane protein that regulates Wnt secretion, by impeding its recycling and further use following internalization. In another example, a genetic screen in Drosophila discovered a role for VPS35 in regulating Racl-dependent actin polymerization (Korolchuk et al. (2007) J. Cell Sci. 120:4367-4376). Depletion of VPS35 was found to inhibit endocytosis of several transmembrane proteins that include the Toll receptor, EGFR and the PDGF and VEGF-receptor-related receptor (PVR) with concomitant increase in plasma membrane localization and correspondingly increase in signaling via downstream components. These combined data indicate that GOPLH3 functions with VPS35 to regulate receptor recycling of key molecules thus influencing downstream signaling through AKT/mTOR.

Based upon the examples described above (e.g., through the yeast two-hybrid screen), it was found that GOLPH3 interacts with the ARF4 GTPase that regulates retrograde protein transport of cell surface receptors and other proteins. ARF4 cycles from the Golgi apparatus to the plasma membrane where it binds receptor tyrosine kinases upstream of Akt/mTOR (e.g., EGFR) (Kim et al. (2003) J. Biol. Chem. 278:2661-2668). In addition, the examples described above demonstrate that 1) ARF4 and GOLPH3 colocalize at the Golgi, 2) depletion of ARF4 with RNAi causes GOLPH3 to relocalize outside the Golgi (indicating that the GTPase activity is required for GOLPH3 localization, which is phosphorylation-dependent, at the Golgi), and 3) GOLPH3 localization is altered upon EGFR stimulation with EGF. These data provide a model whereby EGFR stimulation causes ARF4 redistribution from the Golgi to the plasma membrane (Kim et al. (2003) J. Biol. Chem. 278:2661-2668), and the resulting loss of ARF4 GTPase activity at the Golgi phenocopies ARF4 KD resulting in GOLPH3 redistribution to the cytoplasm. Taken together with the fact that GOLPH3 has been shown to dynamically associate with the Golgi, moving to and from the cytoplasm in a GOLPH3 phosphorylation-dependent manner (Snyder et al. (2006) Mol. Biol. Cell 17:511-524), these results indicate that GOLPH3, ARF4 and the retromer complex cooperated to regulate receptor recycling in response to growth factor stimulation.

Without being bound by theory, GOLPH3 also likely regulates signaling similar to VPS74, the yeast homolog of which regulates glycosyltransferase localization to the Golgi apparatus (Schmitz et al. (2008) Dev. Cell 14:523-534; Tu et al. (2008) Science 321:404-407). Protein glycosylation is one of the most prevalent forms of post-translational modification, and altered glycosylation is a hallmark feature of tumorigenesis (Ohtsubo et al. (2006) Cell 126:855-867). Glycan structures are well-known markers for tumor progression and are associated with numerous pathological events in cancer that include cell growth, adhesion, migration and invasion, immune recognition and signal transduction. In the case of signal transduction, glycosylation has been proven important for growth factor-activation of transmembrane receptors (Takahashi et al. (2004) Glycoconj. J. 20:207-212). For example, EGFR contains 12 N-glycosylation consensus sites (Carpenter and Cohen (1990) J. Biol. Chem. 265:7709-7712), and glycosylation at these residues is necessary for both EGFR sorting and subsequent ligand binding (Soderquist and Carpenter (1984) J. Biol. Chem. 259:12586-12594; Gamou and Shimizu (1988) J. Biochem. 104:388-396). Moreover, Golgi glycosyltransferase activity can alter endocytosis of transmembrane receptors, which can lead to altered sensitivity to receptor ligand (Ohtsubo et al. (2006) Cell 126:855-867). In the case of EGFR, modified N-glycosylation sequesters EGFR at the plasma membrane by resisting internalization thereby resulting in prolonged responsiveness to growth factor (Partidge et al. (2004) Science 306:120-124).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of assessing whether a subject is afflicted with cancer or is at risk for developing cancer, the method comprising comparing the copy number of a marker in a subject sample to the normal copy number of the marker, wherein said marker comprises region 5p13 of human chromosome 5 or a fragment thereof, and wherein an altered copy number of the marker in the sample indicates that the subject is afflicted with cancer or at risk for developing cancer.

2. The method of claim 1, wherein the copy number is assessed by fluorescent in situ hybridization (FISH), quantitative PCR (qPCR), or single-molecule sequencing.

3. (canceled)

4. The method of claim 1, wherein the normal copy number is obtained from a control sample.

5. A method of assessing whether a subject is afflicted with cancer or is at risk for developing cancer, the method comprising comparing:
a) the amount, structure, subcellular localization, and/or activity of a marker in a subject sample, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and
b) the normal amount, structure, subcellular localization, and/or activity of the marker,
wherein a significant difference in the amount, structure, subcellular localization, and/or activity of the marker in the sample and the normal amount, structure, subcellular localization, and/or activity is an indication that the subject is afflicted with cancer or at risk for developing cancer.

6. The method of claim 5, wherein the marker is GOLPH3.

7. The method of claim 6, wherein GOLPH3 increases cellular phospholipids, modulates retrograde trafficking by the retromer, modulates the PI3K pathway, or modulates receptor recycling.

8. The method of claim 7, wherein the cellular phospholipids are selected from the group consisting of PIP₂ and PA.

9. (canceled)

10. The method of claim 6, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

11-12. (canceled)

13. The method of claim 5, wherein the amount, structure, subcellular localization, or activity of a marker is compared.

14-16. (canceled)

17. The method of claim 13, wherein the amount of the marker is determined by determining the level of expression of the marker, determining germline copy number of the marker, or determining somatic copy number of the marker.

18. (canceled)

19. The method of claim 5, wherein the normal amount, subcellular localization, structure, and/or activity is obtained from a control sample.

20. The method of claim 1 or 5, wherein the sample is selected from the group consisting of tissue, whole blood, serum, plasma, buccal scrape, saliva, cerebrospinal fluid, urine, stool, and bone marrow.

21. The method of claim 17, wherein the copy number is assessed by fluorescence in situ hybridization (FISH), quantitative PCR (qPCR), comparative genomic hybridization (CGH), or single-molecule sequencing.

22-23. (canceled)

24. The method of claim 17, wherein the level of expression of the marker in the sample is assessed by detecting the presence in the sample of a protein corresponding to the marker.

25. The method of claim 24, wherein the presence of the protein is detected using a reagent which specifically binds with the protein, optionally wherein the reagent is selected from the group consisting of an antibody, an antibody derivative, and an antibody fragment.

26. (canceled)

27. The method of claim 17, wherein the level of expression of the marker in the sample is assessed by detecting the presence in the sample of a transcribed polynucleotide or portion thereof, wherein the transcribed polynucleotide comprises the marker.

28. The method of claim 27, wherein the transcribed polynucleotide is an mRNA or cDNA or wherein the step of detecting further comprises amplifying the transcribed polynucleotide.

29-30. (canceled)

31. The method of claim 17, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

32. A method of assessing the likelihood of efficacy of an mTOR pathway inhibitor in a subject, the method comprising comparing:
a) the amount, structure, subcellular localization, and/or activity of a marker in a subject sample, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and
b) the normal amount, structure, subcellular localization, and/or activity of the marker,
wherein a significant difference in the amount, structure, subcellular localization, and/or activity of the marker in the sample and the normal amount, structure, subcellular localization, and/or activity is an indication that an mTOR pathway inhibitor is likely to have significant efficacy in the subject.

33. The method of claim 32, wherein the marker is GOLPH3.

34. The method of claim 33, wherein GOLPH3 increases cellular phospholipids, modulates retrograde trafficking by the retromer, modulates the PI3K pathway, or modulates receptor recycling.

35. The method of claim 34, wherein the cellular phospholipids are selected from the group consisting of PIP₂ and PA.

36. (canceled)

37. The method of claim 33, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

38. (canceled)

39. The method of claim 33, wherein GOLPH3 translocates from the Golgi to the plasma membrane after exposure of the subject sample to EGF.

40. The method of claim 32, wherein the mTOR pathway inhibitor is rapamycin.

41. The method of claim 32, wherein the amount, structure, subcellular localization, or activity of a marker is compared.

42-44. (canceled)

45. The method of claim 41, wherein the amount of the marker is determined by determining the level of expression of the marker, determining germline copy number of the marker, or determining somatic copy number of the marker.

46. (canceled)

47. The method of claim 32, wherein the normal amount, subcellular localization, structure, and/or activity is obtained from a control sample.

48. The method of claim 32, wherein the sample is selected from the group consisting of tissue, whole blood, serum, plasma, buccal scrape, saliva, cerebrospinal fluid, urine, stool, and bone marrow.

49. The method of claim 45, wherein the copy number is assessed by fluorescence in situ hybridization (FISH), quantitative PCR (qPCR), comparative genomic hybridization (CGH), or single-molecule sequencing.

50-51. (canceled)

52. The method of claim 45, wherein the level of expression of the marker in the sample is assessed by detecting the presence in the sample of a protein corresponding to the marker.

53. The method of claim 52, wherein the presence of the protein is detected using a reagent which specifically binds with the protein, optionally wherein the reagent is selected from the group consisting of an antibody, an antibody derivative, and an antibody fragment.

54. (canceled)

55. The method of claim 45, wherein the level of expression of the marker in the sample is assessed by detecting the presence in the sample of a transcribed polynucleotide or portion thereof, wherein the transcribed polynucleotide comprises the marker.

56. The method of claim 55, wherein the transcribed polynucleotide is an mRNA or cDNA or wherein the step of detecting further comprises amplifying the transcribed polynucleotide.

57-58. (canceled)

59. The method of claim 45, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

60. A method for monitoring the progression of cancer in a subject, the method comprising:
a) detecting in a subject sample at a first point in time, the amount, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3;
b) repeating step a) at a subsequent point in time; and
c) comparing the amount, subcellular localization, and/or activity detected in steps a) and b), and thereby monitoring the progression of cancer in the subject.

61. The method of claim 60, wherein the marker is GOLPH3.

62. The method of claim 61, wherein GOLPH3 increases cellular phospholipids, modulates retrograde trafficking by the retromer, modulates the PI3K pathway, or modulates receptor recycling.

63. The method of claim 62, wherein the cellular phospholipids are selected from the group consisting of PIP₂ and PA.

64. (canceled)

65. The method of claim 61, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

66-67. (canceled)

68. The method of claim 60, wherein the sample is selected from the group consisting of tissue, whole blood, serum, plasma, buccal scrape, saliva, cerebrospinal fluid, urine, stool, and bone marrow.

69. The method of claim 60, wherein the activity, subcellular localization, or amount of a marker is determined.

70-71. (canceled)

72. The method of claim 69, wherein the amount of the marker is determined by determining the level of expression of the marker.

73. The method of claim 69, wherein the level of expression of the marker in the sample is assessed by detecting the presence in the sample of a protein corresponding to the marker.

74. The method of claim 73, wherein the presence of the protein is detected using a reagent which specifically binds with the protein, optionally wherein the reagent is selected from the group consisting of an antibody, an antibody derivative, and an antibody fragment.

75. (canceled)

76. The method of claim 72, wherein the level of expression of the marker in the sample is assessed by detecting the presence in the sample of a transcribed polynucleotide or portion thereof, wherein the transcribed polynucleotide comprises the marker.

77. The method of claim 76, wherein the transcribed polynucleotide is an mRNA or cDNA or wherein the step of detecting further comprises amplifying the transcribed polynucleotide.

78-79. (canceled)

80. The method of claim 72, wherein the level of expression of the marker in the sample is assessed by detecting the presence in the sample of a transcribed polynucleotide which anneals with the marker or anneals with a portion of a polynucleotide wherein the polynucleotide comprises the marker, under stringent hybridization conditions.

81. The method of claim 60, wherein the sample comprises cells obtained from the subject.

82. The method of claim 60, wherein between the first point in time and the subsequent point in time, the subject has undergone treatment for cancer, has completed treatment for cancer, and/or is in remission.

83. A method of assessing the efficacy of a test compound for inhibiting cancer in a subject, the method comprising comparing:
a) the amount, subcellular localization, and/or activity of a marker in a first sample obtained from the subject and maintained in the presence of the test compound, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and
b) the amount, subcellular localization, and/or activity of the marker in a second sample obtained from the subject and maintained in the absence of the test compound,
wherein a significant difference in the amount, subcellular localization, and/or activity of a marker in the first sample relative to the second sample, is an indication that the test compound is efficacious for inhibiting cancer in the subject.

84. The method of claim 83, wherein the marker is GOLPH3.

85. The method of claim 84, wherein GOLPH3 increases cellular phospholipids, modulates retrograde trafficking by the retromer, modulates the PI3K pathway, or modulates receptor recycling.

86. The method of claim 85, wherein the cellular phospholipids are selected from the group consisting of PIP₂ and PA.

87. (canceled)

88. The method of claim 84, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

89-90. (canceled)

91. The method of claim 83, wherein the first and second samples are portions of a single sample or portions of pooled samples obtained from the subject.

92. (canceled)

93. A method of assessing the efficacy of a therapy for inhibiting cancer in a subject, the method comprising comparing:
a) the amount, subcellular localization, and/or activity of a marker in a first sample obtained from the subject prior to providing at least a portion of the therapy to the subject, wherein the marker is selected from the group consisting of markers which reside within region 5p13 of human chromosome 5, markers which reside within the MCR consisting of 32.0 Mb to 32.8 Mb of human chromosome 5, and markers listed in Table 3, and
b) the amount, subcellular localization, and/or activity of the marker in a second sample obtained from the subject following provision of the portion of the therapy,
wherein a significant difference in the amount, subcellular localization, and/or activity of a marker in the first sample relative to the second sample, is an indication that the therapy is efficacious for inhibiting cancer in the subject.

94. The method of claim 93, wherein the marker is GOLPH3.

95. The method of claim 94, wherein GOLPH3 increases cellular phospholipids, modulates retrograde trafficking by the retromer, modulates the PI3K pathway, or modulates receptor recycling.

96. The method of claim 95, wherein the cellular phospholipids are selected from the group consisting of PIP₂ and PA.

97. (canceled)

98. The method of claim 94, wherein GOLPH3 is phosphorylated by ARF4.

99. The method of claim 94, wherein GOLPH3 phosphorylation levels are reduced after exposure of the subject sample to EGF.

100. The method of claim 94, wherein GOLPH3 translocates from the Golgi to the plasma membrane after exposure of the subject sample to EGF.

101. A method of selecting a composition capable of modulating cancer, the method comprising:
a) obtaining a sample comprising cancer cells;
b) contacting said cells with a test compound; and
c) determining the ability of the test compound to modulate the amount, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3,
thereby identifying a modulator of cancer.

102. The method of claim 101, wherein the marker is GOLPH3.

103. The method of claim 102, wherein GOLPH3 increases cellular phospholipids, modulates retrograde trafficking by the retromer, modulates the PI3K pathway, or modulates receptor recycling.

104. The method of claim 103, wherein the cellular phospholipids are selected from the group consisting of PIP₂ and PA.

105. (canceled)

106. The method of claim 102, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

107-108. (canceled)

109. The method of claim 101, wherein said cells are isolated from an animal model of cancer, a cancer cell line, or a subject suffering from cancer.

110-111. (canceled)

112. The method of claim 109, wherein said cells are from cell lines selected from the group consisting of lung carcinoma, ovarian carcinoma, melanoma, breast carcinoma, colon carcinoma, multiple myeloma, prostate carcinoma, pancreatic carcinoma, and liver carcinoma cell lines.

113. A method of selecting a composition capable of modulating cancer, the method comprising:
a) contacting a marker with a test compound, wherein the marker is selected from the group consisting of markers which reside within region 5p13 of human chromosome 5, markers which reside within the MCR consisting of 32.0 Mb to 32.8 Mb of human chromosome 5, and markers listed in Table 3; and
b) determining the ability of the test compound to modulate the amount, subcellular localization, and/or activity of a marker which resides in the MCR,
thereby identifying a composition capable of modulating cancer.

114. The method of claim 113, wherein the marker is GOLPH3.

115. The method of claim 114, wherein GOLPH3 increases cellular phospholipids, modulates retrograde trafficking by the retromer, modulates the PI3K pathway, or modulates receptor recycling.

116. The method of claim 115, wherein the cellular phospholipids are selected from the group consisting of PIP₂ and PA.

117. (canceled)

118. The method of claim 114, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

119-120. (canceled)

121. The method of claim 101 or 113, further comprising administering the test compound to an animal model of cancer or wherein the modulator changes the subcellular localization or inhibits the amount and/or activity of a gene or protein corresponding to GOLPH3.

122. (canceled)

123. A method of treating a subject afflicted with cancer comprising administering to the subject a compound which changes the subcellular localization of or modulates the amount and/or activity of a gene or protein corresponding to a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3.

124. The method of claim 123, wherein the marker is GOLPH3.

125. The method of claim 124, wherein GOLPH3 increases cellular phospholipids, modulates retrograde trafficking by the retromer, modulates the PI3K pathway, or modulates receptor recycling.

126. The method of claim 125, wherein the cellular phospholipids are selected from the group consisting of PIP₂ and PA.

127. (canceled)

128. The method of claim 124, wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

129-130. (canceled)

131. The method of claim 123, wherein said compound is administered in a pharmaceutically acceptable formulation.

132. The method of claim 123, wherein said compound is an antibody or an antigen binding fragment thereof, which specifically binds to a protein corresponding to said marker, optionally wherein said antibody is conjugated to a toxin or wherein said antibody is conjugated to a chemotherapeutic agent.

133-134. (canceled)

135. The method of claim 123, wherein said compound is an RNA interfering agent which inhibits expression of a gene corresponding to said marker, optionally wherein said RNA interfering agent is an siRNA molecule or an shRNA molecule.

136. (canceled)

137. The method of claim 123, wherein said compound is selected from the group consisting of an antisense oligonucleotide complementary to a gene corresponding to said marker, a peptide or peptidomimetic, a small molecule which inhibits activity of said marker, and an apatmer which inhibits expression or activity of said marker.

138-142. (canceled)

143. A kit for assessing the ability of a compound to inhibit cancer or for assessing whether a subject is afflicted with cancer, the kit comprising a reagent for assessing the amount, structure, subcellular localization, and/or activity of a marker, wherein the marker is selected from the group consisting of markers which reside within region 5p13 of human chromosome 5, markers which reside within the MCR consisting of 32.0 Mb to 32.8 Mb of human chromosome 5, and markers listed in Table 3, optionally wherein the kit comprises (a) a reagent for assessing the copy number of a marker, wherein the marker comprises region 5p13 of human chromosome 5 or a fragment thereof, (b) an antibody or an fragment thereof that specifically binds with a protein corresponding to the marker, or (c) a nucleic acid probe wherein the probe specifically binds with a transcribed polynucleotide corresponding to the marker.

144-146. (canceled)

147. The kit of any one claim 143, wherein the marker is GOLPH3.

148. The kit of claim 143, wherein the marker is GOLPH3 and wherein GOLPH3 increases cellular phospholipids, modulates retrograde trafficking by the retromer, modulates the PI3K pathway, or modulates receptor recycling.

149. The kit of claim 148, wherein the cellular phospholipids are selected from the group consisting of PIP₂ and PA.

150. (canceled)

151. The kit of claim 143, wherein the marker is GOLPH3 and wherein GOLPH3 is phosphorylated by ARF4, GOLPH3 phosphorylation levels are reduced after exposure of the subject sample to EGF, or GOLPH3 translocates from the Golgi to the plasma membrane after exposure of the subject sample to EGF.

152-153. (canceled)
